(19) United States
(12) Patent Application Publication
(10) Pub. No.: US 2024/0352428 A1
(43) Pub. Date: Oct. 24, 2024

(54) BACTERIOPHAGES WITH IMPROVED ANTIMICROBIAL ACTIVITY

(71) Applicant: ARMATA PHARMACEUTICALS, INC., Marina del Rey, CA (US)

(72) Inventors: Sébastien LEMIRE, Culver City, CA (US); Angela B. SORIAGA, Culver City, CA (US); Katrina Tram Anh NGUYEN, Playa Vista, CA (US)

(21) Appl. No.: 18/294,138

(22) PCT Filed: Aug. 2, 2022
(86) PCT No.: PCT/US22/74446

§ 371 Date: Jan. 31, 2024

Related U.S. Application Data
(60) Provisional application No. 63/228,504, filed on Aug. 2, 2021.

(51) Int. Cl. C12N 7/00; C12N 9/22; C12N 9/88; C12N 15/11; C12N 15/90

(52) U.S. Cl. CPC C12N 7/00; C12N 9/22; C12N 9/88; C12N 15/11; C12N 15/90; C12Y 402/02003; C12Y 402/02011; C12N 2310/20; C12N 2795/00022; C12N 2795/00033

(57) ABSTRACT

Provided herein are bacteriophages engineered to express an exopolysaccharide (EPS) depolymerase for treating bacterial infections. In some embodiments, the EPS depolymerase comprises alginate lyase. Also envisioned within the scope of the invention are compositions comprising one or more of the bacteriophages, methods for treating bacterial infections, and kits comprising the compositions described herein.

### CROSS-REFERENCES TO RELATED APPLICATIONS

This application claims the benefit of U.S. Provisional Application No. 63/228,504, filed Aug. 2, 2021, which is incorporated herein by reference in its entirety and for all purposes.

### SEQUENCE LISTING

The instant application contains a Sequence Listing, which has been submitted electronically in ASCII format and is hereby incorporated by reference in its entirety. Said ASCII copy, created on Jul. 27, 2022 is named “054249-520001WO_SL_ST26” and is 3.51 megabytes in size.

### BACKGROUND

There is an increasing demand for alternative antibiotics as the number of bacterial strains resistant to traditional, small molecule antibiotic treatment regimens are becoming more numerous. Bacteriophage therapy uses bacterial viruses, or phages, to target and destroy bacteria at various sites of infection. Recent advances in biotechnology have allowed for the fast expansion of existing phage libraries in order to generate potent and specific phages that can target and destroy a bacterium of interest. Pseudomonas aeruginosa (PA) is an opportunistic pathogen that can potentially cause severe chronic and acute infections, especially in immune-compromised patients, with the potential for biofilm formation. Additionally, there are strains of PA that are antibiotic resistant, increasing the difficulty in treating these chronic infections. In some instances, PA infection may occur in the presence of cystic fibrosis (CF) or non-cystic fibrosis bronchiectasis (NCFB). Bacteriophage treatment approaches that can circumvent traditional mechanisms of antibiotic resistance, avoid the toxic side effects of traditional small molecule therapies, and can be effective against biofilms, are especially attractive.

### SUMMARY

Alginate, a major component of Pseudomonas aeruginosa biofilms, is a polysaccharide with two units: β-D-mannuronate (M) and α-L-guluronate (G). These units can be linked in homopolymers (polyG, polyM) or a heteropolymer (polyM/G). The overproduction of alginate (also referred to as “mucoidy”) is particularly prevalent in NCFB. Alginate lyases, which break down alginate, have been grouped into seven subfamilies of polysaccharide lyase (PL).

The activity of Sphingomonas A1-III from the PL5 family was previously explored. A1-III is known to break polyM alginate down into disaccharides and trisaccharides. When expressed in two phage families, A1-III was shown to have activity against preformed biofilms of mucoid Pseudomonas aeruginosa. The Alg2A version of alginate lyase, which belongs to the PL7 family and is expressed by Flavobacterium, was also expressed. Alg2A had previously been shown to degrade both polyG and polyM and showed greater activity against Pseudomonas aeruginosa lawns than A1-III. In addition, Alg2A enhanced antibiotic treatment of Pseudomonas aeruginosa biofilms. Therefore, Alg2A was cloned and expressed from phages to compare its activity to that of A1-III. The data described herein indicate that Alg2A has a stronger degradation effect on Pseudomonas aeruginosa biofilms than A1-III.

Described herein are bacteriophages, compositions of bacteriophages, combinations of phages, and use of the same for medical and non-medical applications, including in the treatment of bacterial infections and illnesses.

In one aspect, the present disclosure provides a bacteriophage engineered to express an exopolysaccharide (EPS) depolymerase.

In some embodiments, the EPS depolymerase is expressed from a nucleotide sequence selected from SEQ ID NO:20, SEQ ID NO:21, SEQ ID NO:22, SEQ ID NO:23, SEQ ID NO:24, SEQ ID NO:25, SEQ ID NO:36, or SEQ ID NO:59, or a sequence having at least 90% identity to the sequence of SEQ ID NO:20, at least 90% identity to the sequence of SEQ ID NO:21, at least 90% identity to the sequence of SEQ ID NO:22, at least 90% identity to the sequence of SEQ ID NO:23, at least 90% identity to the sequence of SEQ ID NO:24, at least 90% identity to the sequence of SEQ ID NO:25, at least 90% identity to the sequence of SEQ ID NO:36, or at least 90% identity to the sequence of SEQ ID NO:59.

In some embodiments, the EPS depolymerase is alginate lyase. In some embodiments, the alginate lyase comprises Alg2A or A1-III. In some embodiments, the alginate lyase comprises Alg2A. In some embodiments, the alginate lyase comprises A1-III.

In some embodiments, the bacteriophage shows improved host range.

In some embodiments, the bacteriophage belongs to the Genus Phikmvvirus. In some embodiments, the bacteriophage belongs to the Genus Pakpunavirus. In some embodiments, the bacteriophage belongs to the Genus Bruynoghevirus. In some embodiments, the bacteriophage belongs to the Genus Pbunavirus.

In some embodiments, the bacteriophage targets Pseudomonas aeruginosa. In some embodiments, the bacteriophage targets one or more of Pseudomonas aeruginosa, antibiotic-resistant Pseudomonas aeruginosa, and multiple antibiotic-resistant Pseudomonas aeruginosa. In some embodiments, the bacteriophage infects and kills one or more of Pseudomonas aeruginosa, antibiotic-resistant Pseudomonas aeruginosa, and multiple antibiotic-resistant Pseudomonas aeruginosa.

In some embodiments, the bacteriophage reduces biofilm mass.

In another aspect, the present disclosure provides a bacteriophage composition comprising one or more bacteriophages that express an exopolysaccharide (EPS) depolymerase, wherein the one or more bacteriophages comprise a polynucleotide sequence selected from SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO: 50; SEQ ID NO:51; SEQ ID NO:52; SEQ ID NO:53; SEQ ID NO:54; SEQ ID NO:55; SEQ ID NO:56; SEQ ID NO:57; SEQ ID NO:58; SEQ ID NO:60; SEQ ID NO:61; SEQ ID NO: 62; SEQ ID NO: 63; SEQ ID NO:64; SEQ ID NO:65; SEQ ID NO:66; SEQ ID NO:67; SEQ ID NO:68; SEQ ID NO:69; SEQ ID NO:70; SEQ ID NO:71, SEQ ID NO:73; a polynucleotide sequence with at least 90% identity to SEQ ID NO:26; a polynucleotide sequence with at least 90% identity to SEQ ID NO:27; a polynucleotide sequence with at least 90% identity to SEQ ID NO:28; a polynucleotide sequence with at least 90% identity to SEQ ID NO:29; a polynucleotide sequence with at least 90% identity to SEQ ID NO:30; a polynucleotide sequence with at least 90% identity to SEQ ID NO:31; a polynucleotide sequence with at least 90% identity to SEQ ID NO:32; a polynucleotide sequence with at least 90% identity to SEQ ID NO:33; a polynucleotide sequence with at least 90% identity to SEQ ID NO:34; a polynucleotide sequence with at least 90% identity to SEQ ID NO:35; a polynucleotide sequence with at least 90% identity to SEQ ID NO:37; a polynucleotide sequence with at least 90% identity to SEQ ID NO:38; a polynucleotide sequence with at least 90% identity to SEQ ID NO:39; a polynucleotide sequence with at least 90% identity to SEQ ID NO:40; a polynucleotide sequence with at least 90% identity to SEQ ID NO:41; a polynucleotide sequence with at least 90% identity to SEQ ID NO:42; a polynucleotide sequence with at least 90% identity to SEQ ID NO:43; a polynucleotide sequence with at least 90% identity to SEQ ID NO:44; a polynucleotide sequence with at least 90% identity to SEQ ID NO:45; a polynucleotide sequence with at least 90% identity to SEQ ID NO:46; a polynucleotide sequence with at least 90% identity to SEQ ID NO:47; a polynucleotide sequence with at least 90% identity to SEQ ID NO:48; a polynucleotide sequence with at least 90% identity to SEQ ID NO:49; a polynucleotide sequence with at least 90% identity to SEQ ID NO:50; a polynucleotide sequence with at least 90% identity to SEQ ID NO:51; a polynucleotide sequence with at least 90% identity to SEQ ID NO:52; a polynucleotide sequence with at least 90% identity to SEQ ID NO:53; a polynucleotide sequence with at least 90% identity to SEQ ID NO:54; a polynucleotide sequence with at least 90% identity to SEQ ID NO:55; a polynucleotide sequence with at least 90% identity to SEQ ID NO:56; a polynucleotide sequence with at least 90% identity to SEQ ID NO:57; a polynucleotide sequence with at least 90% identity to SEQ ID NO:58; a polynucleotide sequence with at least 90% identity to SEQ ID NO:60; a polynucleotide sequence with at least 90% identity to SEQ ID NO:61; a polynucleotide sequence with at least 90% identity to SEQ ID NO:62; a polynucleotide sequence with at least 90% identity to SEQ ID NO:63; a polynucleotide sequence with at least 90% identity to SEQ ID NO:64; a polynucleotide sequence with at least 90% identity to SEQ ID NO:65; a polynucleotide sequence with at least 90% identity to SEQ ID NO:66; a polynucleotide sequence with at least 90% identity to SEQ ID NO:67; a polynucleotide sequence with at least 90% identity to SEQ ID NO:68; a polynucleotide sequence with at least 90% identity to SEQ ID NO:69; a polynucleotide sequence with at least 90% identity to SEQ ID NO:70; a polynucleotide sequence with at least 90% identity to SEQ ID NO:71; a polynucleotide sequence with at least 90% identity to SEQ ID NO:73.

In some embodiments, the EPS depolymerase is alginate lyase.

In some embodiments, one or more of the bacteriophages are engineered. In some embodiments, two or more of the bacteriophages are engineered.

In some embodiments, a second bacteriophage of the one or more bacteriophages comprises a naturally occurring phage. In some embodiments, two or more bacteriophages of the one or more bacteriophages are naturally occurring phages.

In some embodiments, at least one of the bacteriophages target Pseudomonas aeruginosa.

In some embodiments, the one or more bacteriophages of the composition targets one or more of Pseudomonas aeruginosa, antibiotic-resistant Pseudomonas aeruginosa, and multiple antibiotic-resistant Pseudomonas aeruginosa.

In some embodiments, the one or more bacteriophages of the composition infect and kill one or more of Pseudomonas aeruginosa, antibiotic-resistant Pseudomonas aeruginosa, and multiple antibiotic-resistant Pseudomonas aeruginosa.

In some embodiments, the compositions further comprise a storage medium for storage at room temperature or a temperature at or below 8° C. In some embodiments, the composition is stored at a temperature ranging from −20° C. to 25° C. In some embodiments, the composition is stored at 2° C. to 8° C. In some embodiments, the composition is stored at room temperature. In some embodiments, the storage medium is for storage at 4° C., 0° C., −20° C., or −80° C.

In some embodiments, the storage medium comprises a cryoprotectant. In some embodiments, the cryoprotectant comprises glycerol. In some embodiments, the composition comprises between about 5% and about 50% glycerol. In some embodiments, the storage medium comprises about 20% glycerol. In some embodiments, the cryoprotectant comprises sucrose. In some embodiments, the composition comprises between about 5% and about 30% sucrose. In some embodiments, the composition comprises about 10% sucrose. In some embodiments, the cryoprotectant comprises dimethylsulfoxide (DMSO). In some embodiments, the DMSO is at a concentration of between 2% and 10%.

In some embodiments, the composition further comprises a pharmaceutically acceptable carrier, diluent, excipient or combinations thereof.

In some embodiments, the composition is a liquid, semi-liquid, solid, frozen, or lyophilized formulation.

In some embodiments, the composition comprises between 1×10⁸ and 1×10¹² PFU per milliliter of each bacteriophage.

In some embodiments, the one or more bacteriophages of the composition reduce biofilm mass.

In yet another aspect of the invention, provided herein is a method for treating a Pseudomonas aeruginosa infection, comprising administering any of the compositions described herein to a subject in need thereof.

In some embodiments, the composition is administered at a dosage of at least 3×10⁸ PFU of total bacteriophages per dose.

In some embodiments, the method further comprises administration of an antibiotic. In some embodiments, the antibiotic is selected from the group consisting of fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, quinolone, oxazolidinone, phenicol phosphonic acid, streptogramin, tetracycline, sulfonamide, imipenem, meropenem, amikacin, ciprofloxacin, levofloxacin, tobramycin, azithromycin, aztreonam, colistin, inhaled tobramycin, inhaled aztreonam, and inhaled colistin.

In some embodiments, the method further comprises administration of one or more CFTR modulators. In embodiments, the CFTR modulator may be selected from, but is not necessarily limited to, ivacaftor, lumacaftor/ivacaftor, tezacaftor/ivacaftor, elexacaftor/tezacaftor/ivacaftor, or any combination thereof.

In some embodiments, the bacterial infection has become resistant to one or more antibiotics selected from a fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, quinolone, oxazolidinone, phenicol phosphonic acid, streptogramin, tetracycline, sulfonamide, imipenem, meropenem, amikacin, ciprofloxacin, levofloxacin, tobramycin, azithromycin, aztreonam, colistin, inhaled tobramycin, inhaled aztreonam, and inhaled colistin.

In some embodiments, the bacteriophage composition is administered via inhalation. In some embodiments, the bacteriophage composition is administered via nebulization. In some embodiments, the bacteriophage composition is administered intravenously.

In some embodiments, the bacteriophage composition is administered at least once a day. In some embodiments, the bacteriophage composition is administered for at least one day.

In some embodiments, the subject is human.

In some embodiments, the subject suffers from cystic fibrosis (CF). In some embodiments, the subject suffers from non-cystic fibrosis bronchiectasis (NCFB).

In yet another aspect, the present disclosure provides an assay for determining alginate lyase activity of an engineered bacteriophage, comprising administering an effective amount of any of the engineered bacteriophages described herein to a Pseudomonas aeruginosa biofilm and determining reduction in biofilm mass.

In yet another aspect, the present disclosure provides a method for treating a bacterial infection comprising: (a) selecting a subject having a bacterial infection, and (b) administering to the subject an effective amount of any of the bacteriophages described herein, or any of the bacteriophage compositions described herein, thereby treating the bacterial infection. In some embodiments the subject is selected based upon having previously received a previous therapy for his/her bacterial infection. For example, the subject is selected based upon having already received at least one round of antibiotic treatment for the infection that did not completely resolve the infection or based upon having an infection by a bacterium that is resistant to one or more antibiotics.

In some embodiments, the bacterial infection is a Pseudomonas infection. In some embodiments, the bacterial infection is a Pseudomonas aeruginosa infection.

In some embodiments, the bacterial infection is characterized by a biofilm.

In some embodiments, the subject has cystic fibrosis (CF). In some embodiments, the subject has non-cystic fibrosis bronchiectasis (NCFB).

In some embodiments, the composition is administered at a dosage of at least 3×10⁸ PFU of total bacteriophages per dose.

In some embodiments, the method further comprises administration of an antibiotic. In some embodiments, the antibiotic is selected from the group consisting of fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, quinolone, oxazolidinone, phenicol phosphonic acid, streptogramin, tetracycline, sulfonamide, imipenem, meropenem, amikacin, ciprofloxacin, levofloxacin, tobramycin, azithromycin, aztreonam, colistin, inhaled tobramycin, inhaled aztreonam, and inhaled colistin.

In some embodiments, the method further comprises administration of one or more CFTR modulators. In embodiments, the CFTR modulator may be selected from, but is not necessarily limited to, ivacaftor, lumacaftor/ivacaftor, tezacaftor/ivacaftor, elexacaftor/tezacaftor/ivacaftor, or any combination thereof.

In some embodiments, the bacterial infection has become resistant to one or more antibiotics selected from a fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, quinolone, oxazolidinone, phenicol phosphonic acid, streptogramin, tetracycline, sulfonamide, imipenem, meropenem, amikacin, ciprofloxacin, levofloxacin, tobramycin, azithromycin, aztreonam, colistin, inhaled tobramycin, inhaled aztreonam, and inhaled colistin.

In some embodiments, the bacteriophage composition is administered via inhalation. In some embodiments, the bacteriophage composition is administered via nebulization.

In some embodiments, the bacteriophage composition is administered at least once a day. In some embodiments, the bacteriophage composition is administered for at least one day.

In some embodiments, the subject is human.

In yet another aspect, the present disclosure provides a method for making an engineered bacteriophage, comprising providing a bacteriophage and incorporating an exopolysaccharide (EPS) depolymerase into the bacteriophage.

In some embodiments, the EPS depolymerase is alginate lyase. In some embodiments, the alginate lyase comprises Alg2A or A1-III. In some embodiments, the alginate lyase comprises Alg2A. In some embodiments, the alginate lyase comprises A1-III.

In some embodiments, the bacteriophage belongs to the Genus Phikmvvirus. In some embodiments, the bacteriophage belongs to the Genus Pakpunavirus. In some embodiments, the bacteriophage belongs to the Genus Bruynoghevirus. In some embodiments, the bacteriophage belongs to the Genus Pbunavirus.

In yet another aspect, the present disclosure provides a kit comprising any of the bacteriophages described herein, or any of the bacteriophage compositions described herein, and instructions for using the same.

In some embodiments, the kit further comprises an antibiotic.

In some embodiments, the kit further comprises a means of administering the bacteriophage or bacteriophage composition. In some embodiments, the means comprises a syringe, a transdermal patch, a slow-release device, a spray, a nebulizer, an inhaler, or a respirator. In some embodiments, the slow-release device comprises a mini-osmotic pump.

In some embodiments, the kit further comprises a second bacteriophage or bacteriophage composition.

In yet another aspect, the present disclosure provides a bacteriophage composition comprising one or more bacteriophages engineered to express an exopolysaccharide (EPS) depolymerase, wherein the one or more bacteriophages belong to the Genus Phikmvvirus, Pakpunavirus, Bruynoghevirus, and/or Pbunavirus.

### BRIEF DESCRIPTION OF THE DRAWINGS

Embodiments will now be described, by way of example only, with reference to the accompanying drawings, in which:

FIG. 1 shows various degrees of bacterial clearance by different variants of an engineered bacteriophage of SEQ ID NO:2 on a preformed biofilm, compared to a saline control (PBS).

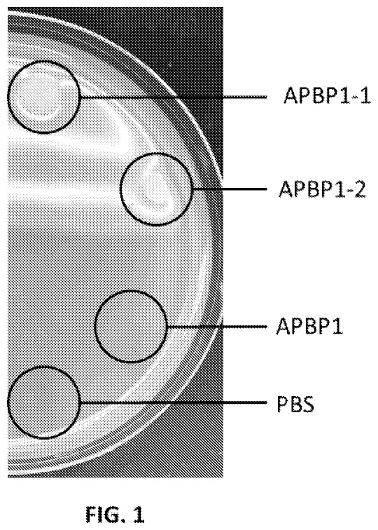

FIGS. 2A-2B show the alginate lyase activity displayed by various transgenic phages. FIG. 2A shows a top-lit view of phage spotted on preformed lawns of mucoid, alginate expressing strains of Pseudomonas aeruginosa (strain 15844) where activity presents as a crater or indentation lawn at the site of application. FIG. 2B shows a bottom-lit view of the same plate seen in 4A, where phage spotted on preformed lawns of mucoid, alginate expressing strains of Pseudomonas aeruginosa (strain 15844) can be seen as circular indentations on the lawn at the site of application.

FIG. 3 shows Western blotting detection of alginate lyase (AlgL) A1-III-His₆ in lysates of APBP1-1, APBP1-2, and APBP3-2.

FIG. 4 shows the position of the N-terminal fragment A1-III of Sphingomonas sp. Alginate lyase Aly and several variations of this fragment.

FIGS. 5A-5D show the difference in expression and activity between the A1-III fragments 70-399 and 54-412. FIG. 5A shows a Western blot of the 70-399 fragment, showing that it expresses poorly in E. coli. FIG. 5B shows Western blot of the 54-412 fragment, showing that it expresses well in E. coli. FIG. 5C shows activity of various clones on 2% seaweed alginate plates. A1-C7 is a clone with the correct sequence of A1-III with a C-terminal His tag. A1-C4 is a clone with a frameshift mutation resulting in no expression of A1-III. A1-III expression was performed by induction at mid-log after RT incubation (not shaking) for 30 min with 0.5 mM IPTG. A1-III-His₆ was purified from cell lysates using Ni-NTA affinity columns and spotted on a plate containing 2% alginate with commercial alginate lyase from Sigma-Aldrich as a control. FIG. 5D shows activity of the A1-III protein expressed and purified from clone A1-C7 on preformed lawns of mucoid, alginate-expressing strains of Pseudomonas aeruginosa (strain 15840). AL10 is 10 ng of alginate lyase purchased from Sigma-Aldrich as control, A1-III 10 is 10 ng of purified A1-III. 50, 1, 0.5 and 0.1 refer to the amount of A1-III spotted in ng.

FIGS. 6A-6D show that alginate lyase A1-III is expressed as a fusion protein with gp13 when engineered downstream of APBP4 gp13.1. FIG. 6A shows a Western blot of lysates of engineered phages carrying the A1-III alginate lyase payload (left) and a table identifying the contents of each lane (right). Box indicates bands corresponding to the fusion protein. FIG. 6B shows the DNA sequence around the C-terminus of A1-III illustrating how trans-reading the TGA stop codon of A1-III-His₆ could lead to an in-frame fusion protein with gp13 through a 19 aa-long “anti-terminated linker”. FIG. 6C shows the putative sequence of the A1-III-His₆-linker-gp13 fusion protein generated in lysates of APBP4-4. Residues in bold correspond to the A1-III-His₆ sequence, underlined resides correspond to gp13, grey residues denote the putative linker and the black highlighted tryptophan residue is a likely way for the cell to misread a TGA stop codon of A1-III-His₆. FIG. 6D illustrates that APBP4-4 shows no activity on a mucoid lawn of alginate-producing Pseudomonas aeruginosa strain 15844, indicating the fusion protein is non-functional.

FIG. 7 shows Western blot expression of lysates of engineered phages carrying the A1-III alginate lyase payload (left) and a table identifying the contents of each lane (right).

FIGS. 8A-8B show results from cloning two codon usage matrices for alginate lyase A1-III into phages. FIG. 8A shows a sequence alignment between the two different codon usage matrices. FIG. 8B shows alginate lyase activity of phages expressing the respective payloads on a mucoid lawn of P. aeruginosa 15844.

FIGS. 9A-9I show examples of alginate lyase activity profiles from lysates of engineered phages expressing various configurations of A1-III; FIG. 9A shows activity of A1-III⁷⁰⁻³⁹⁹ engineered into APBP4-1, FIG. 9B shows activity of A1-III⁷⁰⁻³⁹⁹ engineered into APBP17-1, FIG. 9C shows activity of A1-III⁵⁴⁻⁴⁰⁸ engineered into APBP1-4, FIG. 9D shows activity of A1-III⁵⁴⁻⁴⁰⁸ engineered into APBP4-5, FIG. 9E shows activity of A1-III⁵⁴⁻⁴¹²-His₆ engineered into APBP18-1, FIG. 9F shows activity of A1-III⁵⁴⁻⁴¹²-His₆ engineered into APBP1-2, FIG. 9G shows activity of A1-III⁵⁴⁻⁴¹²-His₆ engineered into APBP3-6, FIG. 9H shows activity of A1-III⁵⁴⁻⁴¹²-His₆ engineered into APBP1-1, and FIG. 9I shows activity of A1-III⁵⁴⁻⁴¹²-His₆ engineered into APBP3-2.

FIGS. 10A-10E show that Flavobacterium Alg2A (accession number AEB69783) expressed from multiple phages has alginate lyase activity whether it retains its N-terminal signal sequence, is tagged with a C-terminal His₆-tag, or is encoded by 4 different genes. FIG. 10A shows an alignment of different versions of Alg2A protein cloned in a variety of phages. FIG. 10B shows alginate lyase activity by phages expressing the full-length Alg2A with a C-terminus His tag on mucoid lawns of alginate-producing Pseudomonas aeruginosa strain 15844. FIG. 10C shows alginate lyase activity of phages expressing the shorter signal sequence-deleted version of Alg2A²³⁻²⁸⁸ without a His₆-tag. FIG. 10D illustrates the percent identity between the different genes coding for the Alg2A²³⁻²⁸⁸ protein and cloned in APBP3. FIG. 10E shows alginate lyase activity of APBP3-derived phages expressing Alg2A²³⁻²⁸⁸ from SEQ ID NO:37, SEQ ID NO:38, or SEQ ID NO:39 on mucoid lawns of alginate-producing Pseudomonas aeruginosa strain 15844.

FIG. 11 shows alginate lyase activity on a preformed mucoid lawn by a cocktail of phages engineered with either A1-III or Alg2A.

FIG. 12 shows that growth of phages APBP1-1, APBP1-2, APBP3-1, and APBP3-2 is not altered by expression of alginate lyase protein A1-III.

FIGS. 13A-13F shows results from a host range improvement assay using different bacteriophages with or without alginate lyase activity. Phage dilution increases 10-fold in each spot from top to bottom. FIG. 13A illustrates that phages all show the same titer on their host, Pseudomonas aeruginosa 7299. FIG. 13B On Pseudomonas aeruginosa strain PS 30, APBP3-1 shows clearing down to the 10⁻² dilution while its parent APBP3 does not plaque at all on this strain. Similarly, APBP1-1 and APBP1-2 plaque on the host (Pseudomonas aeruginosa strain PS 30), while their parent APBP1 does not. FIG. 13C APBP1-1 and APBP1-2 show improved clearing on the Pseudomonas aeruginosa strain 7176, compared to their parent APBP1. FIG. 13D The apparent titer of APBP1-1 and APBP1-2 is ˜2⁸ PFU/ml on Pseudomonas aeruginosa strain 15843, while the apparent titer of APBP1 (their parent) is only around ten-fold lower at ˜2⁷ PFU/ml. FIG. 13E On Pseudomonas aeruginosa strain 15839, APBP3-1 shows clearing while its parent APBP3 does not plaque at all on this strain. APBP1-1 and APBP1-2 also produce clearings on the host while their parent APBP1 does not. FIG. 13F APBP3-1 shows increased clearing compared to parent APBP3 on this Pseudomonas aeruginosa strain 15840. APBP1-1 and APBP1-2 also show improved clearing compared to APBP1.

FIG. 14 is a graph showing the ability of different phage strains expressing no lyase (WT), A1-III (Eng-A1-III) or Alg2A (Eng-Alg2A) to disrupt biofilms.

FIGS. 15A-15D illustrate the suitability of various loci in the APBP6 phage genome for engineering of A1-III. FIG. 15A Representation of the recombination between pLIX36 and APBP6 to integrate A1-III between gp038 and gp039. FIG. 15B Representation of the recombination between pLIX46 and APBP6 to integrate A1-III between gp005 and gp006. FIG. 15C Agarose gel showing insertion of A1-III in APBP6, when grown on a pLIX36-containing strain (left) then passaged on a strain without (right). FIG. 15D Agarose gel showing insertion of A1-III in APBP6, when grown on a pLIX46-containing strain (left) then passaged on a strain without (right).

FIG. 16 is a Western blot showing that engineered phages ABP4-6, ABP18-2, ABP6-3 express alginate lyase protein Alg2A²³⁻²⁸⁸ (left) and a table identifying the contents of each lane (right).

FIG. 17 is a Western blot showing that engineered phages APBP3-5 and APBP1-5 express alginate lyase protein Alg2A¹⁻²⁸⁸ and Alg2A²³⁻²⁸⁸, respectively (left) and a table identifying the contents of each lane (right).

FIG. 18 is a Western blot of lysates of engineered phages carrying the A1-III⁵⁴⁻⁴¹² or A-III⁵⁴⁻⁴⁰⁸ alginate lyase gene (left) and a table identifying the contents of each lane (right).

### DETAILED DESCRIPTION

As noted above, there is an antibiotic crisis in the world. Bacterial illness is an ever-present concern, while increasing antibiotic resistance means the number of available and effective antibiotics continues to shrink. The embodiments and aspects of this application provide exciting alternative solutions to the use of standard antibiotics. These embodiments and inventions are the result of significant, non-trivial inventive effort, and the solving of technical challenges and hurdles.

As a result, embodiments and aspects described herein generally relate to novel and inventive bacteriophages, for example, effective for treating Pseudomonas infections, alone or in combinations. Described are methods of treating Pseudomonas bacterial infections generally, but also certain types of infections, for example, respiratory infections, infections associated with fibrosis, pneumonia, etc. Storage and manufacturing compositions and methods are described. The various embodiments and aspects present exciting and critically needed solutions for the antibiotic crisis across the world.

It is to be understood that the present disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

The detailed description of the present disclosure is divided into various sections only for the reader's convenience and disclosure found in any section may be combined with that in another section. Unless defined otherwise, all technical and scientific terms used herein have the same meaning as commonly understood by one of ordinary skill in the art to which the present disclosure belongs.

### Definitions

It must be noted that as used herein and in the appended claims, the singular forms “a,” “an,” and “the” include plural referents unless the context clearly dictates otherwise. Thus, for example, reference to “a bacteriophage composition” includes a plurality of such candidate agents and reference to “the bacteriophage” includes reference to one or more bacteriophages and equivalents thereof known to those skilled in the art, and so forth.

In this disclosure, “comprises,” “comprising,” “containing” and “having” and the like can have the meaning ascribed to them in U.S. Patent law and can mean “includes,” “including,” and the like. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. “Consisting of” shall mean excluding more than trace elements of other ingredients and substantial method steps. Embodiments defined by each of these transition terms are within the scope of this disclosure.

The term “consists essentially of” as used herein means that only the bacteriophage(s) explicitly indicated are present in the bacteriophage composition, but that said composition may also contain a further non-bacteriophage constituent, such as a pharmaceutically appropriate carrier, diluent, excipient, antibiotic (e.g., chemical antibiotic), etc., or combinations thereof.

As used herein, the term “about” when used before a numerical designation, e.g., temperature, time, amount, concentration, and such other, including a range, indicates approximations which may vary by (+) or (−) 10%, 5%, or 1%. All values in this disclosure are preceded by the term “about,” even if not explicitly recited.

When a range (e.g., dosage range) is listed herein, it is to be understood that the value may include any individual value or range within the recited range(s), including endpoints.

As used herein, the terms “mutant” and “variant” are used interchangeably, and refer to a bacteriophage differing genetically from a reference bacteriophage, but that still retains the ability to infect and kill target bacteria, e.g., Pseudomonas aeruginosa. For example, “mutant” can refer to a bacteriophage that has mutated genetically compared to one or more of SEQ ID NO:1 to SEQ ID NO:11 and/or any of the other bacteriophage referenced or described herein, but that still retains the ability to infect and kill Pseudomonas aeruginosa target bacteria. Mutants can comprise e.g., silent mutations, conservative mutations, minor deletions, and/or minor replications of genetic material, and retain phenotypic characteristics of the reference bacteriophage. In embodiments, a “mutant” may be a bacteriophage progeny. A bacteriophage progeny may be a bacteriophage obtainable after lysing Pseudomonas (e.g., P. aeruginosa) target bacteria using a bacteriophage as described herein (i.e., the “parent bacteriophage”). In other words, the bacteriophage progeny may be a second (or further) generation bacteriophage. In an embodiment, the mutants retain any observable characteristic or property that is dependent upon the genome of the bacteriophage as described herein, i.e. phenotypic characteristics of said bacteriophage and/or lytic activity against Pseudomonas species or strains. Preferred mutants retain the ability to infect and kill Pseudomonas aeruginosa target bacteria and have less than 10% nucleic acid variation as compared to the genome of the reference bacteriophage, even more preferably less than 7%, more preferably less than 1%. Alternatively, or in combination, mutants have preferably less than 7% amino acid variation in a coded polypeptide sequence as compared to a polypeptide of the reference bacteriophage.

As used herein, the terms “% identity,” “% sequence identity” and “percent identity” in relation to nucleic acid or amino acid sequences designates the level of identity or homology between said sequences and may be determined by techniques known in the art. Any of a variety of sequence alignment methods can be used to determine percent identity, including, without limitation, global methods, local methods and hybrid methods, such as segment approach methods. Protocols to determine percent identity are routine procedures within the scope of one skilled in the art. Global methods align sequences from the beginning to the end of the molecule and determine the best alignment by adding up scores of individual nucleotide pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement. Non-limiting methods include, e.g., BLAST, Match-box, see, e.g., Align-M, see, e.g., Ivo Van Walle et al., Align-M—A New Algorithm for Multiple Alignment of Highly Divergent Sequences, Bioinformatics 20(9):1428-1435 (2004). This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 100 nucleotides in length, or more preferably over a region that is 100-1000 or more nucleotides in length.

As used herein, the terms “treating” or “treatment” (and as well understood in the art) are used in accordance with their plain and ordinary meaning and broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things. As used herein, the term “treat” or “treating” is intended to encompass prophylactic treatment as well as corrective treatment (treatment of a subject already suffering from a disease).

As used herein, the term “administering” means oral, intravenous, parenteral, intraperitoneal, intramuscular, intrathecal, intranasal, pulmonary, or subcutaneous administration for example, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, and the like. In embodiments, the administering does not include administration of any active agent other than the recited active agent. In embodiments, administration of compositions described herein is by intravenous administration. In embodiments, administration of compositions described herein is by intranasal administration such as inhalation or nebulization. In embodiments, administration may be pulmonary delivery via nasal or oral administration (e.g. by aerosolization or nebulization).

“Co-administer” it is meant that a composition described herein is administered at the same time, just prior to, or just after the administration of one or more additional therapies. The compounds provided herein can be administered alone or can be co-administered to the patient. Co-administration is meant to include simultaneous or sequential administration of the compounds individually or in combination (more than one compound). Thus, the preparations can also be combined, when desired, with other active substances (e.g. antibiotic).

As used herein, the term “lytic” or “lytic activity” designates the property of a bacteriophage to cause lysis of a bacterial cell. The lytic activity of a bacteriophage can be tested on a bacterium (e.g., P. aeruginosa strains) according to techniques known in the art. The lytic cycle is named for the process that occurs when a phage has infected a cell, replicated new phage particles, and bursts through the host cell membrane. Some phage exhibit a lysogenic cycle during which the bacteriophage DNA remains practically dormant due to active repression of bacteriophage processes. Whenever the bacterium divides, the DNA of the phage is copied as well. In this way, the virus can continue replicating within its host without lysing the host. At a certain point, conditions may change and the phage enters a lytic cycle. “Obligately lytic” refers to phage that are unable to undergo a lysogenic cycle.

As used herein, the term “bacteriophage target” refers to any bacteria species that can be infected by a particular bacteriophage. A bacteriophage recognizes the target bacterial cell surface, binds, and injects its genetic material inside the bacterial host. The genetic material from the infecting phage can be incorporated into the bacterial genome. The bacteriophage may become lysogenic, where the viral genome remains dormant in the bacterial host genome until a triggering event. The bacteriophage may also become lytic, wherein many copies of the infecting phage are produced by the machinery of the infected bacteria, and the copies are subsequently released by bacterial lysis, extrusion, or by budding. In embodiments, the bacterial target is Pseudomonas aeruginosa.

As used herein, the term “bacterial host manufacturing strain” or “manufacturing strain” refers to the bacteria used to grow bacteriophage. A method for bacteriophage production may require a production process involving at least two operating units, growth of the host bacteria and bacteriophage propagation (or infection). It is important to consider basic parameters for bacterial growth and phage infection, such as the selected substrates for the bacterium and the optimal temperature, both for bacterial growth and phage infection, since these factors may influence the infectivity of phages.

A use or method typically comprises administering a bacteriophage or bacteriophage composition described herein to a subject. As used herein, the term “subject” or “patient” refers to a human or non-human animal. Preferably, the subject is a human. Preferably, the subject or patient is in need of treatment with the composition as described herein, e.g., has a bacterial infection susceptible to treatment with the composition.

As used herein, the term “isolated” indicates that the bacteriophage are removed from its original environment in which it naturally occurs. In particular, an isolated bacteriophage is, e.g., cultivated, cultured separately from the environment in which it is naturally located.

As used herein, the term “purified” indicates that the bacteriophages are removed from nature and/or a manufacturing host bacteria. In particular, a purified bacteriophage has production impurities, such as bacterial components, substantially removed from its manufacturing or production environment. Bacterial components include but are not limited to bacterial host proteins, lipids, and/or bacterial endotoxin. The term “purified” may also refer to genetic purification in which the strain of bacteriophage is genetically homogenous. In some embodiments, the purified bacteriophage comprises a bacteriophage that is at least 99% pure, or at least 99% of the desired population of bacteriophages.

As used herein, the term “substantially purified” refers to a composition containing less than 1%, less than 0.1%, less than 0.001%, or no detectable amount of contaminants such as host bacterial proteins or endotoxin. Also, as used herein, the term “substantially pure” when used to describe a bacteriophage strain refers to the genetic purity of the composition such that the strain is greater than 99%, greater than 99.9%, greater than 99.999%, or 100% of one particular genome sequence.

Typically, a composition is substantially pure when at least 80%, at least 85%, at least 90%, at least 95%, at least 98%, or at least 99% (or any sub value or subrange therebetween) of the total material (by volume, by wet or dry weight, or by mole percent or mole fraction) in a sample is free of impurities or genetic variants.

As used herein, the “synergistic amount” refers to the sum of a first amount (e.g., a bacteriophage) and a second amount (e.g., a different bacteriophage) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent.

As used herein, the term “substantially free” refers to something having less than 10% of the substance that it is to be free from. For example, 0.01% to 10% free of the substance, including any subvalue and subrange therein, including endpoints. For example, 0.01, 0.02, 0.03, 0.04, 0.05, 0.06, 0.07, 0.08, 0.09, 1, 2, 3, 4, 5, 6, 7, 8, 9, or 10% (or any sub value or subrange therebetween, inclusive of endpoints).

As used herein, the term “obtainable” as used herein also encompasses the term “obtained.” In one embodiment, the term “obtainable” means obtained.

As used herein, the terms “pharmaceutically acceptable excipient,” “pharmaceutically acceptable diluent,” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

As used herein, the term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like.

As used herein, the term “persist” refers to the ability to remain present or continue to exist past a usual, expected, or normal time.

As used herein, “improved,” “broadened” or “broader” in the context of bacteriophage target range refers to increased host range. Host range is the number of cell types, strains, or host species a virus/bacteriophage (or combination of viruses) is able to infect. Increase of host range or target bacteria range is an expansion of the absolute number of distinct cell types, strains, or species a virus (or combination of viruses) is able to infect compared to a reference and/or non-engineered virus. In some examples, increased host range or increased target bacteria range is an increase in the number of bacterial strains or variants within a bacterial species that the virus (or combination of viruses) is able to infect. The increase in host range can be an increase of at least one or more than one strain, cell type, or species. Host range can be assayed, for example, by a standard plaque assay that is well known in the art.

As used herein, “multiplicity of infection (MOI)” is the ratio of the numbers of virus particles to the numbers of the host cells in a given infection medium. A value of MOI=1 implies that on an average there is a single host cell for a single phage particle.

As used herein, “partially synthetic” phage refers to a phage for which a limited, fractional, or substantial portion of the genome has been designed or engineered. As used herein, “fully synthetic” phage refers to a phage for which the entire genome has been designed or engineered.

Additional terms and phrases are defined below.

### Engineered Bacteriophages

In some embodiments, the present disclosure relates to one or more bacteriophages engineered to express a heterologous gene. In some embodiments, the heterologous gene may comprise an exopolysaccharide (EPS) depolymerase, an enzyme that breaks down a polysaccharide into smaller fragments. Bacteriophages of the Podoviridae family often exhibit so-called depolymerases as structural components of the virion. These enzymes appear as tail spike proteins. After specific binding to capsular polysaccharides, exopolysaccharides (EPS) or lipopolysaccharide (LPS) of the host bacteria, polysaccharide-repeating units are specifically cleaved. Finally, the phage reaches the outer membrane, deploys machinery that allows it to inject its DNA across both membranes and the cell wall, and infects the cell.

A depolymerase is a structural component of the adsorption apparatus, which facilitates binding and digestion of capsules. The name indicates that the repeating unit of a polysaccharide is cleaved and disintegrated. Biochemically, depolymerases are divided into two groups, lyases and hydrolases. Lyases, in contrast to hydrolases, cleave their substrates non-hydrolytically, meaning that no water molecule is released after substrate cleaving. Most of the well-characterized phage encoded depolymerases, which target EPS or LPS 0-polysaccharides, are lyases (Tomlinson and Taylor, 1985; Linnerborg et al., 2001; Olszak et al., 2017). They generally feature great diversity in substrate specificity. However, a particular cleavage site may be present in different polysaccharide types, thereby allowing the enzyme to act on two different substrates. The term depolymerase can refer to any generic protein that is able to degrade polymers. From this perspective, phage encoded endolysins are also depolymerases, since they cleave the peptidoglycan, a bacterial polysaccharide in general in a hydrolase manner (Schmelcher and Loessner, 2016).

In some embodiments, the EPS depolymerase comprises alginate lyase. Alginate is a linear polysaccharide that has been isolated from a variety of organisms, ranging from plants and bacteria to fungi. It is also a major component of the cell wall in brown algae and a major source of fixed carbon for other organisms. Alginate lyase belongs to the family of lyases, specifically those carbon-oxygen lyases acting on polysaccharides, whereby it catalyzes the degradation of alginate into various monosaccharide and polysaccharide products. The systematic name of this enzyme class is poly(beta-D-1,4-mannuronide) lyase. Other names in common use include, without limitation, alginate lyase I, alginate lyase, alginase I, alginase II, alginase, and alginate lyase III. This enzyme participates in fructose and mannose metabolism.

In some embodiments, the present disclosure provides bacteriophages engineered to express Alg2A. In some embodiments, the present disclosure provides bacteriophages engineered to express A1-III. In some embodiments, the EPS depolymerase sequences described herein may include portions of, or functional fragments of an EPS depolymerase gene. In some embodiments, they may include a whole EPS depolymerase gene.

As used herein, the terms “functional fragment” or “functional variant” refer to a molecule, including a nucleic acid or protein, for example, that comprises a nucleotide and/or amino acid sequence that is altered by one or more nucleotides and/or amino acids compared to the nucleotide and/or amino acid sequences of the parent or reference molecule. For a protein, a functional variant is still able to function in a manner that is similar to the parent molecule. In other words, the modifications in the amino acid and/or nucleotide sequence of the parent molecule do not significantly affect or alter the functional characteristics of the molecule encoded by the nucleotide sequence or containing the amino acid sequence. The functional variant may have conservative sequence modifications including nucleotide and amino acid substitutions, additions and deletions. These modifications can be introduced by standard techniques known in the art, such as direct DNA synthesis, site-directed mutagenesis and random PCR-mediated mutagenesis. Functional variants can also include, but are not limited to, derivatives that are substantially similar in primary structural sequence, but which contain, e.g., in vitro or in vivo modifications, chemical and/or biochemical, that are not found in the parent molecule. Such modifications include, inter alia, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI-anchor formation, hydroxylation, iodination, methylation, myristoylation, oxidation, pegylation, proteolytic processing, phosphorylation, prenylation, racemization, selenoylation, sulfation, transfer-RNA-mediated addition of amino acids to proteins such as arginylation, ubiquitination, and the like.

The EPS depolymerase may be from any organism that expresses an EPS depolymerase. In some embodiments, the EPS depolymerase may originate from bacterial genera selected from, but not necessarily limited to members of Sphingomonas, Flavobacterium, Pseudomonas, Klebsiella, Corynebacterium, Alteromonas, Zobelia, Aplysia, Vibrio, Saccharophagus, Stenotrophomonas, Streptomyces, Shewanella, Agrobacterium, and/or Azotobacter. Specifically, the EPS depolymerase may originate from, but is not necessarily limited to, Sphingomonas (accession no. BAB03312), Flavobacterium (accession no. AEB69783), Pseudomonas (accession no. 1VAV), Klebsiella (accession no. 40ZX), Corynebacterium (accession no. 1UAI), Alteromonas (accession no. 1J1T), Zobelia (accession nos. 3ZPY, 4BE3), Pseudoalteromonas (accession no. 4Q8K), Aplysia (accession no. 5GMT), Vibrio (accession no. WP_017072010.1), Saccharophagus (accession no. WP_011469755.1), Stenotrophomonas (accession no. WP_049467230.1), Streptomyces (accession no. NED67686.1), Shewanella (accession no. WP_188926150.1), Agrobacterium (accession no. WP_046801053.1), Azotobacter (accession no. Q9ZFG9).

In some embodiments, the EPS depolymerase is encoded by the nucleotide sequence of SEQ ID NO:20. In some embodiments, the EPS depolymerase is encoded by a sequence having at least 90% identity to the sequence of SEQ ID NO:20. In some embodiments, the EPS depolymerase is encoded in a sequence having at least 91% identity to the sequence of SEQ ID NO:20. In some embodiments, the EPS depolymerase is encoded in a sequence having at least 92% identity to the sequence of SEQ ID NO:20. In some embodiments, the EPS depolymerase is encoded in a sequence having at least 93% identity to the sequence of SEQ ID NO:20. In some embodiments, the EPS depolymerase is encoded in a sequence having at least 94% identity to the sequence of SEQ ID NO:20. In some embodiments, the EPS depolymerase is encoded in a sequence having at least 95% identity to the sequence of SEQ ID NO:20. In some embodiments, the EPS depolymerase is encoded in a sequence having at least 96% identity to the sequence of SEQ ID NO:20. In some embodiments, the EPS depolymerase is encoded in a sequence having at least 97% identity to the sequence of SEQ ID NO:20. In some embodiments, the EPS depolymerase is encoded in a sequence having at least 98% identity to the sequence of SEQ ID NO:20. In some embodiments, the EPS depolymerase is encoded in a sequence having at least 99% identity to the sequence of SEQ ID NO:20.

In some embodiments, the EPS depolymerase is encoded by the nucleotide sequence of SEQ ID NO:21. In some embodiments, the EPS depolymerase is encoded by a sequence having at least 90% identity to the sequence of SEQ ID NO:21. In some embodiments, the EPS depolymerase is encoded in a sequence having at least 91% identity to the sequence of SEQ ID NO:21. In some embodiments, the EPS depolymerase is encoded in a sequence having at least 92% identity to the sequence of SEQ ID NO:21. In some embodiments, the EPS depolymerase is encoded in a sequence having at least 93% identity to the sequence of SEQ ID NO:21. In some embodiments, the EPS depolymerase is encoded in a sequence having at least 94% identity to the sequence of SEQ ID NO:21. In some embodiments, the EPS depolymerase is encoded in a sequence having at least 95% identity to the sequence of SEQ ID NO:21. In some embodiments, the EPS depolymerase is encoded in a sequence having at least 96% identity to the sequence of SEQ ID NO:21. In some embodiments, the EPS depolymerase is encoded in a sequence having at least 97% identity to the sequence of SEQ ID NO:21. In some embodiments, the EPS depolymerase is encoded in a sequence having at least 98% identity to the sequence of SEQ ID NO:21. In some embodiments, the EPS depolymerase is encoded in a sequence having at least 99% identity to the sequence of SEQ ID NO:21.

In some embodiments, the EPS depolymerase is encoded by the nucleotide sequence of SEQ ID NO:22. In some embodiments, the EPS depolymerase is encoded by a sequence having at least 90% identity to the sequence of SEQ ID NO:22. In some embodiments, the EPS depolymerase is encoded in a sequence having at least 91% identity to the sequence of SEQ ID NO:22. In some embodiments, the EPS depolymerase is encoded in a sequence having at least 92% identity to the sequence of SEQ ID NO:22. In some embodiments, the EPS depolymerase is encoded in a sequence having at least 93% identity to the sequence of SEQ ID NO:22. In some embodiments, the EPS depolymerase is encoded in a sequence having at least 94% identity to the sequence of SEQ ID NO:22. In some embodiments, the EPS depolymerase is encoded in a sequence having at least 95% identity to the sequence of SEQ ID NO:22. In some embodiments, the EPS depolymerase is encoded in a sequence having at least 96% identity to the sequence of SEQ ID NO:22. In some embodiments, the EPS depolymerase is encoded in a sequence having at least 97% identity to the sequence of SEQ ID NO:22. In some embodiments, the EPS depolymerase is encoded in a sequence having at least 98% identity to the sequence of SEQ ID NO:22. In some embodiments, the EPS depolymerase is encoded in a sequence having at least 99% identity to the sequence of SEQ ID NO:22.

In some embodiments, the EPS depolymerase is encoded by the nucleotide sequence of SEQ ID NO:23. In some embodiments, the EPS depolymerase is encoded by a sequence having at least 90% identity to the sequence of SEQ ID NO:23. In some embodiments, the EPS depolymerase is encoded in a sequence having at least 91% identity to the sequence of SEQ ID NO:23. In some embodiments, the EPS depolymerase is encoded in a sequence having at least 92% identity to the sequence of SEQ ID NO:23. In some embodiments, the EPS depolymerase is encoded in a sequence having at least 93% identity to the sequence of SEQ ID NO:23. In some embodiments, the EPS depolymerase is encoded in a sequence having at least 94% identity to the sequence of SEQ ID NO:23. In some embodiments, the EPS depolymerase is encoded in a sequence having at least 95% identity to the sequence of SEQ ID NO:23. In some embodiments, the EPS depolymerase is encoded in a sequence having at least 96% identity to the sequence of SEQ ID NO:23. In some embodiments, the EPS depolymerase is encoded in a sequence having at least 97% identity to the sequence of SEQ ID NO:23. In some embodiments, the EPS depolymerase is encoded in a sequence having at least 98% identity to the sequence of SEQ ID NO:23. In some embodiments, the EPS depolymerase is encoded in a sequence having at least 99% identity to the sequence of SEQ ID NO:23.

In some embodiments, the EPS depolymerase is encoded by the nucleotide sequence of SEQ ID NO:24. In some embodiments, the EPS depolymerase is encoded by a sequence having at least 90% identity to the sequence of SEQ ID NO:24. In some embodiments, the EPS depolymerase is encoded in a sequence having at least 91% identity to the sequence of SEQ ID NO:24. In some embodiments, the EPS depolymerase is encoded in a sequence having at least 92% identity to the sequence of SEQ ID NO:24. In some embodiments, the EPS depolymerase is encoded in a sequence having at least 93% identity to the sequence of SEQ ID NO:24. In some embodiments, the EPS depolymerase is encoded in a sequence having at least 94% identity to the sequence of SEQ ID NO:24. In some embodiments, the EPS depolymerase is encoded in a sequence having at least 95% identity to the sequence of SEQ ID NO:24. In some embodiments, the EPS depolymerase is encoded in a sequence having at least 96% identity to the sequence of SEQ ID NO:24. In some embodiments, the EPS depolymerase is encoded in a sequence having at least 97% identity to the sequence of SEQ ID NO:24. In some embodiments, the EPS depolymerase is encoded in a sequence having at least 98% identity to the sequence of SEQ ID NO:24. In some embodiments, the EPS depolymerase is encoded in a sequence having at least 99% identity to the sequence of SEQ ID NO:24.

In some embodiments, the EPS depolymerase is encoded by the nucleotide sequence of SEQ ID NO:25. In some embodiments, the EPS depolymerase is encoded by a sequence having at least 90% identity to the sequence of SEQ ID NO:25. In some embodiments, the EPS depolymerase is encoded in a sequence having at least 91% identity to the sequence of SEQ ID NO:25. In some embodiments, the EPS depolymerase is encoded in a sequence having at least 92% identity to the sequence of SEQ ID NO:25. In some embodiments, the EPS depolymerase is encoded in a sequence having at least 93% identity to the sequence of SEQ ID NO:25. In some embodiments, the EPS depolymerase is encoded in a sequence having at least 94% identity to the sequence of SEQ ID NO:25. In some embodiments, the EPS depolymerase is encoded in a sequence having at least 95% identity to the sequence of SEQ ID NO:25. In some embodiments, the EPS depolymerase is encoded in a sequence having at least 96% identity to the sequence of SEQ ID NO:25. In some embodiments, the EPS depolymerase is encoded in a sequence having at least 97% identity to the sequence of SEQ ID NO:25. In some embodiments, the EPS depolymerase is encoded in a sequence having at least 98% identity to the sequence of SEQ ID NO:25. In some embodiments, the EPS depolymerase is encoded in a sequence having at least 99% identity to the sequence of SEQ ID NO:25.

In some embodiments, the EPS depolymerase is encoded by the nucleotide sequence of SEQ ID NO:36. In some embodiments, the EPS depolymerase is encoded by a sequence having at least 90% identity to the sequence of SEQ ID NO:36. In some embodiments, the EPS depolymerase is encoded in a sequence having at least 91% identity to the sequence of SEQ ID NO:36. In some embodiments, the EPS depolymerase is encoded in a sequence having at least 92% identity to the sequence of SEQ ID NO:36. In some embodiments, the EPS depolymerase is encoded in a sequence having at least 93% identity to the sequence of SEQ ID NO:36. In some embodiments, the EPS depolymerase is encoded in a sequence having at least 94% identity to the sequence of SEQ ID NO:36. In some embodiments, the EPS depolymerase is encoded in a sequence having at least 95% identity to the sequence of SEQ ID NO:36. In some embodiments, the EPS depolymerase is encoded in a sequence having at least 96% identity to the sequence of SEQ ID NO:36. In some embodiments, the EPS depolymerase is encoded in a sequence having at least 97% identity to the sequence of SEQ ID NO:36. In some embodiments, the EPS depolymerase is encoded in a sequence having at least 98% identity to the sequence of SEQ ID NO:36. In some embodiments, the EPS depolymerase is encoded in a sequence having at least 99% identity to the sequence of SEQ ID NO:36.

In some embodiments, the EPS depolymerase is encoded by the nucleotide sequence of SEQ ID NO:59. In some embodiments, the EPS depolymerase is encoded by a sequence having at least 90% identity to the sequence of SEQ ID NO:59. In some embodiments, the EPS depolymerase is encoded in a sequence having at least 91% identity to the sequence of SEQ ID NO:59. In some embodiments, the EPS depolymerase is encoded in a sequence having at least 92% identity to the sequence of SEQ ID NO:59. In some embodiments, the EPS depolymerase is encoded in a sequence having at least 93% identity to the sequence of SEQ ID NO:59. In some embodiments, the EPS depolymerase is encoded in a sequence having at least 94% identity to the sequence of SEQ ID NO:59. In some embodiments, the EPS depolymerase is encoded in a sequence having at least 95% identity to the sequence of SEQ ID NO:59. In some embodiments, the EPS depolymerase is encoded in a sequence having at least 96% identity to the sequence of SEQ ID NO:59. In some embodiments, the EPS depolymerase is encoded in a sequence having at least 97% identity to the sequence of SEQ ID NO:59. In some embodiments, the EPS depolymerase is encoded in a sequence having at least 98% identity to the sequence of SEQ ID NO:59. In some embodiments, the EPS depolymerase is encoded in a sequence having at least 99% identity to the sequence of SEQ ID NO:59.

In some embodiments, the bacteriophage shows improved host range.

In some embodiments, the bacteriophage belongs to the Genus Phikmvvirus. In some embodiments, the bacteriophage belongs to the Genus Pakpunavirus. In some embodiments, the bacteriophage belongs to the Genus Bruynoghevirus. In some embodiments, the bacteriophage belongs to the Genus Pbunavirus. In some embodiments, the bacteriophage belongs to the Genus Luzseptimavirus. In some embodiments, the bacteriophage belongs to the Genus Litunavirus. In some embodiments, the bacteriophage belongs to the Genus Nankokuvirus.

In some embodiments, the bacteriophage targets Pseudomonas. In some embodiments, the bacteriophage targets Pseudomonas aeruginosa. In some embodiments, the bacteriophage targets one or more Pseudomonas aeruginosa strains.

In some embodiments, the bacteriophage targets one or more of Pseudomonas aeruginosa, drug-resistant Pseudomonas aeruginosa, antibiotic-resistant Pseudomonas aeruginosa, and multiple antibiotic-resistant Pseudomonas aeruginosa. In some embodiments, the bacteriophage infects and kills one or more of Pseudomonas aeruginosa, antibiotic-resistant Pseudomonas aeruginosa, and multiple antibiotic-resistant Pseudomonas aeruginosa.

In some embodiments, the bacteriophage reduces biofilm mass. As used herein, the terms “reduce,” “decrease,” “reduction,” “minimal,” “low,” or “lower” refer to decreases below basal levels, e.g., as compared to a control. The terms “increase,” “high,” “higher,” “maximal,” “elevate,” or “elevation” refer to increases above basal levels, e.g., as compared to a control. Increases, elevations, decreases, or reductions can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to a control or standard level. Each of the values or ranges recited herein may include any value or subrange therebetween, including endpoints.

### Bacteriophage Compositions

In some embodiments, the present disclosure relates to one or more bacteriophage compositions comprising one or more bacteriophages engineered to express a heterologous gene as described herein (e.g., an exopolysaccharide depolymerase). In some embodiments, the target loci for insertion of the heterologous gene in bacteriophages may be chosen based on the absence of regulatory signals, genes, open reading frames (ORFs), terminators, promoters, replication origins, or any other known features that may be essential to the replication cycle of the phage. Thus, in some embodiments the heterologous gene is not inserted any such location. In some embodiments, target loci for insertion of the heterologous gene in bacteriophages may be chosen based on the expected level of expression from said locus during the phage replication cycle. In some embodiments, the target loci comprise genomic regions that are highly transcribed. In some embodiments, the heterologous gene may comprise an EPS depolymerase.

The heterologous gene can be inserted into any suitable location, for example, as described above, one that does not unduly impact an essential gene of the virus, and/or affirmatively as described above, in a location of high transcription, for example. In some embodiments, the heterologous gene may be inserted at a specific locus and may replace an entire segment from the genomic sequence of the corresponding unmodified (wild-type) phage. Alternatively or additionally, in some embodiments, the heterologous gene may be inserted at a specific locus, while the entire genomic sequence is otherwise maintained. For example, the following table provides some non-limiting examples of such insertions into various genera of bacteriophage:

TABLE 1

| | | Insertion site into |
|---|---|---|
| | | unmodified (wild-type) |
| Genus | Strain of Engineered phage | phage genomic sequence |
| Pbunavirus | APBP1-1 (SEQ ID NO: 26), | bp Δ[1173-1274] of |
| | APBP1-2 (SEQ ID NO: 27), | reference Seq ID 1 |
| | APBP1-3 (SEQ ID NO: 28), | |
| | APBP1-4 (SEQ ID NO: 29), | |
| | APBP1-5 (SEQ ID NO: 30), | |
| | APBP1-6 (SEQ ID NO: 70) | |
| Pbunavirus | APBP2-1 (SEQ ID NO: 31), | bp Δ[1163-1278] of |
| | APBP2-2 (SEQ ID NO: 32), | reference Seq ID 2 |
| | APBP2-3 (SEQ ID NO: 33), | |
| | APBP2-4 (SEQ ID NO: 34), | |
| | APBP2-5 (SEQ ID NO: 35) | |
| Pbunavirus | APBP18-1 (SEQ ID NO: 37), | bp Δ[1173-1332] of |
| | APBP18-2 (SEQ ID NO: 38), | reference Seq ID 7 |
| | APBP18-3 (SEQ ID NO: 71) | |
| Pbunavirus | APBP19-1 (SEQ ID NO: 41), | bp Δ[1173-1273] of |
| | APBP19-2 (SEQ ID NO: 42) | reference Seq ID 8 |
| Pbunavirus | APBP20-1 (SEQ ID NO: 39), | bp Δ[1174-1327] of |
| | APBP20-2 (SEQ ID NO: 40) | reference Seq ID 9 |
| Pbunavirus | APBP21-1 (SEQ ID NO: 50), | bp Δ[1173-1274] of |
| | APBP21-2 (SEQ ID NO: 51) | reference Seq ID 10 |
| Pbunavirus | APBP24-1 (SEQ ID NO: 43), | bp Δ[1177-1332] of |
| | APBP24-2 (SEQ ID NO: 44) | reference Seq ID 11 |
| Phikmvvirus | APBP3-1 (SEQ ID NO: 45), | bp 26198 of reference |
| | APBP3-2 (SEQ ID NO: 46), | Seq ID 3 |
| | APBP3-3 (SEQ ID NO: 47), | |
| | APBP3-13 (SEQ ID NO: 56), | |
| | APBP3-14 (SEQ ID NO: 57) | |
| Phikmvvirus | APBP3-5 (SEQ ID NO: 48), | bp Δ[26198-26294] of |
| | APBP3-6 (SEQ ID NO: 49), | reference Seq ID 3 |
| | APBP3-8 (SEQ ID NO: 52), | |
| | APBP3-10 (SEQ ID NO: 53), | |
| | APBP3-11 (SEQ ID NO: 54), | |
| | APBP3-12 (SEQ ID NO: 55) | |
| Phikmvvirus | APBP17-1 (SEQ ID NO: 58) | bp 43176 of reference |
| | | Seq ID 6 |
| Pakpunavirus | APBP4-1 (SEQ ID NO: 60) | bp 50549 of reference |
| | | Seq ID 4 |
| Pakpunavirus | APBP4-2 (SEQ ID NO: 61), | bp 31289 of reference |
| | APBP4-3 (SEQ ID NO: 62) | Seq ID 4 |
| Pakpunavirus | APBP4-4 (SEQ ID NO: 63) | bp 4740 of reference |
| | | Seq ID 4 |
| Pakpunavirus | APBP4-5 (SEQ ID NO: 64), | bp 26926 of reference |
| | APBP4-6 (SEQ ID NO: 65), | Seq ID 4 |
| | APBP4-8 (SEQ ID NO: 66) | |
| Bruynoghevirus | APBP6-1 (SEQ ID NO: 67), | bp Δ[2501-2540] of |
| | APBP6-2 (SEQ ID NO: 68), | reference Seq ID 5 |
| | APBP6-3 (SEQ ID NO: 69) | |

* Δ denotes a range of base pairs where the heterologous gene was inserted and the genomic region from the bacteriophage was deleted.

The above are non-limiting examples of insertions that were done. It should be understood that while a specific location or range of base pairs is listed for some of the particular strains in Table 1, that the insertion and/or deletion could also be at any base pair within the listed range and/or could remove any range of bases therein. The insertion can also be within 10 base pairs prior to or after the listed insertion point or insertion range.

The one or more bacteriophages may comprise a polynucleotide sequence selected from the polynucleotide sequence of SEQ ID NO:26, a polynucleotide sequence of SEQ ID NO:27, a polynucleotide sequence of SEQ ID NO:28, a polynucleotide sequence of SEQ ID NO:29, a polynucleotide sequence of SEQ ID NO:30, a polynucleotide sequence of SEQ ID NO:31, a polynucleotide sequence of SEQ ID NO:32, a polynucleotide sequence of SEQ ID NO:33, a polynucleotide sequence of SEQ ID NO:34, a polynucleotide sequence of SEQ ID NO:35, a polynucleotide sequence of SEQ ID NO:37, a polynucleotide sequence of SEQ ID NO:38, a polynucleotide sequence of SEQ ID NO:39, a polynucleotide sequence of SEQ ID NO:40, a polynucleotide sequence of SEQ ID NO:41, a polynucleotide sequence of SEQ ID NO:42, a polynucleotide sequence of SEQ ID NO:43, a polynucleotide sequence of SEQ ID NO:44, a polynucleotide sequence of SEQ ID NO:45, a polynucleotide sequence of SEQ ID NO:46, a polynucleotide sequence of SEQ ID NO:47, a polynucleotide sequence of SEQ ID NO:48, a polynucleotide sequence of SEQ ID NO:49, a polynucleotide sequence of SEQ ID NO:50, a polynucleotide sequence of SEQ ID NO:51, a polynucleotide sequence of SEQ ID NO:52, a polynucleotide sequence of SEQ ID NO:53, a polynucleotide sequence of SEQ ID NO:54, a polynucleotide sequence of SEQ ID NO:55, a polynucleotide sequence of SEQ ID NO:56, a polynucleotide sequence of SEQ ID NO:57, a polynucleotide sequence of SEQ ID NO:58, a polynucleotide sequence of SEQ ID NO:60, a polynucleotide sequence of SEQ ID NO:61, a polynucleotide sequence of SEQ ID NO:62, a polynucleotide sequence of SEQ ID NO:63, a polynucleotide sequence of SEQ ID NO:64, a polynucleotide sequence of SEQ ID NO:65, a polynucleotide sequence of SEQ ID NO:66, a polynucleotide sequence of SEQ ID NO:67, a polynucleotide sequence of SEQ ID NO:68, a polynucleotide sequence of SEQ ID NO:69, a polynucleotide sequence of SEQ ID NO:70, a polynucleotide sequence of SEQ ID NO:71; a polynucleotide sequence of SEQ ID NO:73; a polynucleotide sequence with at least 90% identity to SEQ ID NO:26, a polynucleotide sequence with at least 90% identity to SEQ ID NO:27, a polynucleotide sequence with at least 90% identity to SEQ ID NO:28, a polynucleotide sequence with at least 90% identity to SEQ ID NO:29, a polynucleotide sequence with at least 90% identity to SEQ ID NO:30, a polynucleotide sequence with at least 90% identity to SEQ ID NO:31, a polynucleotide sequence with at least 90% identity to SEQ ID NO:32, a polynucleotide sequence with at least 90% identity to SEQ ID NO:33, a polynucleotide sequence with at least 90% identity to SEQ ID NO:34, a polynucleotide sequence with at least 90% identity to SEQ ID NO:35, a polynucleotide sequence with at least 90% identity to SEQ ID NO:37, a polynucleotide sequence with at least 90% identity to SEQ ID NO:38, a polynucleotide sequence with at least 90% identity to SEQ ID NO:39, a polynucleotide sequence with at least 90% identity to SEQ ID NO:40, a polynucleotide sequence with at least 90% identity to SEQ ID NO:41, a polynucleotide sequence with at least 90% identity to SEQ ID NO:42, a polynucleotide sequence with at least 90% identity to SEQ ID NO:43, a polynucleotide sequence with at least 90% identity to SEQ ID NO:44, a polynucleotide sequence with at least 90% identity to SEQ ID NO:45, a polynucleotide sequence with at least 90% identity to SEQ ID NO:46, a polynucleotide sequence with at least 90% identity to SEQ ID NO:47, a polynucleotide sequence with at least 90% identity to SEQ ID NO:48, a polynucleotide sequence with at least 90% identity to SEQ ID NO:49, a polynucleotide sequence with at least 90% identity to SEQ ID NO:50, a polynucleotide sequence with at least 90% identity to SEQ ID NO:51, a polynucleotide sequence with at least 90% identity to SEQ ID NO:52, a polynucleotide sequence with at least 90% identity to SEQ ID NO:53, a polynucleotide sequence with at least 90% identity to SEQ ID NO:54, a polynucleotide sequence with at least 90% identity to SEQ ID NO:55, a polynucleotide sequence with at least 90% identity to SEQ ID NO:56, a polynucleotide sequence with at least 90% identity to SEQ ID NO:57, a polynucleotide sequence with at least 90% identity to SEQ ID NO:58, a polynucleotide sequence with at least 90% identity to SEQ ID NO:60, a polynucleotide sequence with at least 90% identity to SEQ ID NO:61, a polynucleotide sequence with at least 90% identity to SEQ ID NO:62, a polynucleotide sequence with at least 90% identity to SEQ ID NO:63, a polynucleotide sequence with at least 90% identity to SEQ ID NO:64, a polynucleotide sequence with at least 90% identity to SEQ ID NO:65, a polynucleotide sequence with at least 90% identity to SEQ ID NO:66, a polynucleotide sequence with at least 90% identity to SEQ ID NO:67, a polynucleotide sequence with at least 90% identity to SEQ ID NO:68, a polynucleotide sequence with at least 90% identity to SEQ ID NO:69, a polynucleotide sequence with at least 90% identity to SEQ ID NO:70, a polynucleotide sequence with at least 90% identity to SEQ ID NO:71, a polynucleotide sequence with at least 90% identity to SEQ ID NO:73.

In some embodiments, the one or more bacteriophages may comprise a polynucleotide sequence with at least 91% identity to SEQ ID NO:26. In some embodiments, the one or more bacteriophages may comprise a polynucleotide sequence with at least 92% identity to SEQ ID NO:26. In some embodiments, the one or more bacteriophages may comprise a polynucleotide sequence with at least 93% identity to SEQ ID NO:26. In some embodiments, the one or more bacteriophages may comprise a polynucleotide sequence with at least 94% identity to SEQ ID NO:26. In some embodiments, the one or more bacteriophages may comprise a polynucleotide sequence with at least 95% identity to SEQ ID NO:26. In some embodiments, the one or more bacteriophages may comprise a polynucleotide sequence with at least 96% identity to SEQ ID NO:26. In some embodiments, the one or more bacteriophages may comprise a polynucleotide sequence with at least 97% identity to SEQ ID NO:26. In some embodiments, the one or more bacteriophages may comprise a polynucleotide sequence with at least 98% identity to SEQ ID NO:26. In some embodiments, the one or more bacteriophages may comprise a polynucleotide sequence with at least 99% identity to SEQ ID NO:26

In some embodiments, the one or more bacteriophages may comprise a polynucleotide sequence with at least 91% identity to SEQ ID NO:27. In some embodiments, the one or more bacteriophages may comprise a polynucleotide sequence with at least 92% identity to SEQ ID NO:27. In some embodiments, the one or more bacteriophages may comprise a polynucleotide sequence with at least 93% identity to SEQ ID NO:27. In some embodiments, the one or more bacteriophages may comprise a polynucleotide sequence with at least 94% identity to SEQ ID NO:27. In some embodiments, the one or more bacteriophages may comprise a polynucleotide sequence with at least 95% identity to SEQ ID NO:27. In some embodiments, the one or more bacteriophages may comprise a polynucleotide sequence with at least 96% identity to SEQ ID NO:27. In some embodiments, the one or more bacteriophages may comprise a polynucleotide sequence with at least 97% identity to SEQ ID NO:27. In some embodiments, the one or more bacteriophages may comprise a polynucleotide sequence with at least 98% identity to SEQ ID NO:27. In some embodiments, the one or more bacteriophages may comprise a polynucleotide sequence with at least 99% identity to SEQ ID NO:27.

In some embodiments, the one or more bacteriophages may comprise a polynucleotide sequence with at least 91% identity to SEQ ID NO:28. In some embodiments, the one or more bacteriophages may comprise a polynucleotide sequence with at least 92% identity to SEQ ID NO:28. In some embodiments, the one or more bacteriophages may comprise a polynucleotide sequence with at least 93% identity to SEQ ID NO:28. In some embodiments, the one or more bacteriophages may comprise a polynucleotide sequence with at least 94% identity to SEQ ID NO:28. In some embodiments, the one or more bacteriophages may comprise a polynucleotide sequence with at least 95% identity to SEQ ID NO:28. In some embodiments, the one or more bacteriophages may comprise a polynucleotide sequence with at least 96% identity to SEQ ID NO:28. In some embodiments, the one or more bacteriophages may comprise a polynucleotide sequence with at least 97% identity to SEQ ID NO:28. In some embodiments, the one or more bacteriophages may comprise a polynucleotide sequence with at least 98% identity to SEQ ID NO:28. In some embodiments, the one or more bacteriophages may comprise a polynucleotide sequence with at least 99% identity to SEQ ID NO:28.

In some embodiments, the one or more bacteriophages may comprise a polynucleotide sequence with at least 91% identity to SEQ ID NO:29. In some embodiments, the one or more bacteriophages may comprise a polynucleotide sequence with at least 92% identity to SEQ ID NO:29. In some embodiments, the one or more bacteriophages may comprise a polynucleotide sequence with at least 93% identity to SEQ ID NO:29. In some embodiments, the one or more bacteriophages may comprise a polynucleotide sequence with at least 94% identity to SEQ ID NO:29. In some embodiments, the one or more bacteriophages may comprise a polynucleotide sequence with at least 95% identity to SEQ ID NO:29. In some embodiments, the one or more bacteriophages may comprise a polynucleotide sequence with at least 96% identity to SEQ ID NO:29. In some embodiments, the one or more bacteriophages may comprise a polynucleotide sequence with at least 97% identity to SEQ ID NO:29. In some embodiments, the one or more bacteriophages may comprise a polynucleotide sequence with at least 98% identity to SEQ ID NO:29. In some embodiments, the one or more bacteriophages may comprise a polynucleotide sequence with at least 99% identity to SEQ ID NO:29.

In some embodiments, the one or more bacteriophages may comprise a polynucleotide sequence with at least 91% identity to SEQ ID NO:30. In some embodiments, the one or more bacteriophages may comprise a polynucleotide sequence with at least 92% identity to SEQ ID NO:30. In some embodiments, the one or more bacteriophages may comprise a polynucleotide sequence with at least 93% identity to SEQ ID NO:30. In some embodiments, the one or more bacteriophages may comprise a polynucleotide sequence with at least 94% identity to SEQ ID NO:30. In some embodiments, the one or more bacteriophages may comprise a polynucleotide sequence with at least 95% identity to SEQ ID NO:30. In some embodiments, the one or more bacteriophages may comprise a polynucleotide sequence with at least 96% identity to SEQ ID NO:30. In some embodiments, the one or more bacteriophages may comprise a polynucleotide sequence with at least 97% identity to SEQ ID NO:30. In some embodiments, the one or more bacteriophages may comprise a polynucleotide sequence with at least 98% identity to SEQ ID NO:30. In some embodiments, the one or more bacteriophages may comprise a polynucleotide sequence with at least 99% identity to SEQ ID NO:30.

In some embodiments, the one or more bacteriophages may comprise a polynucleotide sequence with at least 91% identity to SEQ ID NO:31. In some embodiments, the one or more bacteriophages may comprise a polynucleotide sequence with at least 92% identity to SEQ ID NO:31. In some embodiments, the one or more bacteriophages may comprise a polynucleotide sequence with at least 93% identity to SEQ ID NO:31. In some embodiments, the one or more bacteriophages may comprise a polynucleotide sequence with at least 94% identity to SEQ ID NO:31. In some embodiments, the one or more bacteriophages may comprise a polynucleotide sequence with at least 95% identity to SEQ ID NO:31. In some embodiments, the one or more bacteriophages may comprise a polynucleotide sequence with at least 96% identity to SEQ ID NO:31. In some embodiments, the one or more bacteriophages may comprise a polynucleotide sequence with at least 97% identity to SEQ ID NO:31. In some embodiments, the one or more bacteriophages may comprise a polynucleotide sequence with at least 98% identity to SEQ ID NO:31. In some embodiments, the one or more bacteriophages may comprise a polynucleotide sequence with at least 99% identity to SEQ ID NO:31.

In some embodiments, the one or more bacteriophages may comprise a polynucleotide sequence with at least 91% identity to SEQ ID NO:32. In some embodiments, the one or more bacteriophages may comprise a polynucleotide sequence with at least 92% identity to SEQ ID NO:32. In some embodiments, the one or more bacteriophages may comprise a polynucleotide sequence with at least 93% identity to SEQ ID NO:32. In some embodiments, the one or more bacteriophages may comprise a polynucleotide sequence with at least 94% identity to SEQ ID NO:32. In some embodiments, the one or more bacteriophages may comprise a polynucleotide sequence with at least 95% identity to SEQ ID NO:32. In some embodiments, the one or more bacteriophages may comprise a polynucleotide sequence with at least 96% identity to SEQ ID NO:32. In some embodiments, the one or more bacteriophages may comprise a polynucleotide sequence with at least 97% identity to SEQ ID NO:32. In some embodiments, the one or more bacteriophages may comprise a polynucleotide sequence with at least 98% identity to SEQ ID NO:32. In some embodiments, the one or more bacteriophages may comprise a polynucleotide sequence with at least 99% identity to SEQ ID NO:32.

In some embodiments, the one or more bacteriophages may comprise a polynucleotide sequence with at least 91% identity to SEQ ID NO:33. In some embodiments, the one or more bacteriophages may comprise a polynucleotide sequence with at least 92% identity to SEQ ID NO:33. In some embodiments, the one or more bacteriophages may comprise a polynucleotide sequence with at least 93% identity to SEQ ID NO:33. In some embodiments, the one or more bacteriophages may comprise a polynucleotide sequence with at least 94% identity to SEQ ID NO:33. In some embodiments, the one or more bacteriophages may comprise a polynucleotide sequence with at least 95% identity to SEQ ID NO:33. In some embodiments, the one or more bacteriophages may comprise a polynucleotide sequence with at least 96% identity to SEQ ID NO:33. In some embodiments, the one or more bacteriophages may comprise a polynucleotide sequence with at least 97% identity to SEQ ID NO:33. In some embodiments, the one or more bacteriophages may comprise a polynucleotide sequence with at least 98% identity to SEQ ID NO:33. In some embodiments, the one or more bacteriophages may comprise a polynucleotide sequence with at least 99% identity to SEQ ID NO:33.

In some embodiments, the one or more bacteriophages may comprise a polynucleotide sequence with at least 91% identity to SEQ ID NO:34. In some embodiments, the one or more bacteriophages may comprise a polynucleotide sequence with at least 92% identity to SEQ ID NO:34. In some embodiments, the one or more bacteriophages may comprise a polynucleotide sequence with at least 93% identity to SEQ ID NO:34. In some embodiments, the one or more bacteriophages may comprise a polynucleotide sequence with at least 94% identity to SEQ ID NO:34. In some embodiments, the one or more bacteriophages may comprise a polynucleotide sequence with at least 95% identity to SEQ ID NO:34. In some embodiments, the one or more bacteriophages may comprise a polynucleotide sequence with at least 96% identity to SEQ ID NO:34. In some embodiments, the one or more bacteriophages may comprise a polynucleotide sequence with at least 97% identity to SEQ ID NO:34. In some embodiments, the one or more bacteriophages may comprise a polynucleotide sequence with at least 98% identity to SEQ ID NO:34. In some embodiments, the one or more bacteriophages may comprise a polynucleotide sequence with at least 99% identity to SEQ ID NO:34.

In some embodiments, the one or more bacteriophages may comprise a polynucleotide sequence with at least 91% identity to SEQ ID NO:35. In some embodiments, the one or more bacteriophages may comprise a polynucleotide sequence with at least 92% identity to SEQ ID NO:35. In some embodiments, the one or more bacteriophages may comprise a polynucleotide sequence with at least 93% identity to SEQ ID NO:35. In some embodiments, the one or more bacteriophages may comprise a polynucleotide sequence with at least 94% identity to SEQ ID NO:35. In some embodiments, the one or more bacteriophages may comprise a polynucleotide sequence with at least 95% identity to SEQ ID NO:35. In some embodiments, the one or more bacteriophages may comprise a polynucleotide sequence with at least 96% identity to SEQ ID NO:35. In some embodiments, the one or more bacteriophages may comprise a polynucleotide sequence with at least 97% identity to SEQ ID NO:35. In some embodiments, the one or more bacteriophages may comprise a polynucleotide sequence with at least 98% identity to SEQ ID NO:35. In some embodiments, the one or more bacteriophages may comprise a polynucleotide sequence with at least 99% identity to SEQ ID NO:35.

In some embodiments, the one or more bacteriophages may comprise a polynucleotide sequence with at least 91% identity to SEQ ID NO:37. In some embodiments, the one or more bacteriophages may comprise a polynucleotide sequence with at least 92% identity to SEQ ID NO:37. In some embodiments, the one or more bacteriophages may comprise a polynucleotide sequence with at least 93% identity to SEQ ID NO:37. In some embodiments, the one or more bacteriophages may comprise a polynucleotide sequence with at least 94% identity to SEQ ID NO:37. In some embodiments, the one or more bacteriophages may comprise a polynucleotide sequence with at least 95% identity to SEQ ID NO:37. In some embodiments, the one or more bacteriophages may comprise a polynucleotide sequence with at least 96% identity to SEQ ID NO:37. In some embodiments, the one or more bacteriophages may comprise a polynucleotide sequence with at least 97% identity to SEQ ID NO:37. In some embodiments, the one or more bacteriophages may comprise a polynucleotide sequence with at least 98% identity to SEQ ID NO:37. In some embodiments, the one or more bacteriophages may comprise a polynucleotide sequence with at least 99% identity to SEQ ID NO:37.

In some embodiments, the one or more bacteriophages may comprise a polynucleotide sequence with at least 91% identity to SEQ ID NO:38. In some embodiments, the one or more bacteriophages may comprise a polynucleotide sequence with at least 92% identity to SEQ ID NO:38. In some embodiments, the one or more bacteriophages may comprise a polynucleotide sequence with at least 93% identity to SEQ ID NO:38. In some embodiments, the one or more bacteriophages may comprise a polynucleotide sequence with at least 94% identity to SEQ ID NO:38. In some embodiments, the one or more bacteriophages may comprise a polynucleotide sequence with at least 95% identity to SEQ ID NO:38. In some embodiments, the one or more bacteriophages may comprise a polynucleotide sequence with at least 96% identity to SEQ ID NO:38. In some embodiments, the one or more bacteriophages may comprise a polynucleotide sequence with at least 97% identity to SEQ ID NO:38. In some embodiments, the one or more bacteriophages may comprise a polynucleotide sequence with at least 98% identity to SEQ ID NO:38. In some embodiments, the one or more bacteriophages may comprise a polynucleotide sequence with at least 99% identity to SEQ ID NO:38.

In some embodiments, the one or more bacteriophages may comprise a polynucleotide sequence with at least 91% identity to SEQ ID NO:39. In some embodiments, the one or more bacteriophages may comprise a polynucleotide sequence with at least 92% identity to SEQ ID NO:39. In some embodiments, the one or more bacteriophages may comprise a polynucleotide sequence with at least 93% identity to SEQ ID NO:39. In some embodiments, the one or more bacteriophages may comprise a polynucleotide sequence with at least 94% identity to SEQ ID NO:39. In some embodiments, the one or more bacteriophages may comprise a polynucleotide sequence with at least 95% identity to SEQ ID NO:39. In some embodiments, the one or more bacteriophages may comprise a polynucleotide sequence with at least 96% identity to SEQ ID NO:39. In some embodiments, the one or more bacteriophages may comprise a polynucleotide sequence with at least 97% identity to SEQ ID NO:39. In some embodiments, the one or more bacteriophages may comprise a polynucleotide sequence with at least 98% identity to SEQ ID NO:39. In some embodiments, the one or more bacteriophages may comprise a polynucleotide sequence with at least 99% identity to SEQ ID NO:39.

In some embodiments, the one or more bacteriophages may comprise a polynucleotide sequence with at least 91% identity to SEQ ID NO:40. In some embodiments, the one or more bacteriophages may comprise a polynucleotide sequence with at least 92% identity to SEQ ID NO:40. In some embodiments, the one or more bacteriophages may comprise a polynucleotide sequence with at least 93% identity to SEQ ID NO:40. In some embodiments, the one or more bacteriophages may comprise a polynucleotide sequence with at least 94% identity to SEQ ID NO:40. In some embodiments, the one or more bacteriophages may comprise a polynucleotide sequence with at least 95% identity to SEQ ID NO:40. In some embodiments, the one or more bacteriophages may comprise a polynucleotide sequence with at least 96% identity to SEQ ID NO:40. In some embodiments, the one or more bacteriophages may comprise a polynucleotide sequence with at least 97% identity to SEQ ID NO:40. In some embodiments, the one or more bacteriophages may comprise a polynucleotide sequence with at least 98% identity to SEQ ID NO:40. In some embodiments, the one or more bacteriophages may comprise a polynucleotide sequence with at least 99% identity to SEQ ID NO:40.

In some embodiments, the one or more bacteriophages may comprise a polynucleotide sequence with at least 91% identity to SEQ ID NO:41. In some embodiments, the one or more bacteriophages may comprise a polynucleotide sequence with at least 92% identity to SEQ ID NO:41. In some embodiments, the one or more bacteriophages may comprise a polynucleotide sequence with at least 93% identity to SEQ ID NO:41. In some embodiments, the one or more bacteriophages may comprise a polynucleotide sequence with at least 94% identity to SEQ ID NO:41. In some embodiments, the one or more bacteriophages may comprise a polynucleotide sequence with at least 95% identity to SEQ ID NO:41. In some embodiments, the one or more bacteriophages may comprise a polynucleotide sequence with at least 96% identity to SEQ ID NO:41. In some embodiments, the one or more bacteriophages may comprise a polynucleotide sequence with at least 97% identity to SEQ ID NO:41. In some embodiments, the one or more bacteriophages may comprise a polynucleotide sequence with at least 98% identity to SEQ ID NO:41. In some embodiments, the one or more bacteriophages may comprise a polynucleotide sequence with at least 99% identity to SEQ ID NO:41.

In some embodiments, the one or more bacteriophages may comprise a polynucleotide sequence with at least 91% identity to SEQ ID NO:42. In some embodiments, the one or more bacteriophages may comprise a polynucleotide sequence with at least 92% identity to SEQ ID NO:42. In some embodiments, the one or more bacteriophages may comprise a polynucleotide sequence with at least 93% identity to SEQ ID NO:42. In some embodiments, the one or more bacteriophages may comprise a polynucleotide sequence with at least 94% identity to SEQ ID NO:42. In some embodiments, the one or more bacteriophages may comprise a polynucleotide sequence with at least 95% identity to SEQ ID NO:42. In some embodiments, the one or more bacteriophages may comprise a polynucleotide sequence with at least 96% identity to SEQ ID NO:42. In some embodiments, the one or more bacteriophages may comprise a polynucleotide sequence with at least 97% identity to SEQ ID NO:42. In some embodiments, the one or more bacteriophages may comprise a polynucleotide sequence with at least 98% identity to SEQ ID NO:42. In some embodiments, the one or more bacteriophages may comprise a polynucleotide sequence with at least 99% identity to SEQ ID NO:42.

In some embodiments, the one or more bacteriophages may comprise a polynucleotide sequence with at least 91% identity to SEQ ID NO:43. In some embodiments, the one or more bacteriophages may comprise a polynucleotide sequence with at least 92% identity to SEQ ID NO:43. In some embodiments, the one or more bacteriophages may comprise a polynucleotide sequence with at least 93% identity to SEQ ID NO:43. In some embodiments, the one or more bacteriophages may comprise a polynucleotide sequence with at least 94% identity to SEQ ID NO:43. In some embodiments, the one or more bacteriophages may comprise a polynucleotide sequence with at least 95% identity to SEQ ID NO:43. In some embodiments, the one or more bacteriophages may comprise a polynucleotide sequence with at least 96% identity to SEQ ID NO:43. In some embodiments, the one or more bacteriophages may comprise a polynucleotide sequence with at least 97% identity to SEQ ID NO:43. In some embodiments, the one or more bacteriophages may comprise a polynucleotide sequence with at least 98% identity to SEQ ID NO:43. In some embodiments, the one or more bacteriophages may comprise a polynucleotide sequence with at least 99% identity to SEQ ID NO:43.

In some embodiments, the one or more bacteriophages may comprise a polynucleotide sequence with at least 91% identity to SEQ ID NO:44. In some embodiments, the one or more bacteriophages may comprise a polynucleotide sequence with at least 92% identity to SEQ ID NO:44. In some embodiments, the one or more bacteriophages may comprise a polynucleotide sequence with at least 93% identity to SEQ ID NO:44. In some embodiments, the one or more bacteriophages may comprise a polynucleotide sequence with at least 94% identity to SEQ ID NO:44. In some embodiments, the one or more bacteriophages may comprise a polynucleotide sequence with at least 95% identity to SEQ ID NO:44. In some embodiments, the one or more bacteriophages may comprise a polynucleotide sequence with at least 96% identity to SEQ ID NO:44. In some embodiments, the one or more bacteriophages may comprise a polynucleotide sequence with at least 97% identity to SEQ ID NO:44. In some embodiments, the one or more bacteriophages may comprise a polynucleotide sequence with at least 98% identity to SEQ ID NO:44. In some embodiments, the one or more bacteriophages may comprise a polynucleotide sequence with at least 99% identity to SEQ ID NO:44.

In some embodiments, the one or more bacteriophages may comprise a polynucleotide sequence with at least 91% identity to SEQ ID NO:45. In some embodiments, the one or more bacteriophages may comprise a polynucleotide sequence with at least 92% identity to SEQ ID NO:45. In some embodiments, the one or more bacteriophages may comprise a polynucleotide sequence with at least 93% identity to SEQ ID NO:45. In some embodiments, the one or more bacteriophages may comprise a polynucleotide sequence with at least 94% identity to SEQ ID NO:45. In some embodiments, the one or more bacteriophages may comprise a polynucleotide sequence with at least 95% identity to SEQ ID NO:45. In some embodiments, the one or more bacteriophages may comprise a polynucleotide sequence with at least 96% identity to SEQ ID NO:45. In some embodiments, the one or more bacteriophages may comprise a polynucleotide sequence with at least 97% identity to SEQ ID NO:45. In some embodiments, the one or more bacteriophages may comprise a polynucleotide sequence with at least 98% identity to SEQ ID NO:45. In some embodiments, the one or more bacteriophages may comprise a polynucleotide sequence with at least 99% identity to SEQ ID NO:45.

In some embodiments, the one or more bacteriophages may comprise a polynucleotide sequence with at least 91% identity to SEQ ID NO:46. In some embodiments, the one or more bacteriophages may comprise a polynucleotide sequence with at least 92% identity to SEQ ID NO:46. In some embodiments, the one or more bacteriophages may comprise a polynucleotide sequence with at least 93% identity to SEQ ID NO:46. In some embodiments, the one or more bacteriophages may comprise a polynucleotide sequence with at least 94% identity to SEQ ID NO:46. In some embodiments, the one or more bacteriophages may comprise a polynucleotide sequence with at least 95% identity to SEQ ID NO:46. In some embodiments, the one or more bacteriophages may comprise a polynucleotide sequence with at least 96% identity to SEQ ID NO:46. In some embodiments, the one or more bacteriophages may comprise a polynucleotide sequence with at least 97% identity to SEQ ID NO:46. In some embodiments, the one or more bacteriophages may comprise a polynucleotide sequence with at least 98% identity to SEQ ID NO:46. In some embodiments, the one or more bacteriophages may comprise a polynucleotide sequence with at least 99% identity to SEQ ID NO:46.

In some embodiments, the one or more bacteriophages may comprise a polynucleotide sequence with at least 91% identity to SEQ ID NO:47. In some embodiments, the one or more bacteriophages may comprise a polynucleotide sequence with at least 92% identity to SEQ ID NO:47. In some embodiments, the one or more bacteriophages may comprise a polynucleotide sequence with at least 93% identity to SEQ ID NO:47. In some embodiments, the one or more bacteriophages may comprise a polynucleotide sequence with at least 94% identity to SEQ ID NO:47. In some embodiments, the one or more bacteriophages may comprise a polynucleotide sequence with at least 95% identity to SEQ ID NO:47. In some embodiments, the one or more bacteriophages may comprise a polynucleotide sequence with at least 96% identity to SEQ ID NO:47. In some embodiments, the one or more bacteriophages may comprise a polynucleotide sequence with at least 97% identity to SEQ ID NO:47. In some embodiments, the one or more bacteriophages may comprise a polynucleotide sequence with at least 98% identity to SEQ ID NO:47. In some embodiments, the one or more bacteriophages may comprise a polynucleotide sequence with at least 99% identity to SEQ ID NO:47.

In some embodiments, the one or more bacteriophages may comprise a polynucleotide sequence with at least 91% identity to SEQ ID NO:48. In some embodiments, the one or more bacteriophages may comprise a polynucleotide sequence with at least 92% identity to SEQ ID NO:48. In some embodiments, the one or more bacteriophages may comprise a polynucleotide sequence with at least 93% identity to SEQ ID NO:48. In some embodiments, the one or more bacteriophages may comprise a polynucleotide sequence with at least 94% identity to SEQ ID NO:48. In some embodiments, the one or more bacteriophages may comprise a polynucleotide sequence with at least 95% identity to SEQ ID NO:48. In some embodiments, the one or more bacteriophages may comprise a polynucleotide sequence with at least 96% identity to SEQ ID NO:48. In some embodiments, the one or more bacteriophages may comprise a polynucleotide sequence with at least 97% identity to SEQ ID NO:48. In some embodiments, the one or more bacteriophages may comprise a polynucleotide sequence with at least 98% identity to SEQ ID NO:48. In some embodiments, the one or more bacteriophages may comprise a polynucleotide sequence with at least 99% identity to SEQ ID NO:48.

In some embodiments, the one or more bacteriophages may comprise a polynucleotide sequence with at least 91% identity to SEQ ID NO:49. In some embodiments, the one or more bacteriophages may comprise a polynucleotide sequence with at least 92% identity to SEQ ID NO:49. In some embodiments, the one or more bacteriophages may comprise a polynucleotide sequence with at least 93% identity to SEQ ID NO:49. In some embodiments, the one or more bacteriophages may comprise a polynucleotide sequence with at least 94% identity to SEQ ID NO:49. In some embodiments, the one or more bacteriophages may comprise a polynucleotide sequence with at least 95% identity to SEQ ID NO:49. In some embodiments, the one or more bacteriophages may comprise a polynucleotide sequence with at least 96% identity to SEQ ID NO:49. In some embodiments, the one or more bacteriophages may comprise a polynucleotide sequence with at least 97% identity to SEQ ID NO:49. In some embodiments, the one or more bacteriophages may comprise a polynucleotide sequence with at least 98% identity to SEQ ID NO:49. In some embodiments, the one or more bacteriophages may comprise a polynucleotide sequence with at least 99% identity to SEQ ID NO:49.

In some embodiments, the one or more bacteriophages may comprise a polynucleotide sequence with at least 91% identity to SEQ ID NO:50. In some embodiments, the one or more bacteriophages may comprise a polynucleotide sequence with at least 92% identity to SEQ ID NO:50. In some embodiments, the one or more bacteriophages may comprise a polynucleotide sequence with at least 93% identity to SEQ ID NO:50. In some embodiments, the one or more bacteriophages may comprise a polynucleotide sequence with at least 94% identity to SEQ ID NO:50. In some embodiments, the one or more bacteriophages may comprise a polynucleotide sequence with at least 95% identity to SEQ ID NO:50. In some embodiments, the one or more bacteriophages may comprise a polynucleotide sequence with at least 96% identity to SEQ ID NO:50. In some embodiments, the one or more bacteriophages may comprise a polynucleotide sequence with at least 97% identity to SEQ ID NO:50. In some embodiments, the one or more bacteriophages may comprise a polynucleotide sequence with at least 98% identity to SEQ ID NO:50. In some embodiments, the one or more bacteriophages may comprise a polynucleotide sequence with at least 99% identity to SEQ ID NO:50.

In some embodiments, the one or more bacteriophages may comprise a polynucleotide sequence with at least 91% identity to SEQ ID NO:51. In some embodiments, the one or more bacteriophages may comprise a polynucleotide sequence with at least 92% identity to SEQ ID NO:51. In some embodiments, the one or more bacteriophages may comprise a polynucleotide sequence with at least 93% identity to SEQ ID NO:51. In some embodiments, the one or more bacteriophages may comprise a polynucleotide sequence with at least 94% identity to SEQ ID NO:51. In some embodiments, the one or more bacteriophages may comprise a polynucleotide sequence with at least 95% identity to SEQ ID NO:51. In some embodiments, the one or more bacteriophages may comprise a polynucleotide sequence with at least 96% identity to SEQ ID NO:51. In some embodiments, the one or more bacteriophages may comprise a polynucleotide sequence with at least 97% identity to SEQ ID NO:51. In some embodiments, the one or more bacteriophages may comprise a polynucleotide sequence with at least 98% identity to SEQ ID NO:51. In some embodiments, the one or more bacteriophages may comprise a polynucleotide sequence with at least 99% identity to SEQ ID NO:51.

In some embodiments, the one or more bacteriophages may comprise a polynucleotide sequence with at least 91% identity to SEQ ID NO:52. In some embodiments, the one or more bacteriophages may comprise a polynucleotide sequence with at least 92% identity to SEQ ID NO:52. In some embodiments, the one or more bacteriophages may comprise a polynucleotide sequence with at least 93% identity to SEQ ID NO:52. In some embodiments, the one or more bacteriophages may comprise a polynucleotide sequence with at least 94% identity to SEQ ID NO:52. In some embodiments, the one or more bacteriophages may comprise a polynucleotide sequence with at least 95% identity to SEQ ID NO:52. In some embodiments, the one or more bacteriophages may comprise a polynucleotide sequence with at least 96% identity to SEQ ID NO:52. In some embodiments, the one or more bacteriophages may comprise a polynucleotide sequence with at least 97% identity to SEQ ID NO:52. In some embodiments, the one or more bacteriophages may comprise a polynucleotide sequence with at least 98% identity to SEQ ID NO:52. In some embodiments, the one or more bacteriophages may comprise a polynucleotide sequence with at least 99% identity to SEQ ID NO:52.

In some embodiments, the one or more bacteriophages may comprise a polynucleotide sequence with at least 91% identity to SEQ ID NO:53. In some embodiments, the one or more bacteriophages may comprise a polynucleotide sequence with at least 92% identity to SEQ ID NO:53. In some embodiments, the one or more bacteriophages may comprise a polynucleotide sequence with at least 93% identity to SEQ ID NO:53. In some embodiments, the one or more bacteriophages may comprise a polynucleotide sequence with at least 94% identity to SEQ ID NO:53. In some embodiments, the one or more bacteriophages may comprise a polynucleotide sequence with at least 95% identity to SEQ ID NO:53. In some embodiments, the one or more bacteriophages may comprise a polynucleotide sequence with at least 96% identity to SEQ ID NO:53. In some embodiments, the one or more bacteriophages may comprise a polynucleotide sequence with at least 97% identity to SEQ ID NO:53. In some embodiments, the one or more bacteriophages may comprise a polynucleotide sequence with at least 98% identity to SEQ ID NO:53. In some embodiments, the one or more bacteriophages may comprise a polynucleotide sequence with at least 99% identity to SEQ ID NO:53.

In some embodiments, the one or more bacteriophages may comprise a polynucleotide sequence with at least 91% identity to SEQ ID NO:54. In some embodiments, the one or more bacteriophages may comprise a polynucleotide sequence with at least 92% identity to SEQ ID NO:54. In some embodiments, the one or more bacteriophages may comprise a polynucleotide sequence with at least 93% identity to SEQ ID NO:54. In some embodiments, the one or more bacteriophages may comprise a polynucleotide sequence with at least 94% identity to SEQ ID NO:54. In some embodiments, the one or more bacteriophages may comprise a polynucleotide sequence with at least 95% identity to SEQ ID NO:54. In some embodiments, the one or more bacteriophages may comprise a polynucleotide sequence with at least 96% identity to SEQ ID NO:54. In some embodiments, the one or more bacteriophages may comprise a polynucleotide sequence with at least 97% identity to SEQ ID NO:54. In some embodiments, the one or more bacteriophages may comprise a polynucleotide sequence with at least 98% identity to SEQ ID NO:54. In some embodiments, the one or more bacteriophages may comprise a polynucleotide sequence with at least 99% identity to SEQ ID NO:54.

In some embodiments, the one or more bacteriophages may comprise a polynucleotide sequence with at least 91% identity to SEQ ID NO:55. In some embodiments, the one or more bacteriophages may comprise a polynucleotide sequence with at least 92% identity to SEQ ID NO:55. In some embodiments, the one or more bacteriophages may comprise a polynucleotide sequence with at least 93% identity to SEQ ID NO:55. In some embodiments, the one or more bacteriophages may comprise a polynucleotide sequence with at least 94% identity to SEQ ID NO:55. In some embodiments, the one or more bacteriophages may comprise a polynucleotide sequence with at least 95% identity to SEQ ID NO:55. In some embodiments, the one or more bacteriophages may comprise a polynucleotide sequence with at least 96% identity to SEQ ID NO:55. In some embodiments, the one or more bacteriophages may comprise a polynucleotide sequence with at least 97% identity to SEQ ID NO:55. In some embodiments, the one or more bacteriophages may comprise a polynucleotide sequence with at least 98% identity to SEQ ID NO:55. In some embodiments, the one or more bacteriophages may comprise a polynucleotide sequence with at least 99% identity to SEQ ID NO:55.

In some embodiments, the one or more bacteriophages may comprise a polynucleotide sequence with at least 91% identity to SEQ ID NO:56. In some embodiments, the one or more bacteriophages may comprise a polynucleotide sequence with at least 92% identity to SEQ ID NO:56. In some embodiments, the one or more bacteriophages may comprise a polynucleotide sequence with at least 93% identity to SEQ ID NO:56. In some embodiments, the one or more bacteriophages may comprise a polynucleotide sequence with at least 94% identity to SEQ ID NO:56. In some embodiments, the one or more bacteriophages may comprise a polynucleotide sequence with at least 95% identity to SEQ ID NO:56. In some embodiments, the one or more bacteriophages may comprise a polynucleotide sequence with at least 96% identity to SEQ ID NO:56. In some embodiments, the one or more bacteriophages may comprise a polynucleotide sequence with at least 97% identity to SEQ ID NO:56. In some embodiments, the one or more bacteriophages may comprise a polynucleotide sequence with at least 98% identity to SEQ ID NO:56. In some embodiments, the one or more bacteriophages may comprise a polynucleotide sequence with at least 99% identity to SEQ ID NO:56.

In some embodiments, the one or more bacteriophages may comprise a polynucleotide sequence with at least 91% identity to SEQ ID NO:57. In some embodiments, the one or more bacteriophages may comprise a polynucleotide sequence with at least 92% identity to SEQ ID NO:57. In some embodiments, the one or more bacteriophages may comprise a polynucleotide sequence with at least 93% identity to SEQ ID NO:57. In some embodiments, the one or more bacteriophages may comprise a polynucleotide sequence with at least 94% identity to SEQ ID NO:57. In some embodiments, the one or more bacteriophages may comprise a polynucleotide sequence with at least 95% identity to SEQ ID NO:57. In some embodiments, the one or more bacteriophages may comprise a polynucleotide sequence with at least 96% identity to SEQ ID NO:57. In some embodiments, the one or more bacteriophages may comprise a polynucleotide sequence with at least 97% identity to SEQ ID NO:57. In some embodiments, the one or more bacteriophages may comprise a polynucleotide sequence with at least 98% identity to SEQ ID NO:57. In some embodiments, the one or more bacteriophages may comprise a polynucleotide sequence with at least 99% identity to SEQ ID NO:57.

In some embodiments, the one or more bacteriophages may comprise a polynucleotide sequence with at least 91% identity to SEQ ID NO:58. In some embodiments, the one or more bacteriophages may comprise a polynucleotide sequence with at least 92% identity to SEQ ID NO:58. In some embodiments, the one or more bacteriophages may comprise a polynucleotide sequence with at least 93% identity to SEQ ID NO:58. In some embodiments, the one or more bacteriophages may comprise a polynucleotide sequence with at least 94% identity to SEQ ID NO:58. In some embodiments, the one or more bacteriophages may comprise a polynucleotide sequence with at least 95% identity to SEQ ID NO:58. In some embodiments, the one or more bacteriophages may comprise a polynucleotide sequence with at least 96% identity to SEQ ID NO:58. In some embodiments, the one or more bacteriophages may comprise a polynucleotide sequence with at least 97% identity to SEQ ID NO:58. In some embodiments, the one or more bacteriophages may comprise a polynucleotide sequence with at least 98% identity to SEQ ID NO:58. In some embodiments, the one or more bacteriophages may comprise a polynucleotide sequence with at least 99% identity to SEQ ID NO:58.

In some embodiments, the one or more bacteriophages may comprise a polynucleotide sequence with at least 91% identity to SEQ ID NO:60. In some embodiments, the one or more bacteriophages may comprise a polynucleotide sequence with at least 92% identity to SEQ ID NO:60. In some embodiments, the one or more bacteriophages may comprise a polynucleotide sequence with at least 93% identity to SEQ ID NO:60. In some embodiments, the one or more bacteriophages may comprise a polynucleotide sequence with at least 94% identity to SEQ ID NO:60. In some embodiments, the one or more bacteriophages may comprise a polynucleotide sequence with at least 95% identity to SEQ ID NO:60. In some embodiments, the one or more bacteriophages may comprise a polynucleotide sequence with at least 96% identity to SEQ ID NO:60. In some embodiments, the one or more bacteriophages may comprise a polynucleotide sequence with at least 97% identity to SEQ ID NO:60. In some embodiments, the one or more bacteriophages may comprise a polynucleotide sequence with at least 98% identity to SEQ ID NO:60. In some embodiments, the one or more bacteriophages may comprise a polynucleotide sequence with at least 99% identity to SEQ ID NO:60.

In some embodiments, the one or more bacteriophages may comprise a polynucleotide sequence with at least 91% identity to SEQ ID NO:61. In some embodiments, the one or more bacteriophages may comprise a polynucleotide sequence with at least 92% identity to SEQ ID NO:61. In some embodiments, the one or more bacteriophages may comprise a polynucleotide sequence with at least 93% identity to SEQ ID NO:61. In some embodiments, the one or more bacteriophages may comprise a polynucleotide sequence with at least 94% identity to SEQ ID NO:61. In some embodiments, the one or more bacteriophages may comprise a polynucleotide sequence with at least 95% identity to SEQ ID NO:61. In some embodiments, the one or more bacteriophages may comprise a polynucleotide sequence with at least 96% identity to SEQ ID NO:61. In some embodiments, the one or more bacteriophages may comprise a polynucleotide sequence with at least 97% identity to SEQ ID NO:61. In some embodiments, the one or more bacteriophages may comprise a polynucleotide sequence with at least 98% identity to SEQ ID NO:61. In some embodiments, the one or more bacteriophages may comprise a polynucleotide sequence with at least 99% identity to SEQ ID NO:61.

In some embodiments, the one or more bacteriophages may comprise a polynucleotide sequence with at least 91% identity to SEQ ID NO:62. In some embodiments, the one or more bacteriophages may comprise a polynucleotide sequence with at least 92% identity to SEQ ID NO:62. In some embodiments, the one or more bacteriophages may comprise a polynucleotide sequence with at least 93% identity to SEQ ID NO:62. In some embodiments, the one or more bacteriophages may comprise a polynucleotide sequence with at least 94% identity to SEQ ID NO:62. In some embodiments, the one or more bacteriophages may comprise a polynucleotide sequence with at least 95% identity to SEQ ID NO:62. In some embodiments, the one or more bacteriophages may comprise a polynucleotide sequence with at least 96% identity to SEQ ID NO:62. In some embodiments, the one or more bacteriophages may comprise a polynucleotide sequence with at least 97% identity to SEQ ID NO:62. In some embodiments, the one or more bacteriophages may comprise a polynucleotide sequence with at least 98% identity to SEQ ID NO:62. In some embodiments, the one or more bacteriophages may comprise a polynucleotide sequence with at least 99% identity to SEQ ID NO:62.

In some embodiments, the one or more bacteriophages may comprise a polynucleotide sequence with at least 91% identity to SEQ ID NO:63. In some embodiments, the one or more bacteriophages may comprise a polynucleotide sequence with at least 92% identity to SEQ ID NO:63. In some embodiments, the one or more bacteriophages may comprise a polynucleotide sequence with at least 93% identity to SEQ ID NO:63. In some embodiments, the one or more bacteriophages may comprise a polynucleotide sequence with at least 94% identity to SEQ ID NO:63. In some embodiments, the one or more bacteriophages may comprise a polynucleotide sequence with at least 95% identity to SEQ ID NO:63. In some embodiments, the one or more bacteriophages may comprise a polynucleotide sequence with at least 96% identity to SEQ ID NO:63. In some embodiments, the one or more bacteriophages may comprise a polynucleotide sequence with at least 97% identity to SEQ ID NO:63. In some embodiments, the one or more bacteriophages may comprise a polynucleotide sequence with at least 98% identity to SEQ ID NO:63. In some embodiments, the one or more bacteriophages may comprise a polynucleotide sequence with at least 99% identity to SEQ ID NO:63.

In some embodiments, the one or more bacteriophages may comprise a polynucleotide sequence with at least 91% identity to SEQ ID NO:64. In some embodiments, the one or more bacteriophages may comprise a polynucleotide sequence with at least 92% identity to SEQ ID NO:64. In some embodiments, the one or more bacteriophages may comprise a polynucleotide sequence with at least 93% identity to SEQ ID NO:64. In some embodiments, the one or more bacteriophages may comprise a polynucleotide sequence with at least 94% identity to SEQ ID NO:64. In some embodiments, the one or more bacteriophages may comprise a polynucleotide sequence with at least 95% identity to SEQ ID NO:64. In some embodiments, the one or more bacteriophages may comprise a polynucleotide sequence with at least 96% identity to SEQ ID NO:64. In some embodiments, the one or more bacteriophages may comprise a polynucleotide sequence with at least 97% identity to SEQ ID NO:64. In some embodiments, the one or more bacteriophages may comprise a polynucleotide sequence with at least 98% identity to SEQ ID NO:64. In some embodiments, the one or more bacteriophages may comprise a polynucleotide sequence with at least 99% identity to SEQ ID NO:64.

In some embodiments, the one or more bacteriophages may comprise a polynucleotide sequence with at least 91% identity to SEQ ID NO:65. In some embodiments, the one or more bacteriophages may comprise a polynucleotide sequence with at least 92% identity to SEQ ID NO:65. In some embodiments, the one or more bacteriophages may comprise a polynucleotide sequence with at least 93% identity to SEQ ID NO:65. In some embodiments, the one or more bacteriophages may comprise a polynucleotide sequence with at least 94% identity to SEQ ID NO:65. In some embodiments, the one or more bacteriophages may comprise a polynucleotide sequence with at least 95% identity to SEQ ID NO:65. In some embodiments, the one or more bacteriophages may comprise a polynucleotide sequence with at least 96% identity to SEQ ID NO:65. In some embodiments, the one or more bacteriophages may comprise a polynucleotide sequence with at least 97% identity to SEQ ID NO:65. In some embodiments, the one or more bacteriophages may comprise a polynucleotide sequence with at least 98% identity to SEQ ID NO:65. In some embodiments, the one or more bacteriophages may comprise a polynucleotide sequence with at least 99% identity to SEQ ID NO:65.

In some embodiments, the one or more bacteriophages may comprise a polynucleotide sequence with at least 91% identity to SEQ ID NO:66. In some embodiments, the one or more bacteriophages may comprise a polynucleotide sequence with at least 92% identity to SEQ ID NO:66. In some embodiments, the one or more bacteriophages may comprise a polynucleotide sequence with at least 93% identity to SEQ ID NO:66. In some embodiments, the one or more bacteriophages may comprise a polynucleotide sequence with at least 94% identity to SEQ ID NO:66. In some embodiments, the one or more bacteriophages may comprise a polynucleotide sequence with at least 95% identity to SEQ ID NO:66. In some embodiments, the one or more bacteriophages may comprise a polynucleotide sequence with at least 96% identity to SEQ ID NO:66. In some embodiments, the one or more bacteriophages may comprise a polynucleotide sequence with at least 97% identity to SEQ ID NO:66. In some embodiments, the one or more bacteriophages may comprise a polynucleotide sequence with at least 98% identity to SEQ ID NO:66. In some embodiments, the one or more bacteriophages may comprise a polynucleotide sequence with at least 99% identity to SEQ ID NO:66.

In some embodiments, the one or more bacteriophages may comprise a polynucleotide sequence with at least 91% identity to SEQ ID NO:67. In some embodiments, the one or more bacteriophages may comprise a polynucleotide sequence with at least 92% identity to SEQ ID NO:67. In some embodiments, the one or more bacteriophages may comprise a polynucleotide sequence with at least 93% identity to SEQ ID NO:67. In some embodiments, the one or more bacteriophages may comprise a polynucleotide sequence with at least 94% identity to SEQ ID NO:67. In some embodiments, the one or more bacteriophages may comprise a polynucleotide sequence with at least 95% identity to SEQ ID NO:67. In some embodiments, the one or more bacteriophages may comprise a polynucleotide sequence with at least 96% identity to SEQ ID NO:67. In some embodiments, the one or more bacteriophages may comprise a polynucleotide sequence with at least 97% identity to SEQ ID NO:67. In some embodiments, the one or more bacteriophages may comprise a polynucleotide sequence with at least 98% identity to SEQ ID NO:67. In some embodiments, the one or more bacteriophages may comprise a polynucleotide sequence with at least 99% identity to SEQ ID NO:67.

In some embodiments, the one or more bacteriophages may comprise a polynucleotide sequence with at least 91% identity to SEQ ID NO:68. In some embodiments, the one or more bacteriophages may comprise a polynucleotide sequence with at least 92% identity to SEQ ID NO:68. In some embodiments, the one or more bacteriophages may comprise a polynucleotide sequence with at least 93% identity to SEQ ID NO:68. In some embodiments, the one or more bacteriophages may comprise a polynucleotide sequence with at least 94% identity to SEQ ID NO:68. In some embodiments, the one or more bacteriophages may comprise a polynucleotide sequence with at least 95% identity to SEQ ID NO:68. In some embodiments, the one or more bacteriophages may comprise a polynucleotide sequence with at least 96% identity to SEQ ID NO:68. In some embodiments, the one or more bacteriophages may comprise a polynucleotide sequence with at least 97% identity to SEQ ID NO:68. In some embodiments, the one or more bacteriophages may comprise a polynucleotide sequence with at least 98% identity to SEQ ID NO:68. In some embodiments, the one or more bacteriophages may comprise a polynucleotide sequence with at least 99% identity to SEQ ID NO:68.

In some embodiments, the one or more bacteriophages may comprise a polynucleotide sequence with at least 91% identity to SEQ ID NO:69. In some embodiments, the one or more bacteriophages may comprise a polynucleotide sequence with at least 92% identity to SEQ ID NO:69. In some embodiments, the one or more bacteriophages may comprise a polynucleotide sequence with at least 93% identity to SEQ ID NO:69. In some embodiments, the one or more bacteriophages may comprise a polynucleotide sequence with at least 94% identity to SEQ ID NO:69. In some embodiments, the one or more bacteriophages may comprise a polynucleotide sequence with at least 95% identity to SEQ ID NO:69. In some embodiments, the one or more bacteriophages may comprise a polynucleotide sequence with at least 96% identity to SEQ ID NO:69. In some embodiments, the one or more bacteriophages may comprise a polynucleotide sequence with at least 97% identity to SEQ ID NO:69. In some embodiments, the one or more bacteriophages may comprise a polynucleotide sequence with at least 98% identity to SEQ ID NO:69. In some embodiments, the one or more bacteriophages may comprise a polynucleotide sequence with at least 99% identity to SEQ ID NO:69.

In some embodiments, the one or more bacteriophages may comprise a polynucleotide sequence with at least 91% identity to SEQ ID NO:70. In some embodiments, the one or more bacteriophages may comprise a polynucleotide sequence with at least 92% identity to SEQ ID NO:70. In some embodiments, the one or more bacteriophages may comprise a polynucleotide sequence with at least 93% identity to SEQ ID NO:70. In some embodiments, the one or more bacteriophages may comprise a polynucleotide sequence with at least 94% identity to SEQ ID NO:70. In some embodiments, the one or more bacteriophages may comprise a polynucleotide sequence with at least 95% identity to SEQ ID NO:70. In some embodiments, the one or more bacteriophages may comprise a polynucleotide sequence with at least 96% identity to SEQ ID NO:70. In some embodiments, the one or more bacteriophages may comprise a polynucleotide sequence with at least 97% identity to SEQ ID NO:70. In some embodiments, the one or more bacteriophages may comprise a polynucleotide sequence with at least 98% identity to SEQ ID NO:70. In some embodiments, the one or more bacteriophages may comprise a polynucleotide sequence with at least 99% identity to SEQ ID NO:70.

In some embodiments, the one or more bacteriophages may comprise a polynucleotide sequence with at least 91% identity to SEQ ID NO:71. In some embodiments, the one or more bacteriophages may comprise a polynucleotide sequence with at least 92% identity to SEQ ID NO:71. In some embodiments, the one or more bacteriophages may comprise a polynucleotide sequence with at least 93% identity to SEQ ID NO:71. In some embodiments, the one or more bacteriophages may comprise a polynucleotide sequence with at least 94% identity to SEQ ID NO:71. In some embodiments, the one or more bacteriophages may comprise a polynucleotide sequence with at least 95% identity to SEQ ID NO:71. In some embodiments, the one or more bacteriophages may comprise a polynucleotide sequence with at least 96% identity to SEQ ID NO:71. In some embodiments, the one or more bacteriophages may comprise a polynucleotide sequence with at least 97% identity to SEQ ID NO:71. In some embodiments, the one or more bacteriophages may comprise a polynucleotide sequence with at least 98% identity to SEQ ID NO:71. In some embodiments, the one or more bacteriophages may comprise a polynucleotide sequence with at least 99% identity to SEQ ID NO:71.

In some embodiments, the one or more bacteriophages may comprise a polynucleotide sequence with at least 91% identity to SEQ ID NO:73. In some embodiments, the one or more bacteriophages may comprise a polynucleotide sequence with at least 92% identity to SEQ ID NO:73. In some embodiments, the one or more bacteriophages may comprise a polynucleotide sequence with at least 93% identity to SEQ ID NO:73. In some embodiments, the one or more bacteriophages may comprise a polynucleotide sequence with at least 94% identity to SEQ ID NO:73. In some embodiments, the one or more bacteriophages may comprise a polynucleotide sequence with at least 95% identity to SEQ ID NO:73. In some embodiments, the one or more bacteriophages may comprise a polynucleotide sequence with at least 96% identity to SEQ ID NO:73. In some embodiments, the one or more bacteriophages may comprise a polynucleotide sequence with at least 97% identity to SEQ ID NO:73. In some embodiments, the one or more bacteriophages may comprise a polynucleotide sequence with at least 98% identity to SEQ ID NO:73. In some embodiments, the one or more bacteriophages may comprise a polynucleotide sequence with at least 99% identity to SEQ ID NO:73.

In some embodiments, the EPS depolymerase is alginate lyase, as described in detail elsewhere herein.

In some embodiments, one or more of the bacteriophages are engineered. In some embodiments, the one or more bacteriophages are genetically engineered. As used herein, “genetically engineered” or “genetically modified” bacteriophages may be bacteriophages whose polynucleotide sequence has been altered by genetic engineering techniques. Genetic engineering of polynucleotide sequences can be achieved by any modern molecular biology technique well known in the art, including but not limited to homologous recombination, bacteriophage engineering, CRISPR-based manipulation (e.g., CRISPR-Cas), reconstruction of full-length phage genomes in yeast or through in-vitro DNA splicing followed by transformation of full-length naked phage DNA into a host bacteria, and any combination of techniques thereof.

In some embodiments, two or more of the bacteriophages are engineered. In some embodiments, three or more of the bacteriophages are engineered. In some embodiments, four or more of the bacteriophages are engineered. In some embodiments, five or more of the bacteriophages are engineered. In some embodiments, six or more of the bacteriophages are engineered. In some embodiments, seven or more of the bacteriophages are engineered. In some embodiments, eight or more of the bacteriophages are engineered. In some embodiments, nine or more of the bacteriophages are engineered. In some embodiments, ten or more of the bacteriophages are engineered.

In some embodiments, a second bacteriophage of the one or more bacteriophages comprises a naturally occurring phage. The terms “naturally-occurring” and “wild-type” refer to a form found in nature. For example, a naturally occurring or wild-type nucleic acid molecule, nucleotide sequence or protein may be present in and isolated from a natural source, and is not intentionally modified by human manipulation. As such, the naturally occurring phage is a non-engineered phage that occurs in nature. In some embodiments, a second bacteriophage can comprise a mutated naturally occurring phage, and/or a partially or fully synthetic phage, particularly where the additional bacteriophage has the ability to infect, kill, or reduce a bacterial infection, as, for example, described in detail in WO 2016/100389, incorporated herein by reference, in its entirety.

In some embodiments, two or more of the bacteriophages are naturally occurring phages. In some embodiments, three or more of the bacteriophages are naturally occurring phages. In some embodiments, four or more of the bacteriophages are naturally occurring phages. In some embodiments, five or more of the bacteriophages are naturally occurring phages. In some embodiments, six or more of the bacteriophages are naturally occurring phages. In some embodiments, seven or more of the bacteriophages are naturally occurring phages. In some embodiments, eight or more of the bacteriophages are naturally occurring phages. In some embodiments, nine or more of the bacteriophages are naturally occurring phages. In some embodiments, ten or more of the bacteriophages are naturally occurring phages.

In some embodiments, one or more of the bacteriophages in a composition are engineered and one or more bacteriophages are naturally occurring. In some embodiments, one or more of the bacteriophages in a composition are engineered and two or more bacteriophages are naturally occurring. In some embodiments, one or more of the bacteriophages in a composition are engineered and three or more bacteriophages are naturally occurring. In some embodiments, one or more of the bacteriophages in a composition are engineered and four or more bacteriophages are naturally occurring. In some embodiments, one or more of the bacteriophages in a composition are engineered and five or more bacteriophages are naturally occurring. In some embodiments, two or more of the bacteriophages in a composition are engineered and one or more bacteriophages are naturally occurring. In some embodiments, three or more of the bacteriophages in a composition are engineered and one or more bacteriophages are naturally occurring. In some embodiments, four or more of the bacteriophages in a composition are engineered and one or more bacteriophages are naturally occurring. In some embodiments, five or more of the bacteriophages in a composition are engineered and one or more bacteriophages are naturally occurring. In some embodiments, any other combination of engineered and naturally occurring phages is envisioned.

In some embodiments, the bacteriophage targets Pseudomonas. In some embodiments, at least one of the bacteriophages target Pseudomonas aeruginosa. In some embodiments, the bacteriophage targets one or more Pseudomonas aeruginosa strains.

In some embodiments, the one or more bacteriophages of the composition targets one or more of Pseudomonas aeruginosa, antibiotic-resistant Pseudomonas aeruginosa, and multiple antibiotic-resistant Pseudomonas aeruginosa. In some embodiments, the one or more bacteriophages of the composition infect and kill one or more of Pseudomonas aeruginosa, antibiotic-resistant Pseudomonas aeruginosa, and multiple antibiotic-resistant Pseudomonas aeruginosa, as described above.

In some embodiments, the composition further comprises a storage medium for storage at room temperature or a temperature at or below 8° C. In embodiments, the bacteriophage composition includes a storage media for storage at a temperature at or below 7° C. In embodiments, the bacteriophage composition includes a storage media for storage at a temperature at or below 6° C. In embodiments, the bacteriophage composition includes a storage media for storage at a temperature at or below 5° C. In embodiments, the bacteriophage composition includes a storage media for storage at a temperature at or below 4° C. In embodiments, the bacteriophage composition includes a storage media for storage at a temperature at or below 3° C. In embodiments, the bacteriophage composition includes a storage media for storage at a temperature at or below 2° C. In embodiments, the bacteriophage composition includes a storage media for storage at a temperature at or below 1° C. In embodiments, the bacteriophage composition includes a storage media for storage at a temperature at or below 0° C. In embodiments, the bacteriophage composition includes a storage media for storage at a temperature at or below −20° C. In embodiments, the bacteriophage composition includes a storage media for storage at a temperature at or below −80° C.

In specific embodiments, the composition is stored at a temperature ranging from −20° C. to 25° C., such as at 20° C., 21° C., 22° C., 23° C., 24° C., or 25° C., or any value or subrange in between, including endpoints.

In specific embodiments, the composition is stored at a temperature ranging from 2° C. to 8° C., such as at 2° C., 3° C., 4° C., 5° C., 6° C., 7° C., or 8° C., or any value or subrange in between, including endpoints.

In specific embodiments, the composition is stored at room temperature.

In some embodiments, the storage medium comprises a cryoprotectant. In some embodiments, the cryoprotectant is glycerol, such as between about 5% and about 50% glycerol; more preferably between about 10% and about 30% glycerol; most preferably about 20% glycerol. In other embodiments, the cryoprotectant is sucrose, such as between about 5% to about 30% sucrose, most preferably about 10% sucrose. In some embodiments, the cryoprotectant is dimethylsulfoxide (DMSO), such as between about 2% to about 10% DMSO. Suitable concentrations may be any value or subvalue within the recited ranges, including endpoints.

In some embodiments, the bacteriophage composition may be used directly, refrigerated, cryodesiccated, lyophilized, stored frozen in aqueous or other solution with the appropriate cryoprotectant, as described above.

In some embodiments, the composition further comprises a pharmaceutically acceptable carrier, diluent, excipient or combinations thereof, as described in detail elsewhere herein.

In some embodiments, the composition is a liquid, semi-liquid, solid, frozen, or lyophilized formulation. In embodiments, the bacteriophage composition is in a liquid formulation. In embodiments, the bacteriophage composition is in a semi-liquid formulation. In embodiments, the bacteriophage composition is in a solid formulation. In embodiments, the bacteriophage composition is in a frozen formulation. In embodiments, the bacteriophage composition is in a lyophilized formulation.

In some embodiments, the composition comprises between 1×10⁸ and 1×10¹² PFU per milliliter of each bacteriophage. In some embodiments, the bacteriophage concentration is 1×10⁸ to 1×10⁹ PFU, 1×10⁸ to 1×10¹⁰ PFU, 1×10⁸ to 1×10¹¹ PFU, or 1×10⁸ to 1×10¹² PFU of each phage per ml of composition. In some embodiments, the bacteriophage concentration is 3×10⁸ to 1×10⁹ PFU, 3×10⁸ to 1×10¹⁰ PFU, 3×10⁸ to 1×10¹¹ PFU, or 3×10⁸ to 1×10¹² PFU of each phage per ml of composition. In some embodiments, the bacteriophage concentration is 3×10⁸ to 3×10⁹ PFU, or 3×10⁸ to 3×10¹⁰ PFU, or 3×10⁸ to 3×10¹² PFU of each phage per ml of composition. In some embodiments, the bacteriophage concentration is 1×10⁹ to 1×10¹⁰ PFU, or 1×10⁹ to 1×10¹ PFU, or 1×10⁹ to 1×10¹² PFU of each phage per ml of composition. In some embodiments, the bacteriophage concentration is 1×10¹⁰ to 1×10¹² PFU of each phage per ml of composition. In some embodiments, the bacteriophage concentration is 1×10¹² to 1×10¹² PFU of each phage per ml of composition. In some embodiments, the bacteriophage is administered to a subject at a dosage of at least about 1×10⁸ PFU of each phage, at least about 3×10⁸ PFU of each phage, at least about 1×10⁹ PFU of each phage, at least about 1×10¹⁰ PFU of each phage, at least about 1×10¹¹ PFU, or at least about 1×10¹² PFU of each phage per ml of composition. In embodiments, one or more bacteriophage(s) may be combined to form a total concentration of about 1×10⁸, about 3×10⁸, about 1×10⁹, about 1×10¹⁰, 1×10¹¹, or 1×10¹² PFU of each phage per ml of composition. Concentrations include any value, subvalue, range, or subrange within the recited ranges, including endpoints.

In some embodiments, the one or more bacteriophages of the composition reduce biofilm mass, as described in more detail in the examples.

### Methods for Treating Infections

In some embodiments, the present disclosure relates to a method for treating a Pseudomonas aeruginosa infection, comprising administering any of the compositions described herein to a subject in need thereof. The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a caregiver's expertise, but that include the knowledge that the subject is ill, or will be ill, as the result of a condition that is treatable by the compositions of the invention.

In some embodiments, provided herein are methods for treatment that include administration of a therapeutically effective amount of bacteriophage or a therapeutically effective amount of a bacteriophagre composition. In some embodiments, provided herein are methods for treatment that include administering a bacteriophage to a subject, where the bacteriophage includes a bacteriophage concentration range between about 1×10⁸ and about 1×10¹² PFU per ml of each bacteriophage. In some embodiments, the bacteriophage concentration is 1×10⁸ to 1×10⁹ PFU, 1×10⁸ to 1×10¹⁰ PFU, 1×10⁸ to 1×10¹¹ PFU, or 1×10¹² PFU of each phage per ml of composition. In some embodiments, the bacteriophage concentration is 3×10⁸ to 1×10⁹ PFU, 3×10⁸ to 1×10¹⁰ PFU, 3×10⁸ to 1×10¹¹ PFU, or 3×10⁸ to 1×10¹ PFU of each phage per ml of composition. In some embodiments, the bacteriophage concentration is 3×10⁸ to 3×10⁹ PFU, or 3×10⁸ to 3×10¹⁰ PFU of each phage per ml of composition. In some embodiments, the bacteriophage concentration is 1×10⁹ to 1×10¹⁰ PFU, 1×10⁹ to 1×10¹¹ PFU, 1×10⁹ to 1×10¹² PFU of each phage per ml of composition. In some embodiments, the bacteriophage concentration is 1×10¹⁰ to 1×10¹² PFU of each phage per ml of composition. In embodiments, one or more bacteriophage(s) may be combined and administered. In some embodiments, the one or more combined bacteriophage for administration can include two different engineered phage, such as any set forth herein. The two or more engineered phage can be engineered, for example, to each express a different EPS depolymerase or the same EPS depolymerase. The engineered phage can be from the same genus, for example as set forth herein, or from different genera. In some embodiments, it should be understood that one or more phage engineered to express a gene as set forth herein can be utilized in a medical treatment along with one or more phage that have not been so engineered (e.g., a phage that is not engineered at all or that does not include a depolymerase gene, for example). In some embodiments one or more bacteriophage(s) may be combined to form a total concentration of between about 1×10⁸ and about 5×10¹² PFU of each phage per ml of composition. In embodiments, one or more bacteriophage(s) may be combined to form a total concentration of between about 1×10⁸ and about 3×10¹² PFU of each phage per ml of composition. In embodiments, one or more bacteriophage(s) may be combined to form a total concentration of about 1×10⁸, 3×10⁸, 1×10⁹, 1×10¹⁰, 1×10¹¹, or 1×10¹² PFU of each phage per ml of composition. In embodiments, one or more bacteriophage(s) may be combined to form a total concentration of about 9×10⁸, 3×10⁹, 3×10¹⁰, 3×10¹¹, or 3×10¹² PFU of each phage per ml of composition. Concentrations include any value or range within the recited ranges, including endpoints.

In some embodiments, the bacteriophage is administered to a subject at a dosage of at least about 1×10⁸ PFU of each phage, at least about 3×10⁸ PFU of each phage, at least about 1×10⁹ PFU of each phage, at least about 1×10¹⁰ PFU of each phage, or at least about 1×10¹¹ PFU of each phage per dose. In some embodiments, the composition is administered at a dosage of at least 3×10⁸ PFU of total bacteriophages per dose. In some embodiments, the composition is administered at a dosage of at least 3×10⁹ PFU of total bacteriophages per dose. In some embodiments, the composition is administered at a dosage of at about 1×10⁸ PFU to about 5×10¹² PFU of total bacteriophages per dose. Doses include any value or range within the recited ranges, including endpoints.

In some embodiments, the method further comprises administration of an antibiotic. In embodiments, the antibiotic is selected from fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, quinolone, oxazolidinone, phenicol phosphonic acid, streptogramin, tetracycline, sulfonamide, imipenem, meropenem, amikacin, ciprofloxacin, levofloxacin, tobramycin, azithromycin, aztreonam, colistin, inhaled tobramycin, inhaled aztreonam, and inhaled colistin.

In some embodiments, the method further comprises administration of one or more CFTR modulators. In embodiments, the CFTR modulator may be selected from, but is not necessarily limited to, ivacaftor, lumacaftor/ivacaftor, tezacaftor/ivacaftor, elexacaftor/tezacaftor/ivacaftor, or any combination thereof.

In some embodiments, the bacterial infection has become resistant to one or more antibiotics or other treatments. In some embodiments, the bacterial infection has become resistant to one or more of a fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, quinolone, oxazolidinone, phenicol phosphonic acid, streptogramin, tetracycline, sulfonamide, imipenem, meropenem, amikacin, ciprofloxacin, levofloxacin, tobramycin, azithromycin, aztreonam, colistin, inhaled tobramycin, inhaled aztreonam, and inhaled colistin.

In some embodiments, the method can include treating, and optionally selecting for treatment, a subject who has not previously been treated with one or more antibiotics, or a subject that was previously treated with one or more antibiotics. For example, the phage treatment as described according to any of the embodiments set forth herein can be the first treatment (alone or combined with another type of treatment such as antibiotic treatment) given for a new infection or it can be a treatment that is given subsequent some other first treatment approach, such as for example, an antibiotic treatment. As noted, the methods can include selecting a subject or patient based upon that subject having already received a first treatment. That first treatment may not have been effective or may have only been partially effective. The infection may be one that has resistance or developed a resistance to another treatment, for example.

In embodiments, provided herein are methods for treating bacterial infection by administering any bacteriophage composition described herein in combination with fluoroquinolone. In embodiments, provided herein are methods for treating bacterial infection by administering any bacteriophage composition described herein in combination with carbapenem. In embodiments, provided herein are methods for treating bacterial infection by administering any bacteriophage composition described herein in combination with aminoglycoside. In embodiments, provided herein are methods for treating bacterial infection by administering any bacteriophage composition described herein in combination with ansamycin. In embodiments, provided herein are methods for treating bacterial infection by administering any bacteriophage composition described herein in combination with cephalosporin. In embodiments, provided herein are methods for treating bacterial infection by administering any bacteriophage composition described herein in combination with penicillin. In embodiments, provided herein are methods for treating bacterial infection by administering any bacteriophage composition described herein in combination with beta lactam. In embodiments, provided herein are methods for treating bacterial infection by administering any bacteriophage composition described herein in combination with beta lactamase inhibitor. In embodiments, provided herein are methods for treating bacterial infection by administering any bacteriophage composition described herein in combination with folate pathway inhibitor. In embodiments, provided herein are methods for treating bacterial infection by administering any bacteriophage composition described herein in combination with fucidane. In embodiments, provided herein are methods for treating bacterial infection by administering any bacteriophage composition described herein in combination with glycopeptide. In embodiments, provided herein are methods for treating bacterial infection by administering any bacteriophage composition described herein in combination with glycylcycline. In embodiments, provided herein are methods for treating bacterial infection by administering any bacteriophage composition described herein in combination with lincosamide. In embodiments, provided herein are methods for treating bacterial infection by administering any bacteriophage composition described herein in combination with lipopeptide. In embodiments, provided herein are methods for treating bacterial infection by administering any bacteriophage composition described herein in combination with macrolide. In embodiments, provided herein are methods for treating bacterial infection by administering any bacteriophage composition described herein in combination with quinolone. In embodiments, provided herein are methods for treating bacterial infection by administering any bacteriophage composition described herein in combination with oxazolidinone. In embodiments, provided herein are methods for treating bacterial infection by administering any bacteriophage composition described herein in combination with phenicol phosphonic acid. In embodiments, provided herein are methods for treating bacterial infection by administering any bacteriophage composition described herein in combination with streptogramin. In embodiments, provided herein are methods for treating bacterial infection by administering any bacteriophage composition described herein in combination with tetracycline. In embodiments, provided herein are methods for treating bacterial infection by administering any bacteriophage composition described herein in combination with sulfonamide. In embodiments, provided herein are methods for treating bacterial infection by administering any bacteriophage composition described herein in combination with imipenem. In embodiments, provided herein are methods for treating bacterial infection by administering any bacteriophage composition described herein in combination with meropenem. In embodiments, provided herein are methods for treating bacterial infection by administering any bacteriophage composition described herein in combination with amikacin. In embodiments, provided herein are methods for treating bacterial infection by administering any bacteriophage composition described herein in combination with ciprofloxacin. In embodiments, provided herein are methods for treating bacterial infection by administering any bacteriophage composition described herein in combination with levofloxacin. In embodiments, provided herein are methods for treating bacterial infection by administering any bacteriophage composition described herein in combination with tobramycin. In embodiments, provided herein are methods for treating bacterial infection by administering any bacteriophage composition described herein in combination with azithromycin. In embodiments, provided herein are methods for treating bacterial infection by administering any bacteriophage composition described herein in combination with aztreonam. In embodiments, provided herein are methods for treating bacterial infection by administering any bacteriophage composition described herein in combination with colistin. In embodiments, provided herein are methods for treating bacterial infection by administering any bacteriophage composition described herein in combination with inhaled tobramycin. In embodiments, provided herein are methods for treating bacterial infection by administering any bacteriophage composition described herein in combination with inhaled aztreonam. In embodiments, provided herein are methods for treating bacterial infection by administering any bacteriophage composition described herein in combination with inhaled colistin. Any one or more treatments described herein may be expressly excluded.

In some embodiments, the bacteriophage composition is administered via inhalation. In some embodiments, the bacteriophage composition is administered via nebulization.

In embodiments, provided herein are methods of administering to a subject any of the bacteriophage or composition described herein, where administration is over a range of about 3 to about 24 hours. In embodiments, the bacteriophage is administered to a subject every 3 hours. In embodiments, the bacteriophage is administered to a subject every 4 hours. In embodiments, the bacteriophage is administered to a subject every 5 hours. In embodiments, the bacteriophage is administered to a subject every 6 hours. In embodiments, the bacteriophage is administered to a subject every 7 hours. In embodiments, the bacteriophage is administered to a subject every 8 hours. In embodiments, the bacteriophage is administered to a subject every 9 hours. In embodiments, the bacteriophage is administered to a subject every 10 hours. In embodiments, the bacteriophage is administered to a subject every 11 hours. In some embodiments, the bacteriophage is administered to a subject every 12 hours. In embodiments, the bacteriophage is administered to a subject every 18 hours. In embodiments, the bacteriophage is administered to a subject every 24 hours. In some embodiments, the bacteriophage is administered to a subject at least once a day. Timing includes any value or range within the recited ranges, including endpoints.

In specific embodiments, the bacteriophage composition is administered at least every six hours.

In some embodiments, the bacteriophage composition is administered for at least one day. In embodiments, the bacteriophage composition is administered for a total of 2 days. In embodiments, the bacteriophage composition is administered for a total of 3 days. In embodiments, the bacteriophage composition is administered for a total of 4 days. In embodiments, the bacteriophage composition is administered for a total of 5 days. In embodiments, the bacteriophage composition is administered for a total of 6 days. In embodiments, the bacteriophage composition is administered for a total of 7 days. In embodiments, the bacteriophage composition is administered for a total of 10 days. In embodiments, the bacteriophage composition is administered for a total of 14 days. In embodiments, the bacteriophage composition is administered for a total of 21 days. In embodiments, the bacteriophage composition is administered for a total of 28 days. In embodiments, the bacteriophage composition is administered for between one day and about four weeks. The duration of administration may be any value or subrange within the recited ranges, including endpoints.

In some embodiments, the bacteriophages and/or compositions described herein may be administered in combination with one or more purified alginate lyases.

In some embodiments, the subject is human.

In some embodiments, the subject suffers from cystic fibrosis (CF). In some embodiments, the subject suffers from non-cystic fibrosis bronchiectasis (NFCB).

In other embodiments, the present disclosure also provides methods for treating a bacterial infection comprising: selecting a subject having a bacterial infection, and administering to the subject an effective amount of any of the bacteriophages described herein.

Subjects may be selected based on any of the following criteria. In some embodiments, the subject may have a bacterial infection that is a Pseudomonas infection. In some embodiments, the bacterial infection is a Pseudomonas aeruginosa infection. In some embodiments, the infection may include, or is associated with, but is not necessarily limited to, cystic fibrosis and/or pneumonia. In some embodiments, the infection comprises a bacterial infection in the presence of cystic fibrosis and/or pneumonia. In some embodiments, the infection may include, or is associated with, but is not necessarily limited to, non-cystic fibrosis bronchiectasis (NCFB). In some embodiments, the infection comprises a bacterial infection in the presence of NCFB and/or pneumonia. In some embodiments, the infection comprises mucoidy, or overproduction of alginate. In some embodiments, the bacterial infection is characterized by a biofilm. In some embodiments, the subject may have a Pseudomonas infection that is antibiotic resistant. In some embodiments, the subject may have a Pseudomonas infection that is resistant to multiple antibiotics. In some embodiments, the subject may be selected based upon having previously been treated for a bacterial infection with one or more antibiotics or another antibacterial treatment. In some embodiments the subject may be selected based upon having an infection by a bacterium that has developed resistance, or is resistant, to the initial antibiotic treatment or to one or more antibiotics.

### Methods for Making Engineered Bacteriophages

In some embodiments, the present disclosure provides a method for making an engineered bacteriophage, comprising providing a bacteriophage and incorporating an exopolysaccharide (EPS) depolymerase into the bacteriophage.

In some embodiments, the EPS depolymerase may be, but is not necessarily limited to, alginate lyase. In some embodiments, the alginate lyase may include, but is not necessarily limited to, Alg2A or A1-III, as described in detail elsewhere herein. In some embodiments, the alginate lyase sequences described herein may include portions of, or functional fragments of an alginate lyase gene. In some embodiments, the sequences may include a whole alginate lyase gene.

In some embodiments, the bacteriophage belongs to the Genus Phikmvvirus. In some embodiments, the bacteriophage belongs to the Genus Pakpunavirus. In some embodiments, the bacteriophage belongs to the Genus Bruynoghevirus. In some embodiments, the bacteriophage belongs to the Genus Pbunavirus. In some embodiments, the bacteriophage belongs to the Genus Luzseptimavirus. In some embodiments, the bacteriophage belongs to the Genus Litunavirus. In some embodiments, the bacteriophage belongs to the Genus Nankokuvirus

In some embodiments, the bacteriophages and compositions described herein encode regulatory elements, which may include, but are not necessarily limited to, promoters, cis-elements, enhancers, terminators, or introns. In some aspects of the invention, gene expression from the nucleic acid and/or phage encoding the inserted gene or EPS depolymerase is regulated by a promoter to which the nucleic acid is operatively linked. The term “promoter”, “promoter region” or “promoter sequence” refers to a nucleic acid sequence capable of binding RNA polymerase to initiate transcription of a gene in the 51 to 3 (“downstream”) direction. A gene is “under the control of” or “regulated by” a promoter when RNA polymerase binding to the promoter is a proximal cause of transcription of the gene. The promoter or promoter region typically provides a recognition site for RNA polymerase and other factors necessary for proper transcription initiation. The promoter may be isolated from the 5′ untranslated region (5′ UTR) of the genomic copy of the gene. Alternatively, promoters can be produced or designed synthetically by altering known DNA elements. Chimeric promoters that combine the sequence of one promoter with the sequence of another promoter are also contemplated. Promoters may be defined by their expression pattern based on, for example, metabolic, environmental, or developmental conditions. A promoter can be used as a regulatory element for regulating the expression of an operably linked transcribable polynucleotide molecule (e.g., a coding sequence). In addition to sequences recognized by RNA polymerase (and preferably other transcription factors), promoters may also contain regulatory elements, such as cis-elements or enhancer domains, that affect transcription of operably linked genes. A “viral promoter” is a native or non-native promoter that initiates transcription of one or more genes located within the viral genome.

The promoter(s) can be constitutive promoter(s) and/or conditional promoter(s) and/or inducible promoter(s) and/or tissue specific promoter(s). As used herein, the term “constitutive” promoter refers to a promoter that is active tinder most environmental and developmental conditions. Constitutive promoters are active regardless of the external environment such as light and medium composition. In some examples, a constitutive promoter is active in the presence and absence of a nutrient. For example, a constitutive promoter may be a promoter that is active (mediates transcription of a gene to which it is operably linked) under nitrogen-depleted conditions as well as under conditions in which nitrogen is not limiting (nitrogen-replete conditions). Conversely, an “inducible” promoter is a promoter that responds to a particular environmental condition (such as the presence or absence of nutrients or regulators, the presence of light, etc.).

The promoter can be selected from the group consisting of RNA polymerases, pol I, pol II, pol III, T7, M13, promoters recognized by bacterial sigma factors (σ⁷⁰, σ⁵⁴, σ^{S}, σ³², σ¹⁹, σ²⁸ or σ³⁸), for example, Pᵣᵣₙ, P_{lac}, P_{tac}, Pₜₑₜ, P_{bla}, P_{cat}, P_{bad}, P_{L} or any natural or synthetic, constitutive or inducible promoter known to those skilled in the art.

In some embodiments, the promoter is a promoter that is naturally occurring in the bacteriophage. Thus, a promoter operably linked to a gene to which it is not operably linked in its native state (e.g., in the genome of a non-genetically engineered organism or virus) is referred to herein as a “heterologous promoter,” even though the promoter may be derived from the same species (or in some cases, the same organism or virus) as the gene to which it is linked. Similarly, when referring to a protein localization sequence or protein domain of an engineered protein, “heterologous” means that the localization sequence or protein domain is derived from a protein that is different from the protein into which it is integrated by genetic engineering.

As used herein, the term “operably linked” refers to a configuration in which a control sequence is placed at an appropriate position relative to the coding sequence of a polynucleotide sequence such that the control sequence directs or regulates the expression of the coding sequence of a polypeptide and/or functional RNA. Thus, a promoter is operably linked to a nucleic acid sequence if it can mediate transcription of the nucleic acid sequence. When introduced into a host cell, the expression cassette can result in transcription and/or translation of the encoded RNA or polypeptide under suitable conditions. Antisense or sense constructs that are not translated or cannot be translated are not excluded by this definition. In the case of expression of a transgene and suppression of an endogenous gene, the ordinarily skilled artisan will recognize that the inserted polynucleotide sequence need not be identical, but may be only substantially identical to the sequence of the gene from which it is derived. As explained herein, these substantially identical variants are specifically encompassed by reference to a particular nucleic acid sequence.

In some aspects of the invention, gene expression from the nucleic acid and/or phage encoding the inserted gene or EPS depolymerase is regulated by a terminator to which the nucleic acid is operatively linked to. As used herein, the term “terminator” or “terminator sequence” or “transcription terminator” refers to the regulatory region of a genetic sequence that causes RNA polymerase to stop transcription. In some embodiments, the terminator is a terminator that is naturally occurring in the bacteriophage. In some embodiments, the terminator is a terminator that is not naturally occurring in the bacteriophage.

In some embodiments, the engineered viruses described herein may include codon-optimized sequences. As used herein, the term “codon-optimized” means a polynucleotide, nucleic acid sequence, or coding sequence has been redesigned as compared to a wild-type or reference polynucleotide, nucleic acid sequence, or coding sequence by choosing different codons without altering the amino acid sequence of the encoded protein. Accordingly, codon-optimization generally refers to replacement of codons with synonymous codons to optimize expression of a protein while keeping the amino acid sequence of the translated protein the same. Codon optimization of a sequence can increase protein expression levels (Gustafsson et al., Codon bias and heterologous protein expression. 2004, Trends Biotechnol 22: 346-53) of the encoded proteins, for example, and provide other advantages. Variables such as codon usage preference as measured by codon adaptation index (CAI), for example, the presence or frequency of U and other nucleotides, mRNA secondary structures, cis-regulatory sequences, GC content, and other variables may correlate with protein expression levels (Villalobos et al., Gene Designer: a synthetic biology tool for constructing artificial DNA segments. 2006, BMC Bioinformatics 7:285).

Any method of codon optimization can be used to codon optimize polynucleotides and nucleic acid molecules provided herein, and any variable can be altered by codon optimization. Accordingly, any combination of codon optimization methods can be used. Exemplary methods include the high codon adaptation index (CAI) method, the Low U method, and others. The CAI method chooses a most frequently used synonymous codon for an entire protein coding sequence. As an example, the most frequently used codon for each amino acid can be deduced from 74,218 protein-coding genes from a human genome. The Low U method targets U-containing codons that can be replaced with a synonymous codon with fewer U moieties, generally without changing other codons. If there is more than one choice for replacement, the more frequently used codon can be selected. Any polynucleotide, nucleic acid sequence, or codon sequence provided herein can be codon-optimized.

In some embodiments, any of the EPS depolymerases or alginate lyases described herein may be codon optimized for any of the sequences described herein, as needed. Any method known in the art for optimizing codons may be used.

The engineered phage described herein may be made using any method for genetically engineering viruses, including bacteriophage, that are known in the art. Non-limiting examples are provided in U.S. Pat. Nos. 5,811,093, 8,865,158; and 10,837,004, each of which is incorporated herein by reference in its entirety for all methods, compositions, reagents, and all other information provided therein.

In some embodiments, the present disclosure also provides an assay for determining alginate lyase activity of an engineered bacteriophage, comprising administering an effective amount of any of the engineered bacteriophages described herein to a Pseudomonas aeruginosa biofilm and determining reduction in biofilm mass.

### Kits

In some embodiments, the present disclosure provides one or more kits comprising any of the bacteriophages or bacteriophage compositions described herein, and instructions for using the same. In some embodiments, the kits may include one or more purified alginate lyases for co-administration with the bacteriophage composition.

In some embodiments, the kit may further comprise an antibiotic. Suitable antibiotics include, but are not necessarily limited to, fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, quinolone, oxazolidinone, phenicol phosphonic acid, streptogramin, tetracycline, sulfonamide, imipenem, meropenem, amikacin, ciprofloxacin, levofloxacin, tobramycin, azithromycin, aztreonam, colistin, inhaled tobramycin, inhaled aztreonam, and inhaled colistin, as described elsewhere herein.

In some embodiments, the kit further comprises one or more CFTR modulators. In embodiments, the CFTR modulator may be selected from, but is not necessarily limited to, ivacaftor, lumacaftor/ivacaftor, tezacaftor/ivacaftor, elexacaftor/tezacaftor/ivacaftor, or any combination thereof.

In some embodiments, the kit may further comprise a means of administering the bacteriophage or bacteriophage composition. The means may include, but is not necessarily limited to, one or more syringes, transdermal patches, slow-release devices, sprays, nebulizers, an inhalers, or respirators.

In some embodiments, the slow-release device may comprise a mini-osmotic pump.

In some embodiments, the kit may further comprise a second bacteriophage or bacteriophage composition.

### EXAMPLES

### Example 1: Activity of Alginate Lyase in Various Bacteriophages Alginate Lyase A1-III Fragment 70-399 Expresses Poorly and has No Activity but Fragment 54-412 does

The alginate lyase gene of Sphingomonas, aly, produces a pre-pro-protein. The full size protein is proteolytically cleaved to generate AlgL A1-II and A1-III. The latter carries the bacterial alginate degradation activity of Aly.

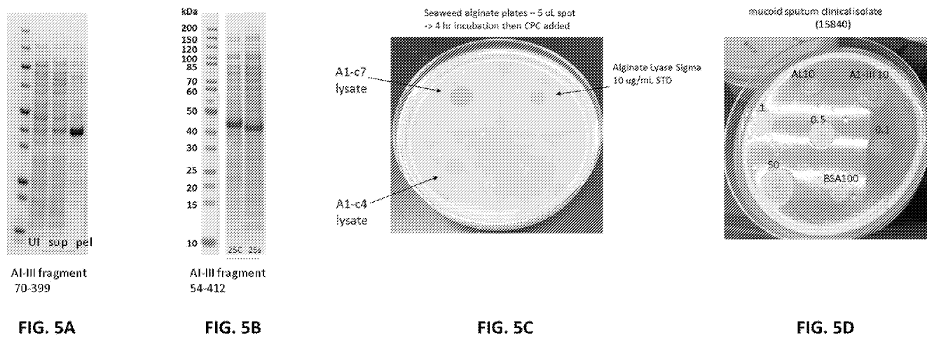

Fragments 70-399 or 54-412 of the Sphingomonas aly (alginate lyase) gene were cloned into an E. coli expression plasmid with a C-terminal his-tag and purified using Ni affinity columns. Fragment 70-399 was mostly insoluble and required denaturation of the aggregate and refolding for purification (FIG. 5A). Fragment 54-412 of the Sphingomonas aly (alginate lyase) gene was cloned into an E. coli expression plasmid with a C-terminal his-tag and purified using Ni affinity columns. Fragment 54-412 was soluble (FIG. 5B). The resulting purified proteins were over 99% pure upon examination of denaturing gels and tested for their ability to degrade alginate from seaweed or bacteria. Activity was compared to that of a commercially available alginate lyase preparation from Sigma-Aldrich (ref A1603) prepared at 10 mg/ml. Activity is assessed on agar plates containing 2% seaweed alginate. 5 μl samples of preparation or control were applied to the surface of the plate, allowed to dry, and then incubated for 4 hrs-overnight at 30° C. The plates were then stained with 10% cetylpyridinium chloride (CPC) and activity was revealed as halos where no white precipitate was visible (FIG. 5C). Activity was also assessed on preformed lawns of mucoid, alginate expressing strains of Pseudomonas aeruginosa where activity presents as a halo of rougher more transparent lawn at the site of application. Presented in FIG. 5D are the results on a lawn of strain 15840.

Phages Expressing AlgL A1-III Fragment 70-399 have No Alginate Lyase Activity

Phage APBP4-4 is a mutant of APBP4 carrying the gene for the AlgL A1-III fragment 70-399 inserted downstream of gp99 but shows no alginate lyase activity. APBP17-1 is a mutant of APBP17 carrying the gene for the AlgL A1-III fragment 70-399 and also shows no alginate lyase activity.

### Phages APBP1-1, APBP1-2, APBP3-1, and APBP3-2 Display Robust Alginate Lyase Activity

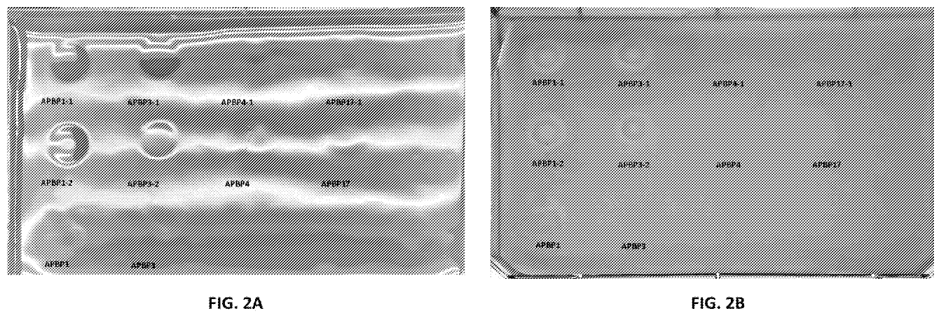

APBP1-1 and APBP1-2 are mutants of Pbunavirus APBP1 with fragment 54-412 of Aly cloned immediately downstream of gene gp82. APBP1-1 has a version of algL A1-III that is codon optimized for E. coli while the version of the gene cloned in APBP1-2 is codon optimized for Pseudomonas aeruginosa (Pae). Both have 6-His-tag at their C-terminus. APBP3-1 and APBP3-2 are mutants of Phikmvvirus APBP3. APBP3-1 carries the same algL A1-III allele as APBP1-2. APBP3-2 carries the same algL A1-III allele as APBP1-1. AlgL A1-III is inserted downstream of gp037. Each phage displays alginate lyase activity in the Preformed Pae strain 15844 lawn assay whereas the parental phages have none. FIG. 2A shows a top-lit view of phage spotted on preformed lawns of mucoid, alginate expressing strains of Pseudomonas aeruginosa (strain 15844) where activity presents as a crater or indentation lawn at the site of application. FIG. 2B shows a bottom-lit view of the same plate seen in 13A, where phage spotted on preformed lawns of mucoid, alginate expressing strains of Pseudomonas aeruginosa (strain 15844) can be seen as circular indentations on the lawn at the site of application.

### Western Blotting Detection of AlgL A1-III-His6 in Lysates of APBP1-1, APBP1-2, APBP3-1 or APBP3-2

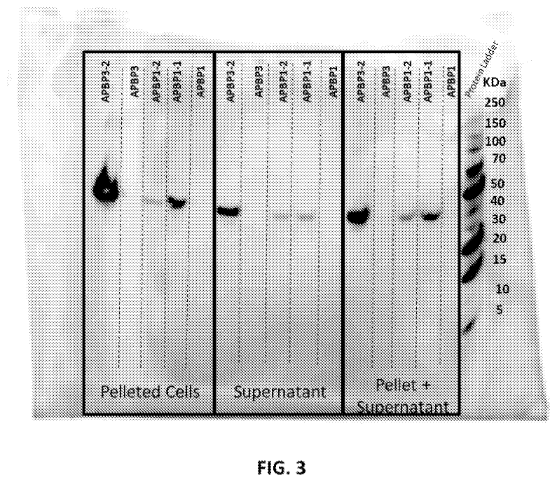

Whole lysates of each phage were prepared for denaturing SDS-PAGE, electrophoresed, transferred to a PVDF membrane and His-tagged proteins revealed using a primary antibody against His-tag and an appropriate HRP-conjugated secondary antibody. Alginate lyase (AlgL) A1-III fragment 54-412 shows as a ˜37 kDa band (FIG. 3).

### Growth of Phages APBP1-1, APBP1-2, APBP3-1 and APBP3-2 is not Altered by Expression of AlgL A1-III

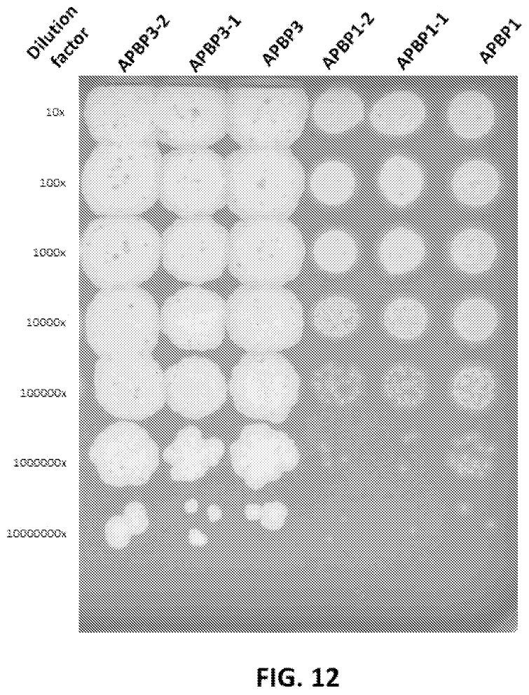

Lysates of APBP3-1 and APBP3-2 started from a single plaque picked out of a lawn of their host, Pseudomonas aeruginosa strain 7299, and resuspended in an exponentially growing culture of Pseudomonas aeruginosa strain 7299 reached the same titer as their parental phage APBP3. APBP1-1 and APBP1-2 grew to titers about ten-fold lower than that of the parental phage APBP1 (FIG. 12).

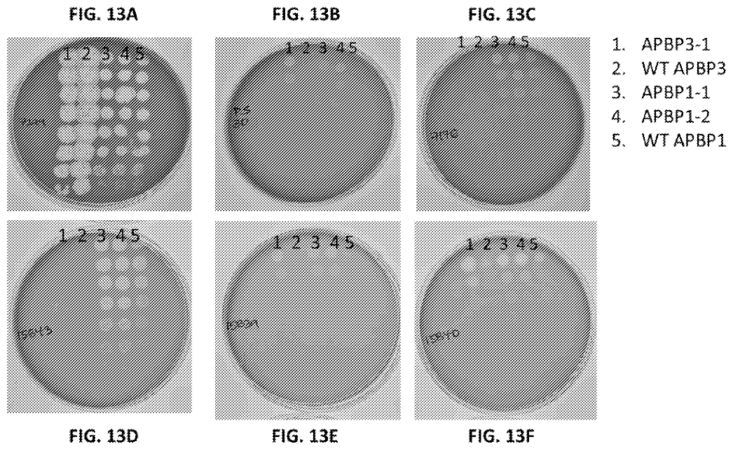

Host range of phages APBP1-1, APBP1-2, APBP3-1 and APBP3-2 was expanded by expression of AlgL A1-III (see FIGS. 13A-13F).

### Example 2: Methods of Generating Engineered Bacteriophages

One method of generating engineered bacteriophages expressing an alginate lyase from their genome involves the construction of a plasmid that carries the alginate lyase gene with appropriate expression signals (promoters and or ribosome binding site) cloned in between two fragments of target phage genomes that will direct the alginate lyase gene to the appropriate locus through homologous recombination with the corresponding loci in the wild-type phage. These so-called “homology arms” may be as short as 50 bp and as long as 2 kb but are typically ˜500 bp. In addition, the plasmid carries a single guide RNA (sgRNA) under constitutive expression. sgRNA are synthetic guide RNAs that work in concert with the programmable Cas9 nuclease to effect double-stranded cuts a few base pairs downstream of the sequence to which the sgRNA is complementary. An sgRNA/Cas9 complex therefore represents a sequence-directed nuclease that can be used as a counter-selection system for particular sequences. Here, the sgRNA/Cas9 was used to counter-select the wild-type target phage and favor the growth of recombinants that have acquired the alginate lyase gene. This was achieved by choosing a gRNA sequence that is destroyed or deleted during the recombination process such that the sgRNA/Cas9 complex is not capable of targeting the genome of recombinants. The design of sgRNAs is well-known to people skilled in the art. Cas9 is provided in specially constructed strains of Pseudomonas aeruginosa, which are sensitive to the target phage and express Cas9 from an IPTG-inducible promoter. The plasmid carrying the homology arm-surrounded alginate lyase and sgRNA was transformed into the appropriate Cas9-expressing strain. The resulting transformant was grown in the presence of IPTG and infected with the wild-type phage. The resulting lysate was then plated on a lawn of the host for the target phage and individual plaques analyzed for the presence of the alginate lyase gene in the target locus. Apparent efficiency of recombination was in the 1-100% range, most typically ˜10% ( 1/10 phage plaque was a correct recombinant). Analysis for the proper insertion of the alginate lyase gene can be functional, PCR-based and/or can be done by full genome sequencing using, for example, Illumina NGS.

Another method for generating engineered bacteriophages relies on work by Ando et al (US2015/0064770 A1). Briefly, the entire genome of the target phage is amplified by PCR in pieces overlapping by 20-500 bp (most ideally 50 bp). At each end of the phage genomes, the oligonucleotides used for amplification carry homology to a yeast artificial chromosome (YAC). Co-transformation of all the overlapping phage genomic fragments and an appropriate YAC in the yeast Saccharomyces cerevisiae leads to the assembly of a yeast-replicative circular plasmid that can be selected using the selection marker present on the YAC fragment (most commonly Leucine auxotrophy) with the entire phage genome reassembled inside of it. Because yeast and bacteria have completely different gene expression machineries, the phage genome is kept inactive. The reconstructed phage-YAC can then be extracted from the yeast cells, transformed into a highly competent bacterial strain (most commonly E. coli DH10B or its derivatives), where it will be re-activated to produce progeny that can then be plated on the appropriate host; in this case Pseudomonas aeruginosa, grown and analyzed for correctness. As PCR oligonucleotides can be chosen anywhere on the genome of phage to generate the overlapping genomic fragments, it is possible to make insertions, deletions or mutations anywhere in the phage and to make multiple modifications in a single re-assembly experiment (for example delete a sequence while also replacing it with another sequence or delete a sequence in one locus while inserting another sequence elsewhere in the genome). Adequate primer design allows the insertion of the alginate lyase gene at any desired locus of the target phage genome as long as the requirements are met: 1) maintenance of at least a 20 bp overlap between each consecutive PCR fragment; and 2) no inactivation of any essential phage function in the reconstructed genome.

### Example 3: Specific Engineered Bacteriophages

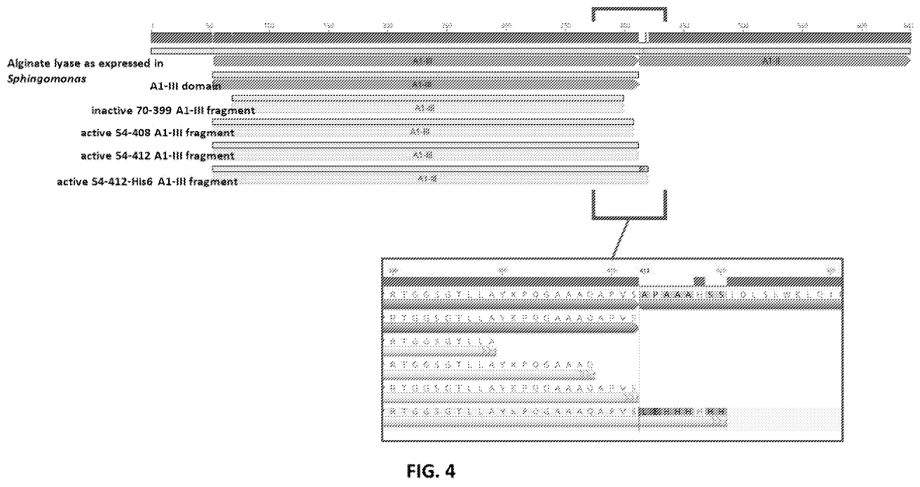

A1-III is an N-terminal fragment of Sphingomonas sp. Alginate lyase Aly (accession number BAB03312), and corresponds to amino acids 54-412 of Aly. In an attempt to save coding space in engineered phages, cloning of fragment 70-399 (FIG. 4) was attempted but it showed no alginate activity. However, both fragments 54-412 and 54-408 produced alginate lyase activity. In some constructs, a C-terminal His₆ tag was appended for detection or purification using anti-His-tag antibodies. The inset in FIG. 4 shows the differences between several sequences around the C-terminus of the various A1-III fragments studied at the amino acid level.

The alginate lyase gene of Sphingomonas, aly, produces a pre-pro-protein. The full-size protein is proteolytically cleaved to generate AlgL A1-II and A1-III. The latter carries the bacterial alginate degradation activity of Aly. Fragments 70-399 or 54-412 of the Sphingomonas aly (alginate lyase) gene were cloned into an E. coli expression plasmid with a C-terminal His₆-tag and purified using Ni affinity columns. Fragment 70-399 was mostly insoluble and required denaturation of the aggregate and refolding for purification (FIG. 5A). A1-III fragment 54-412 shows a band at the correct size on the Western blot, indicating it is expressed (FIG. 5B). The resulting purified proteins were over 99% pure upon examination of denaturing gels and tested for their ability to degrade alginate from seaweed or bacteria. Activity was compared to that of a commercially available alginate lyase preparation from Sigma-Aldrich (ref A1603) prepared at 10 mg/ml. Activity was assessed on agar plates containing 2% seaweed alginate. 5 μl samples of preparation or control were applied to the surface of the plate, allowed to dry, and then incubated for 4 hrs to overnight at 30° C. The plates were finally stained with calcium phosphate cement (CPC) and activity was revealed as halos where no white precipitate is visible (FIG. 5C). Activity was also assessed on preformed lawns of mucoid, alginate-expressing strains of Pseudomonas aeruginosa where activity presents as a halo of rougher, more transparent lawn at the site of application. The results are presented on a mucoid lawn of alginate-producing Pseudomonas aeruginosa strain 15844 (FIG. 5D).

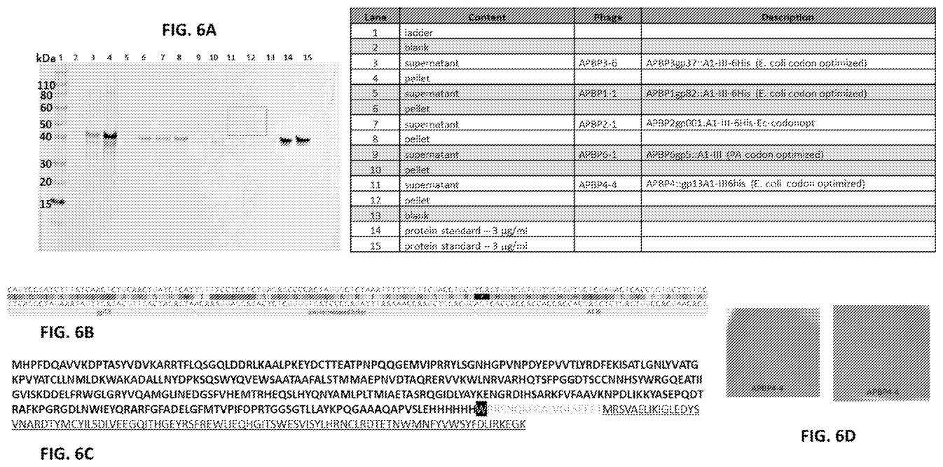

The alginate lyase A1-III was expressed as a fusion protein with gp13 when engineered downstream of APBP4 gp13.1. FIG. 6A shows a Western blot of lysates of engineered phages carrying the A1-III alginate lyase payload. The engineered phages were used to infect an exponentially growing culture of Pseudomonas aeruginosa clinical isolate DCF47 at an MOI of 1. The phage and host bacterial cells were grown together, shaking at 37° C., then harvested at 250 minutes post-infection. The lysate was centrifuged to concentrate the cellular debris. The cell pellet and the supernatant were separated and frozen at −80° C. Each sample was subsequently prepared for denaturing SDS-PAGE, electrophoresed, transferred to a PVDF membrane and A1-III proteins were revealed using a primary antibody against A1-III and an appropriate Alkaline Phosphatase secondary antibody. The alginate lyase A1-III fragment 54-412 shows as a ˜37 kDa band. The band for APBP4-4 (APBP4 (gp13.1 A1-III gp13)) in lanes 11 and 12 appears at ˜55 kDa instead of ˜43 kDa, most likely due to a read-through of the gene in which the A1-III payload was inserted. This indicates that a fusion protein is produced, rather than the correctly sized A1-III protein alone. FIG. 6B shows the DNA sequence around the C-terminus of A1-III, illustrating how trans-reading the TGA stop codon of A1-III-His₆ could lead to an in-frame fusion protein with gp13 through a 19 aa-long “anti-terminated linker”. FIG. 6C shows the putative sequence of the A1-III-His₆-linker-gp13 fusion protein generated in lysates of APBP4-4. Residues in bold correspond to the A1-III-His₆ sequence, underlined resides correspond to gp13, grey residues denote the putative linker and the black highlighted tryptophan residue is a likely way for the cell to misread a TGA stop codon of A1-III-His₆. APBP4-4 shows no activity on a mucoid lawn of alginate-producing Pseudomonas aeruginosa strain 15844, indicating that the fusion protein is non-functional (FIG. 6D).

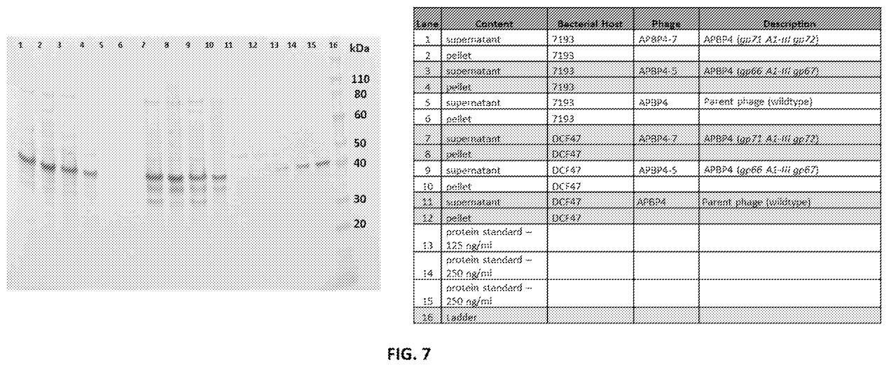

Engineered phages carrying the A1-III alginate lyase payload were generated. The engineered phages were used to infect an exponentially growing culture of Pseudomonas aeruginosa strain 7193 or clinical isolate DCF47, at an MOI of 1. The phage and host bacterial cells were grown together, shaking at 37° C., then harvested at 250 minutes post-infection. The lysates were centrifuged to concentrate the cellular debris. The cell pellet and the supernatant were separated and frozen at −80° C. Each sample was subsequently prepared for denaturing SDS-PAGE, electrophoresed, transferred to a PVDF membrane, and A1-III proteins revealed using a primary antibody against A1-III and an appropriate Alkaline Phosphatase secondary antibody. These versions of engineered APBP4 show the correctly sized band (FIG. 7), indicating A1-III expression and absence of fusion to surrounding proteins.

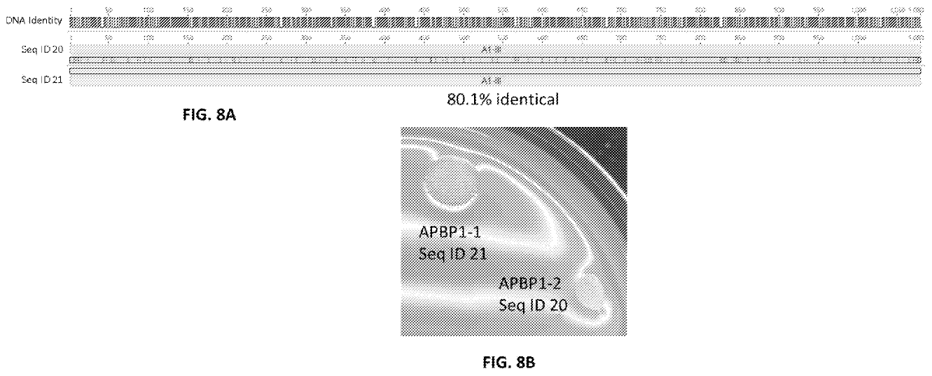

Two codon usage matrices for alginate lyase A1-III were cloned into phages. Both were cloned in the same locus in APBP1 to generate APBP1-1 and APBP1-2. SEQ ID NO:34 has a higher GC content than SEQ ID NO:35, and is presumably more optimized for expression in P. aeruginosa, whereas SEQ ID NO:35 is more optimized for expression in E. coli k-12. Overall, the two genes encode identical proteins, but are only 80.1% identical at the DNA level (FIG. 8A). Both codon optimizations of A1-III engineered into phage APBP1 showed alginate lyase activity on a mucoid lawn of P. aeruginosa 15844 (FIG. 8B).

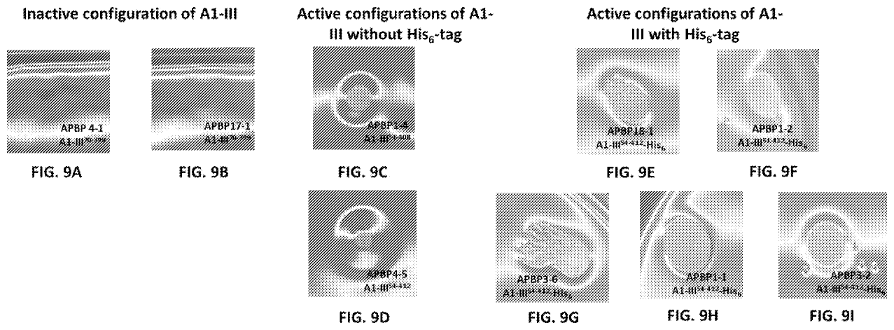

Examples of alginate lyase activity profiles from lysates of engineered phages expressing various configurations of A1-III are shown in FIGS. 9A-9I. Activity was assessed on preformed lawns of mucoid, alginate-expressing strains of Pseudomonas aeruginosa where activity presents as a halo of rougher, more transparent lawn at the site of application. The results shown in FIGS. 9A-9I were observed on a lawn of strain 15844. Fragment 70-399, when engineered into the genomes of phages APBP4 (FIG. 9A) or APBP17 (FIG. 9B) shows no activity. The 54-408 and 54-412 fragments of A1-III show activity in any of 5 different phages: the 54-408 fragment of SEQ ID NO:35 cloned into APBP1 (FIG. 9C); the 54-412 fragment from SEQ ID NO:35 cloned into APBP4 (FIG. 9D); the 54-412 fragment from SEQ ID NO:35 with a C-terminal His₆-tag, cloned into APBP18 (FIG. 9E) and APBP1 (FIG. 9F); the 54-412 fragment from SEQ ID NO:34 with a C-terminal His₆-tag, cloned into APBP3 (FIGS. 9G and 9I) and APBP1 (FIG. 9H).

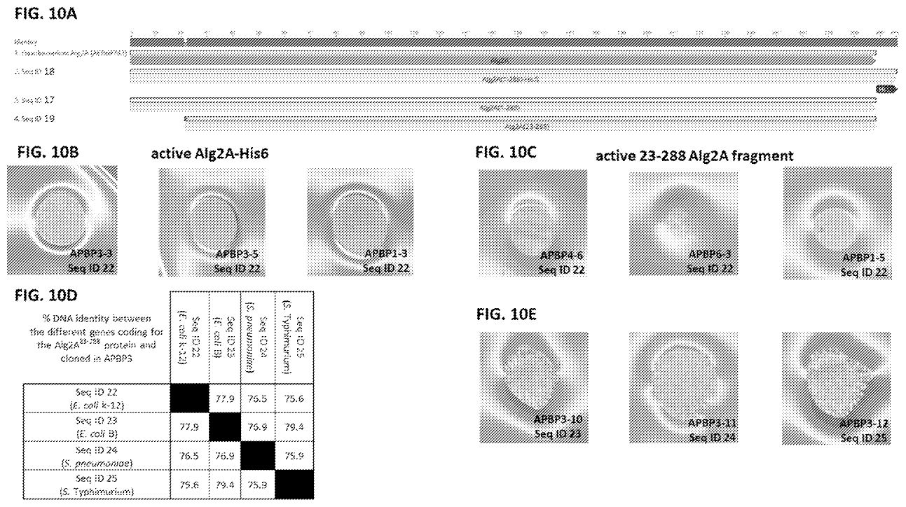

A subsequent set of experiments showed that Flavobacterium Alg2A (accession number AEB69783) expressed from multiple phages has alginate lyase activity whether the gene retains its N-terminal signal sequence, is tagged with a C-terminal His₆-tag, or is encoded by 4 different genes. Phages expressing the full-length Alg2A with a C-terminus His tag all produce lysates displaying alginate lyase activity on mucoid lawns of alginate-producing Pseudomonas aeruginosa strain 15844 (FIG. 10B). Phages expressing the truncated signal sequence-deleted version of Alg2A²³⁻²⁸⁸ without a His₆-tag all produce lysates displaying alginate lyase activity on mucoid lawns of alginate-producing Pseudomonas aeruginosa strain 15844 (FIG. 10C). Four different codon usage matrices were used to synthesize alg2A genes and clone them into phage APBP3. SEQ ID NO:36 is presumably optimized for expression in E. coli k-12, SEQ ID NO:37 for E. coli B, SEQ ID NO:38 for S. pneumoniae, and SEQ ID NO:39 for S. enterica serovar Typhimurium. The genes are 75-9-77.9% identical to one another (FIG. 10D). APBP3-derived phages expressing Alg2A²³⁻²⁸⁸ from SEQ ID NO:37, SEQ ID NO:38, or SEQ ID NO:39 produce lysates displaying alginate lyase activity on mucoid lawns of alginate-producing Pseudomonas aeruginosa strain 15844 (FIG. 10E).

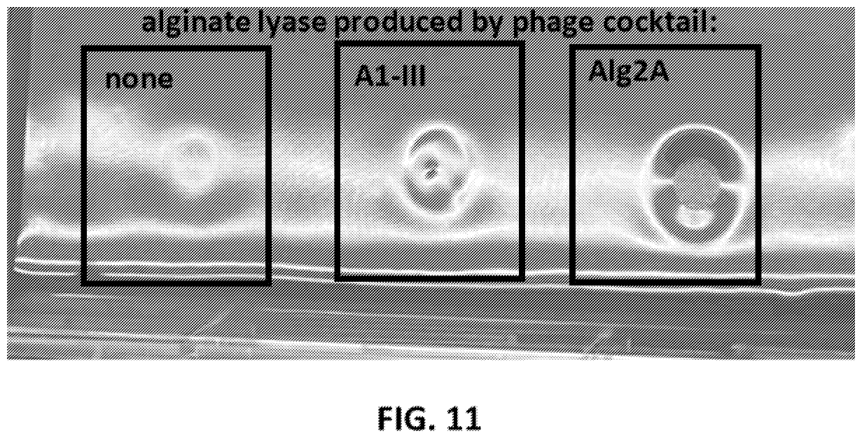

To determine whether a cocktail of phages engineered with either A1-III or Alg2A alginate lyase have enzymatic activity, a preformed mucoid lawn of Pseudomonas aeruginosa strain FRD1 was spotted with 10 microliters of phage mixtures, then incubated at 30° C. before being photographed. The left most spot in FIG. 11 is a mixture of wild-type parent phages: APBP4, APBP1, APBP2, APBP6, and APBP3. No clearing of the mucoid lawn is visible. The center spot is a mixture of the same phages, engineered to express A1-III: APBP2-1, APBP3-6, APBP6-1, APBP1-4, APBP4-5. A clearing is visible in the shiny surface of the mucoid lawn, suggesting that the enzyme digested the available alginate. The spot on the right side is a mixture of phages, engineered to express Alg2A: APBP2-2, APBP3-5, APBP6-3, APBP1-5, APBP4-6. A crater-like clearing is visible in the shiny surface of the mucoid lawn, suggestive of increased alginate lyase activity.

Each of the phages tested reached the same titer as their parental phages APBP1 and APBP3, which did not express alginate lyase, suggesting that growth of Phages APBP1-1, APBP1-2, APBP3-1 and APBP3-2 is not altered by expression of alginate lyase protein A1-III (FIG. 12). The titer was calculated from the plaque forming units (PFU) counts and the dilution factor for each lysate.

To determine the effect of expression of alginate lyase A1-III on host range of phages, the titer of lysates of parental phages and their engineered derivatives was measured, first, on their host, Pseudomonas aeruginosa 7299. Dilutions series are 10-fold steps for all phages and all plates. As seen in FIG. 13A, phages APBP3-1, wild-type APBP3, APBP1-1, APBP1-2, and wild-type APBP1 all produced the same titer. On Pseudomonas aeruginosa strain PS 30, APBP3-1 shows clearing down to the 10² dilution while its parent APBP3 does not produce plaques at all on this strain (FIG. 13B). Similarly, APBP1-1 and APBP1-2 produce plaques on Pseudomonas aeruginosa strain PS 30 while their parent APBP1 does not (FIG. 13B). APBP1-1 and APBP1-2 show improved clearing on Pseudomonas aeruginosa strain 7176 compared to their parent APBP1 (FIG. 13C). APBP1-1 and APBP1-2 plaque down to 10⁻⁷ dilution (˜2E8 PFU/ml) on Pseudomonas aeruginosa strain 15843 while their parent APBP1 plaques around ten-fold less to 10⁻⁶ dilution (˜2E7 PFU/ml) (FIG. 13D). On Pseudomonas aeruginosa strain 15839, APBP3-1 shows clearing while its parent APBP3 does not produce plaques at all on this strain (FIG. 13E). APBP1-1 and APBP1-2 also produce clearings on Pseudomonas aeruginosa strain 15839 while their parent APBP1 does not (FIG. 13E). APBP3-1 shows increased clearing compared to parent APBP3 on Pseudomonas aeruginosa strain 15840 (FIG. 13F). APBP1-1 and APBP1-2 also show improved clearing on Pseudomonas aeruginosa strain 15840 compared to APBP1 (FIG. 13F). These results suggest that the host range of phages APBP1-1, APBP1-2, APBP3-1 and APBP3-2 is expanded by expression of alginate lyase A1-III.

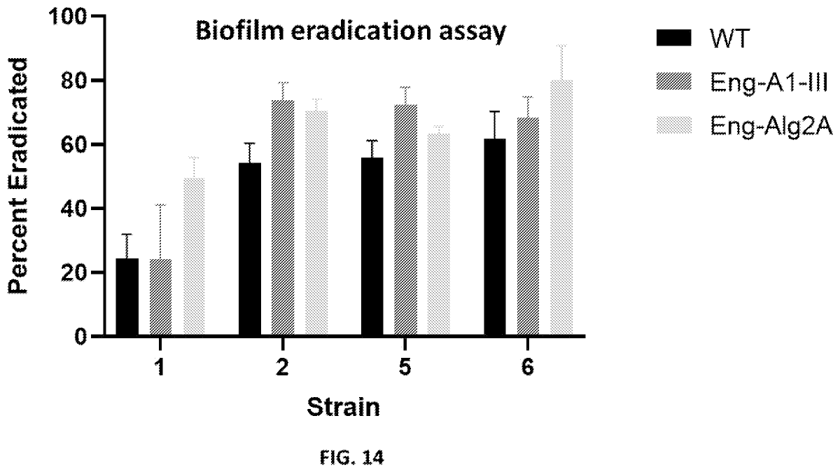

Four different strains of Pseudomonas aeruginosa were grown as biofilms on pegs using an MBEC (minimum biofilm eradication concentration) assay kit (Innovotech MBEC assay kit or any other similar assays known in the art may be used). Three versions of a 5-phage cocktail (AP-PA02) were each applied to the biofilms. After treatment, biofilms were washed and stained with crystal violet. The stain was extracted, then analyzed for density at OD595. The two cocktails of engineered phages, containing phage expressing either A1-III (Eng-A1-III) or Alg2A (Eng-Alg2A), showed increased disruption of the biofilm, up to a 30% increase in strain 1, when compared to the wild-type phage cocktail (WT; FIG. 14). These results suggest that biofilms treated with engineered phages are disrupted more than biofilms treated with the wild-type phage counterparts.

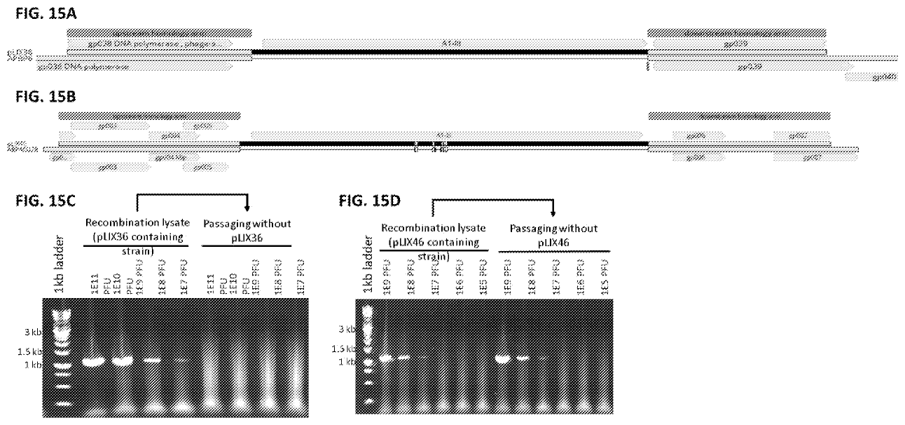

The recombination between expression plasmid pLIX36 carrying A1-III and APBP6 to integrate A1-III between gp038 and gp039 is represented in FIG. 15A: a strain carrying a plasmid, pLIX36, harboring the 520 bp upstream of base pair 20585 of APBP6 (upstream homology arm; end of gp038), the A1-III gene and the 500 bp downstream of base pair 20586 (downstream homology arm; beginning of gp039) was infected with APBP6, allowing recombination between the plasmid and phage and generating an engineered APBP6 derivative with A1-III replacing bp T20585. The recombination between pLIX46 and APBP6 to integrate A1-III between gp005 and gp006 is represented in FIG. 15B: a strain carrying a plasmid, pLIX46, harboring the 437 bp upstream of base pair 2501 of APBP6 (upstream homology arm; gp003-005), the A1-III gene and the 501 bp downstream of base pair 2540 (downstream homology arm; beginning of gp039) was infected with APBP6, allowing recombination between the plasmid and phage and generating an engineered APBP6 derivative with A1-III replacing bp 2502-2539.

A PCR assay was performed to verify the presence of inserted A1-III in APBP6 lysates grown on a pLIX36-containing strain and then passaged on a plasmid-less host. A ˜1.1 kb band indicates presence of engineered phage (FIG. 15C). The APBP6 lysate grown in the presence of pLIX36 was serially diluted and assayed by PCR for the presence of engineered phages. The recombination lysate was then passaged on the host of APBP6 without pLIX36 and assayed by PCR again (FIG. 15C). Recombinants were no longer detected, indicating the engineered phages were not viable when cultured without pLIX36. A PCR assay was also performed to verify the presence of inserted A1-III in APBP6 lysates grown on a pLIX46 containing strain, and then passaged on a plasmid-less host. A ˜1.1 kb band indicates presence of engineered phage (FIG. 15D). The APBP6 lysate grown in the presence of pLIX46 was serially diluted and assayed by PCR for the presence of engineered phages. The recombination lysate was then passaged on the host of APBP6 without pLIX46 and assayed by PCR again. Recombinants were still detectable, indicating that the engineered phage were viable when cultured without pLIX36 (FIG. 15D). These results suggested that although engineering of A1-III in APBP6 is possible at both the gp038 gp039 and gp005 gp006 loci, viable expression of the enzyme only occurs from the gp005 gp006 locus.

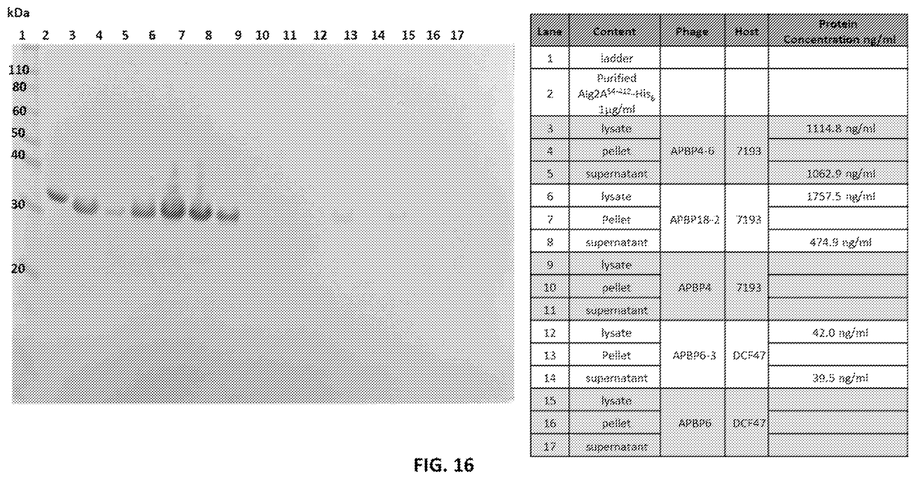

FIG. 16 shows a Western blot of lysates of engineered phages carrying the alg2A²³⁻²⁸⁸ alginate lyase gene. The engineered phages were used to infect an exponentially growing culture of their bacterial hosts: Pseudomonas aeruginosa clinical isolates 7193 or DCF47 at an MOI of 1. The phage and host bacterial cells were grown together, shaking at 37° C., then harvested at 250 minutes post-infection. The lysate was comprised of lysed cells and phage in the media. To separate the lysate into a pellet and a supernatant, it was centrifuged to concentrate the cellular debris. The cell pellet and the supernatant were separated and frozen at −80° C. Each sample was subsequently prepared for denaturing SDS-PAGE, electrophoresed, transferred to a PVDF membrane, and Alg2A proteins were revealed using a primary antibody against A1-III and an appropriate Alkaline Phosphatase secondary antibody. The alginate lyase Alg2A fragment shows as a ˜32 kDa band. ELISA protein quantification data are shown in the right-most column of the chart in FIG. 16, indicating that protein levels are pharmaceutically relevant. These results suggest that engineered phages ABP4-6, ABP18-2, ABP6-3 express alginate lyase protein Alg2A²³⁻²⁸⁸.

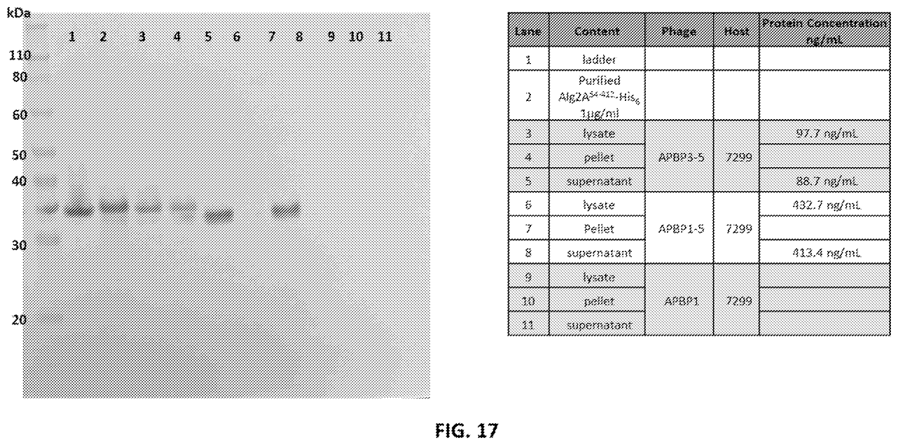

FIG. 17 shows a Western blot of lysates of engineered phages carrying different versions of the alg2A²³²⁸⁸ alginate lyase gene. The engineered phages were used to infect an exponentially growing culture of their bacterial hosts: Pseudomonas aeruginosa clinical isolates 7193 or DCF47 at an MOI of 1. The phage and host bacterial cells were grown together, shaking at 37° C., then harvested at 250 minutes post-infection. The lysate was comprised of lysed cells and phage in the media. To separate the lysate into a pellet and a supernatant, it was centrifuged to concentrate the cellular debris. The cell pellet and the supernatant were separated and frozen at −80° C. Each sample was subsequently prepared for denaturing SDS-PAGE, electrophoresed, transferred to a PVDF membrane, and Alg2A proteins were revealed using a primary antibody against A1-III and an appropriate Alkaline Phosphatase secondary antibody. The full-length Alg2A protein shows as a ˜35 kDa band, whereas the Alg2A²³⁻²⁸⁸ fragment migrates as a ˜32 kDa band. ELISA protein quantification data are shown in the right-most column of the chart in FIG. 17, indicating that protein levels are pharmaceutically relevant. These results suggest that engineered phages APBP3-5 and APBP1-5 express alginate lyase protein Alg2A¹⁻²⁸⁸ and Alg2A²³⁻²⁸⁸, respectively.

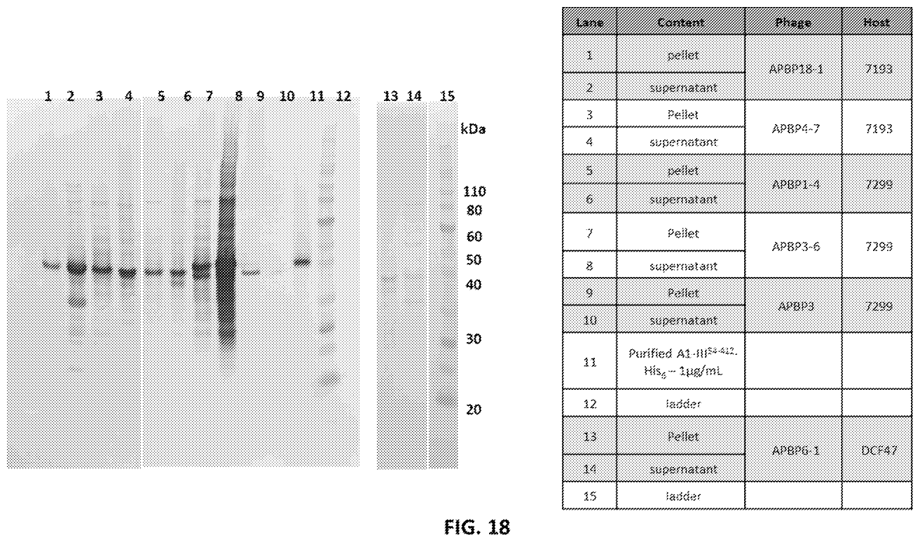

FIG. 18 shows a Western blot of lysates of engineered phages carrying the A1-III⁵⁴⁻⁴¹² or A1-III⁵⁴⁻⁴⁰⁸ alginate lyase gene. The engineered phages were used to infect an exponentially growing culture of their bacterial hosts: Pseudomonas aeruginosa clinical isolates 7193 or DCF47 at an MOI of 1. The phage and host bacterial cells were grown together, shaking at 37° C., then harvested at 250 minutes post-infection. The lysate was comprised of lysed cells and phage in the media. To separate the lysate into a pellet and a supernatant, it was centrifuged to concentrate the cellular debris. The cell pellet and the supernatant were separated and frozen at −80° C. Each sample was subsequently prepared for denaturing SDS-PAGE, electrophoresed, transferred to a PVDF membrane and Alg2A proteins revealed using a primary antibody against A1-III and an appropriate Alkaline Phosphatase secondary antibody. The alginate lyase A1-III fragment shows as a ˜40 kDa band. These results suggest that engineered phages APBP18-1, APBP4-7, and APBP6-1 express alginate lyase protein A1-III⁵⁴⁻⁴¹, APBP3-6 produces alginate lyase protein A1-III⁵⁴⁻⁴¹²-His₆, and that APBP1-4 expresses alginate lyase protein A1-III⁵⁴⁻⁴⁰⁸.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the claims.

What is claimed is:

1. A bacteriophage engineered to express an exopolysaccharide (EPS) depolymerase.

2. The bacteriophage of claim 1, wherein the EPS depolymerase is expressed from a nucleotide sequence selected from SEQ ID NO:20, SEQ ID NO:21, SEQ ID NO:22, SEQ ID NO:23, SEQ ID NO:24, SEQ ID NO:25, SEQ ID NO:36, or SEQ ID NO:59, or a sequence having at least 90% identity to the sequence of SEQ ID NO:20, at least 90% identity to the sequence of SEQ ID NO:21, at least 90% identity to the sequence of SEQ ID NO:22, at least 90% identity to the sequence of SEQ ID NO:23, at least 90% identity to the sequence of SEQ ID NO:24, at least 90% identity to the sequence of SEQ ID NO:25, at least 90% identity to the sequence of SEQ ID NO:36, or at least 90% identity to the sequence of SEQ ID NO:59.

3. The bacteriophage of claim 1 or 2, wherein the EPS depolymerase is alginate lyase.

4. The bacteriophage of claim 3, wherein the alginate lyase comprises Alg2A or A1-III.

5. The bacteriophage of claim 4, wherein the alginate lyase comprises Alg2A.

6. The bacteriophage of claim 4, wherein the alginate lyase comprises A1-III.

7. The bacteriophage of any of claims 1 to 6, wherein the bacteriophage shows improved host range.

8. The bacteriophage of any of claims 1 to 7, wherein the bacteriophage belongs to the Genus Phikmvvirus.

9. The bacteriophage of any of claims 1 to 7, wherein the bacteriophage belongs to the Genus Pakpunavirus.

10. The bacteriophage of any of claims 1 to 7, wherein the bacteriophage belongs to the Genus Bruynoghevirus.

11. The bacteriophage of any of claims 1 to 7, wherein the bacteriophage belongs to the Genus Pbunavirus.

12. The bacteriophage of any of claims 1 to 11, wherein the bacteriophage targets Pseudomonas aeruginosa.

13. The bacteriophage claim 12, wherein the bacteriophage targets one or more of Pseudomonas aeruginosa, antibiotic-resistant Pseudomonas aeruginosa, and multiple antibiotic-resistant Pseudomonas aeruginosa.

14. The bacteriophage of claim 13, wherein the bacteriophage infects and kills one or more of Pseudomonas aeruginosa, antibiotic-resistant Pseudomonas aeruginosa, and multiple antibiotic-resistant Pseudomonas aeruginosa.

15. The bacteriophage of any of claims 1 to 14, wherein the bacteriophage reduces biofilm mass.

16. A bacteriophage composition comprising one or more bacteriophages that express an exopolysaccharide (EPS) depolymerase, wherein the one or more bacteriophages comprise a polynucleotide sequence selected from SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:37; SEQ ID NO:38; SEQ ID NO:39; SEQ ID NO: 40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO: 52; SEQ ID NO: 53; SEQ ID NO:54; SEQ ID NO:55; SEQ ID NO:56; SEQ ID NO:57; SEQ ID NO:58; SEQ ID NO:60; SEQ ID NO:61; SEQ ID NO: 62; SEQ ID NO: 63; SEQ ID NO:64; SEQ ID NO:65; SEQ ID NO:66; SEQ ID NO:67; SEQ ID NO:68; SEQ ID NO:69; SEQ ID NO: 70; SEQ ID NO:71, SEQ ID NO: 73; a polynucleotide sequence with at least 90% identity to SEQ ID NO:26; a polynucleotide sequence with at least 90% identity to SEQ ID NO:27; a polynucleotide sequence with at least 90% identity to SEQ ID NO:28; a polynucleotide sequence with at least 90% identity to SEQ ID NO:29; a polynucleotide sequence with at least 90% identity to SEQ ID NO:30; a polynucleotide sequence with at least 90% identity to SEQ ID NO:31; a polynucleotide sequence with at least 90% identity to SEQ ID NO:32; a polynucleotide sequence with at least 90% identity to SEQ ID NO:33; a polynucleotide sequence with at least 90% identity to SEQ ID NO:34; a polynucleotide sequence with at least 90% identity to SEQ ID NO:35; a polynucleotide sequence with at least 90% identity to SEQ ID NO:37; a polynucleotide sequence with at least 90% identity to SEQ ID NO:38; a polynucleotide sequence with at least 90% identity to SEQ ID NO:39; a polynucleotide sequence with at least 90% identity to SEQ ID NO:40; a polynucleotide sequence with at least 90% identity to SEQ ID NO:41; a polynucleotide sequence with at least 90% identity to SEQ ID NO:42; a polynucleotide sequence with at least 90% identity to SEQ ID NO:43; a polynucleotide sequence with at least 90% identity to SEQ ID NO:44; a polynucleotide sequence with at least 90% identity to SEQ ID NO:45; a polynucleotide sequence with at least 90% identity to SEQ ID NO:46; a polynucleotide sequence with at least 90% identity to SEQ ID NO:47; a polynucleotide sequence with at least 90% identity to SEQ ID NO:48; a polynucleotide sequence with at least 90% identity to SEQ ID NO:49; a polynucleotide sequence with at least 90% identity to SEQ ID NO:50; a polynucleotide sequence with at least 90% identity to SEQ ID NO:51; a polynucleotide sequence with at least 90% identity to SEQ ID NO: 52; a polynucleotide sequence with at least 90% identity to SEQ ID NO:53; a polynucleotide sequence with at least 90% identity to SEQ ID NO: 54; a polynucleotide sequence with at least 90% identity to SEQ ID NO:55; a polynucleotide sequence with at least 90% identity to SEQ ID NO:56; a polynucleotide sequence with at least 90% identity to SEQ ID NO:57; a polynucleotide sequence with at least 90% identity to SEQ ID NO:58; a polynucleotide sequence with at least 90% identity to SEQ ID NO:60; a polynucleotide sequence with at least 90% identity to SEQ ID NO:61; a polynucleotide sequence with at least 90% identity to SEQ ID NO:62; a polynucleotide sequence with at least 90% identity to SEQ ID NO:63; a polynucleotide sequence with at least 90% identity to SEQ ID NO:64; a polynucleotide sequence with at least 90% identity to SEQ ID NO:65; a polynucleotide sequence with at least 90% identity to SEQ ID NO:66; a polynucleotide sequence with at least 90% identity to SEQ ID NO:67; a polynucleotide sequence with at least 90% identity to SEQ ID NO:68; a polynucleotide sequence with at least 90% identity to SEQ ID NO:69; a polynucleotide sequence with at least 90% identity to SEQ ID NO:70; a polynucleotide sequence with at least 90% identity to SEQ ID NO:71; a polynucleotide sequence with at least 90% identity to SEQ ID NO:73.

17. The bacteriophage composition of claim 16, wherein the EPS depolymerase is alginate lyase.

18. The bacteriophage composition of claim 16 or 17, wherein one or more of the bacteriophages are engineered.

19. The bacteriophage composition of any of claims 16 to 18, wherein two or more of the bacteriophages are engineered.

20. The bacteriophage composition of claim 16, wherein a second bacteriophage of the one or more bacteriophages comprises a naturally occurring phage.

21. The bacteriophage composition of claim 16, wherein two or more bacteriophages of the one or more bacteriophages are naturally occurring phages.

22. The bacteriophage composition of any of claims 16 to 21, wherein at least one of the bacteriophages target Pseudomonas aeruginosa.

23. The bacteriophage composition of claim 22, wherein the one or more bacteriophages of the composition targets one or more of Pseudomonas aeruginosa, antibiotic-resistant Pseudomonas aeruginosa, and multiple antibiotic-resistant Pseudomonas aeruginosa.

24. The bacteriophage composition of claim 23, wherein the one or more bacteriophages of the composition infect and kill one or more of Pseudomonas aeruginosa, antibiotic-resistant Pseudomonas aeruginosa, and multiple antibiotic-resistant Pseudomonas aeruginosa.

25. The bacteriophage composition of any of claims 16 to 24, further comprising a storage medium for storage at room temperature or a temperature at or below 8° C.

26. The bacteriophage composition of any of claims 16 to 24, wherein the composition is stored at a temperature ranging from −20° C. to 25° C.

27. The bacteriophage composition of claim 26, wherein the composition is stored at 2° C. to 8° C.

28. The bacteriophage composition of claim 26, wherein the composition is stored at room temperature.

29. The bacteriophage composition of claim 25, wherein the storage medium is for storage at 4° C., 0° C., −20° C., or −80° C.

30. The bacteriophage composition of claim 30, wherein the storage medium comprises a cryoprotectant.

31. The bacteriophage composition of claim 30, wherein the cryoprotectant comprises glycerol.

32. The bacteriophage composition of claim 31, wherein the composition comprises between about 5% and about 50% glycerol.

33. The bacteriophage composition of claim 32, wherein the storage medium comprises about 20% glycerol.

34. The bacteriophage composition of any of claims 16 to 33, wherein the composition further comprises a pharmaceutically acceptable carrier, diluent, excipient or combinations thereof.

35. The bacteriophage composition of claim 30, wherein the cryoprotectant comprises sucrose.

36. The bacteriophage composition of claim 35, wherein the composition comprises between about 5% and about 30% sucrose.

37. The bacteriophage composition of claim 36, wherein the composition comprises about 10% sucrose.

38. The bacteriophage composition of claim 30, wherein the cryoprotectant comprises dimethylsulfoxide (DMSO).

39. The bacteriophage composition of claim 38, wherein the DMSO is at a concentration of between 2% and 10%.

40. The bacteriophage composition of any of claims 16 to 39, wherein the composition is a liquid, semi-liquid, solid, frozen, or lyophilized formulation.

41. The bacteriophage composition of any of claims 16 to 40, wherein the composition comprises between 1×10⁸ and 1×10¹² PFU per milliliter of each bacteriophage.

42. The bacteriophage composition of any of claims 16 to 41, wherein the one or more bacteriophages of the composition reduce biofilm mass.

43. A method for treating a Pseudomonas aeruginosa infection, comprising administering the composition of any of claims 16 to 41 to a subject in need thereof.

44. The method of claim 43, wherein the composition is administered at a dosage of at least 3×10⁸ PFU of total bacteriophages per dose.

45. The method of claim 43 or 44, wherein the method further comprises administration of an antibiotic.

46. The method of claim 45, wherein the antibiotic is selected from the group consisting of fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, quinolone, oxazolidinone, phenicol phosphonic acid, streptogramin, tetracycline, sulfonamide, imipenem, meropenem, amikacin, ciprofloxacin, levofloxacin, tobramycin, azithromycin, aztreonam, colistin, inhaled tobramycin, inhaled aztreonam, and inhaled colistin.

47. The method of any of claims 43 to 46, wherein the method further comprises administration of one or more CFTR modulators selected from ivacaftor; lumacaftor and ivacaftor; tezacaftor and ivacaftor; elexacaftor, tezacaftor, and ivacaftor; or any other combination thereof.

48. The method of any of claims 43 to 47, wherein the bacterial infection has become resistant to one or more antibiotics selected from a fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, quinolone, oxazolidinone, phenicol phosphonic acid, streptogramin, tetracycline, sulfonamide, imipenem, meropenem, amikacin, ciprofloxacin, levofloxacin, tobramycin, azithromycin, aztreonam, colistin, inhaled tobramycin, inhaled aztreonam, and inhaled colistin.

49. The method of any of claims 43 to 48, wherein the bacteriophage composition is administered via inhalation.

50. The method of any of claims 43 to 48, wherein the bacteriophage composition is administered via nebulization.

51. The method of any of claims 43 to 48, wherein the bacteriophage composition is administered intravenously.

52. The method of any of claims 43 to 51, wherein the bacteriophage composition is administered at least once a day.

53. The method of any of claims 43 to 52, wherein the bacteriophage composition is administered for at least one day.

54. The method of any of claims 43 to 53, wherein the subject is human.

55. The method of any of claims 43 to 54, wherein the subject suffers from cystic fibrosis (CF).

56. The method of any of claims 43 to 54, wherein the subject suffers from non-cystic fibrosis bronchiectasis (NCFB).

57. The method of any of claims 43 to 56, wherein the subject was previously treated with one or more antibiotics.

58. An assay for determining alginate lyase activity of an engineered bacteriophage, comprising administering an effective amount of the engineered bacteriophage of any of claims 1 to 15 to a Pseudomonas aeruginosa biofilm and determining reduction in biofilm mass.

59. A method for treating a bacterial infection comprising:
(a) selecting a subject having a bacterial infection, and
(b) administering to the subject an effective amount of a bacteriophage of any one of claims 1-15, or a bacteriophage composition of any one of claims 16-42, thereby treating the bacterial infection.

60. The method of claim 59, wherein the bacterial infection is a Pseudomonas infection.

61. The method of claim 59, wherein the bacterial infection is a Pseudomonas aeruginosa infection.

62. The method of any one of claims 59-61, wherein the bacterial infection is characterized by a biofilm.

63. The method of any of claims 59 to 62, wherein the subject has cystic fibrosis (CF).

64. The method of any of claims 59 to 62, wherein the subject has non-cystic fibrosis bronchiectasis (NCFB).

65. The method of any one of claims 59 to 64, wherein the composition is administered at a dosage of at least 3×10⁸ PFU of total bacteriophages per dose.

66. The method of any one of claims 59 to 65, wherein the method further comprises administration of an antibiotic.

67. The method of claim 66, wherein the antibiotic is selected from the group consisting of fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, quinolone, oxazolidinone, phenicol phosphonic acid, streptogramin, tetracycline, sulfonamide, imipenem, meropenem, amikacin, ciprofloxacin, levofloxacin, tobramycin, azithromycin, aztreonam, colistin, inhaled tobramycin, inhaled aztreonam, and inhaled colistin.

68. The method of any of claims 59 to 67, wherein the method further comprises administration of one or more CFTR modulators selected from ivacaftor; lumacaftor and ivacaftor; tezacaftor and ivacaftor; elexacaftor, tezacaftor, and ivacaftor; or any other combination thereof.

69. The method of any one of claims 59 to 68, wherein the bacterial infection has become resistant to one or more antibiotics selected from a fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, quinolone, oxazolidinone, phenicol phosphonic acid, streptogramin, tetracycline, sulfonamide, imipenem, meropenem, amikacin, ciprofloxacin, levofloxacin, tobramycin, azithromycin, aztreonam, colistin, inhaled tobramycin, inhaled aztreonam, and inhaled colistin.

70. The method of any one of claims 59 to 69, wherein the bacteriophage composition is administered via inhalation.

71. The method of any one of claims 59 to 69, wherein the bacteriophage composition is administered via nebulization.

72. The method of any one of claims 59 to 71, wherein the bacteriophage composition is administered at least once a day.

73. The method of any one of claims 59 to 72, wherein the bacteriophage composition is administered for at least one day.

74. The method of any one of claims 59 to 73, wherein the subject is human.

75. A method for making an engineered bacteriophage, comprising providing a bacteriophage and incorporating an exopolysaccharide (EPS) depolymerase into the bacteriophage.

76. The method of claim 78, wherein the EPS depolymerase is alginate lyase.

77. The method of claim 75 or 76, wherein the alginate lyase comprises Alg2A or A1-III.

78. The method of claim 77, wherein the alginate lyase comprises Alg2A.

79. The method of claim 77, wherein the alginate lyase comprises A1-III.

80. The method of any of claims 75 to 79, wherein the bacteriophage belongs to the Genus Phikmvvirus.

81. The method of any of claims 75 to 79, wherein the bacteriophage belongs to the Genus Pakpunavirus.

82. The method of any of claims 75 to 79, wherein the bacteriophage belongs to the Genus Bruynoghevirus.

83. The method of any of claims 75 to 79, wherein the bacteriophage belongs to the Genus Pbunavirus.

84. A kit comprising a bacteriophage of anyone of claims 1-15, or a bacteriophage composition of any one of claims 16-42, and instructions for using the same.

85. The kit of claim 84, further comprising an antibiotic.

86. The kit of claim 84 or 85, wherein the kit further comprises one or more CFTR modulators selected from ivacaftor; lumacaftor and ivacaftor; tezacaftor and ivacaftor; elexacaftor, tezacaftor, and ivacaftor; or any other combination thereof.

87. The kit of any of claims 84 to 86, further comprising a means of administering the bacteriophage or bacteriophage composition.

88. The kit of claim 87, wherein the means comprises a syringe, a transdermal patch, a slow-release device, a spray, a nebulizer, an inhaler, or a respirator.

89. The kit of claim 88, wherein the slow-release device comprises a mini-osmotic pump.

90. The kit of any one of claims 84 to 89, further comprising a second bacteriophage or bacteriophage composition.

91. A bacteriophage composition comprising one or more bacteriophages engineered to express an exopolysaccharide (EPS) depolymerase, wherein the one or more bacteriophages belong to the Genus Phikmvvirus, Pakpunavirus, Bruynoghevirus, and/or Pbunavirus.
